(19) United States
(12) Patent Application Publication
(10) Pub. No.: US 2004/0101531 A1
(43) Pub. Date: May 27, 2004

(54) Immunogenic compositions and vaccines comprising carrier bacteria that secrete antigens

(76) Inventors: Roy Curtiss III, St. Louis, MO (US); Ho Young Kang, Pusan (KR)

Correspondence Address: THOMPSON COBURN, LLP, ONE US BANK PLAZA, SUITE 3500, ST LOUIS, MO, 63101, US

(21) Appl. No.: 10/414,532

(22) Filed: Apr. 15, 2003

Related U.S. Application Data
(60) Provisional application No. 60/372,710, filed on Apr. 16, 2002.

(51) Int. Cl. A61K 39/00; A61K 39/38

(52) U.S. Cl. 424/184.1

(57) ABSTRACT

Disclosed are vaccines and immunogenic compositions which use live attenuated pathogenic bacteria, such as Salmonella, to deliver ectopic antigens to the mucosal immune system of vertebrates. The attenuated pathogenic bacteria are engineered to secrete the antigen into the periplasmic space of the bacteria or into the environment surrounding the bacteria. The vertebrate mounts a Th2-mediated immune response toward the secreted antigen.

### CROSS-REFERENCE TO RELATED APPLICATIONS

This application claims priority to provisional application No. 60/372,710 filed Apr. 15, 2002.

### GOVERNMENTAL SUPPORT

This work was supported by the National Institutes of Health grant number DE 06669. The government of the United States of America has certain rights in this invention.

### SEQUENCE LISTING

A paper copy of the sequence listing and a computer readable form of the same sequence listing are appended below and herein incorporated by reference. The information recorded in computer readable form is identical to the written sequence listing, according to 37 C.F.R. 1.821 (f).

### BACKGROUND OF THE INVENTION

1. Field of the Invention

The invention relates generally to vaccines or immunogenic compositions comprising live attenuated bacteria used as carriers to deliver antigens to a vertebrate, wherein an immune response, especially a Th2-mediated immune response, is elicited by the vertebrate toward the antigen.

2. Description of the Related Art

Citations to some of the related art documents may be indicated as numbers in parentheses. Those numbered citations refer to the bibliography that is found at the end of this section. Those related art documents, as well as other references cited throughout this application, are herein incorporated by reference. The inclusion of those related art documents in this application is not an admission that those documents constitute prior art.

Approximately one-third of the 55 to 60 million human deaths each year are the result of infectious diseases caused by a diversity of pathogens including bacteria, fungi, protozoa, parasites (such as helminths) and viruses. Most of these pathogens are unicellular, although some fungi and parasites are multicellular. Viral pathogens are unable to multiply outside of living hosts and are composed of nucleic acids, polypeptides and sometimes lipids and carbohydrates. Infections by pathogens also cause significant morbidity associated with malnutrition and retarded development especially in children, and suboptimal work performance or absences from work of afflicted adults. The social and economic consequences of infectious diseases are staggering and difficult to compute. It is becoming increasingly clear that many disease states such as ulcers, arthritis, diabetes, cardiovascular illness and cancer may be caused in part by pathogens. Prevention of infectious diseases and immunotherapeutic treatments of those suffering from infectious diseases, including infections that cause ulcers (Helicobacter pylori), hepatomas (hepatitis virus), hardening of the arteries (Chlamydia pneumoniae), via the development of vaccines would contribute significantly to the length and quality of life, and to a significant reduction of the world's wealth used to provide health care to those afflicted with infectious diseases caused by pathogens.

Many vaccines, especially those containing purified antigenic components or conjugates thereof, are too expensive to have wide utility in the developing world, where economic resources are at a minimum. Such vaccines require refrigeration to maintain potency, which is difficult to provide in tropical parts of the world, and must be administered with syringes that are expensive and require sterilization. Thus, the development of vaccines to be delivered via the mucosal immune system using live carrier bacteria is very important to the public health of the world.

Bacterial Carriers

The use of carrier bacteria, including both gram-negative and gram-positive strains, to deliver vaccine antigens to the mucosal immune system is known to be effective in eliciting cellular and humoral immune responses in both the systemic and mucosal immune systems. Attenuated mucosal pathogenic bacteria, such as Listeria monocytogenes, Salmonella spp., Vibrio cholera, Shigella spp., Mycobacterium bovis BCG, Yersinia enterocolitica and Bacillus anthracis, as well as commensal strains of bacteria, such as Streptococcus gordonii, Lactobacillus spp., Staphylococcus spp., and E. coli have been successfully employed as antigen delivery carriers. For a recent review on the art of using live bacterial carriers, see Medina and Guzman, Vaccine 19:1573-1580 (2001) and Curtiss, Clin. Invest. 110:1061-1066 and references cited therein, which are herein incorporated by reference.

The ability of live recombinant bacteria-based vaccines to colonize the gut-associated lymphoid tissue (Peyer's patches) and the deep tissues following oral administration is beneficial in that it stimulates all arms of the immune response, including mucosal, humoral and cellular immunities (12, 16, 33). Recombinant Salmonella vaccines have also been developed as multivalent vaccines to deliver foreign antigens originating from viruses, bacteria and parasites (12, 33). Orally administered S. typhimurium colonizes the gut-associated lymphoid tissue (Peyer's patches) and the secondary lymphatic tissues including the liver and spleen, to elicit anti-Salmonella immune responses during infection of the mouse (21). The immune responsiveness to orally administered Salmonella has been applied to develop live attenuated oral Salmonella vaccines (12). Attenuated Salmonella vaccines have been constructed by introduction of mutations in the genes required for virulence, including the cyclic AMP receptor protein gene (crp) (11). Crp is a global regulator involved in a variety of biological functions including carbohydrate utilization (4). Attenuated Salmonella vaccine strains have been genetically modified to express heterologous antigens, i.e., antigens that are normally expressed by a different organism, specified by multicopy plasmids. These recombinant vaccines induce immunity to the pathogen whose antigen gene is expressed as well as to Salmonella. It is essential that the antigen specifying plasmids in Salmonella vaccines are stably maintained during the in vivo colonization process. A “balanced-lethal host-vector system” based on the essential bacterial gene aspartate β-semialdehyde dehydrogenase (asd) has been used to specify recombinant antigens from Asd+plasmids that are retained in vivo in asd gene deleted Salmonella vaccine strains (15, 35).

Analysis of convalescent sera from patients or animals infected with bacterial pathogens reveal that the proteins located in the envelopes of or secreted by the bacterial pathogens act as dominant immunogens for the immune responses (30, 37, 56). These observations indicate that envelope and secreted proteins are highly immunogenic and/or more readily interact with antigen presenting cells (APCs) due to their subcellular location. Translocation of such highly immunogenic antigens into the cell envelope or secretion from the cell should increase the strength of the immune response elicited by vaccine strains expressing foreign antigens. In the development of attenuated Salmonella-based multivalent vaccines, a preferable system would use a foreign antigen secreted from the cytoplasm of Salmonella vaccines (18, 19). β-lactamase, encoded by the ampicillin resistance gene, and hemolysin are well-characterized periplasmic secreted proteins in gram-negative bacteria (41, Su et al., 1992, Microb. Pathog. 13:465-476). It is well known that β-lactamase is secreted into the periplasmic space of gram-negative bacteria and its translocation depends upon the presence of a signal sequence consisting of 23 amino acid residues at the N-terminus (25, 41). Evidence obtained from other studies confirms that the signal sequence plus an additional 12 amino acids of the mature β-lactamase are required to translocate β-lactamase through the cytoplasmic membrane of gram-negative bacteria (27, 51). Fusion of a protein to the β-lactamase signal sequence promotes the secretion of the fusion protein into the periplasm of E. coli (40, 51).

Immune Responses

A successfully efficacious vaccine may require that a correct balance is achieved between the different arms of the immune system when an immune response is elicited. Broadly speaking, those arms involve cell-mediated immunity and humoral immunity, which are regulated by helper T cells. Helper T cell-based immune responses are divided into three classes: TH1-, TH2-, and TH0-mediated immune responses. TH1 cells (a) direct cell-mediated immunity, (b) secrete the proinflammatory cytokines gamma-interferon, tumor necrosis factor-beta and lymphotoxin-alpha, and (3) promote class switching to IgG2a. TH1-mediated immunity is generally directed toward intracellular pathogens, including viruses and intracellular bacteria such as Mycobacterium spp. and Salmonella spp. TH1-cells are also involved in inflammation and organ-specific autoimmunity. TH2 cells (a) direct humoral immunity, (b) secrete pro-B-cell cytokines including IL-4, IL-5, IL-9, IL-10, IL-13 and (c) promote class switching to IgG1 and IgA (38, 49). TH2-mediated immunity is generally directed toward extracellular pathogens, including bacteria, helminths and other parasites. TH2-cells are also involved in allergic reactions. TH2-type immune responses are rare in the immune response elicited by attenuated Salmonella vaccines. TH0-cells secrete a combination of TH1 and TH2 cytokines. To date, the mechanism determining TH1- or TH2-type immunity to a given antigen is not well understood. Reviews on TH1- and TH2-mediated immune responses can be found in Golding and Scott, 1995, Ann NY Acad Sci, 754:126-137, Del Prete, G., 1992, Allergy, 47:450-455, and Dong and Flavell, 2000, Arthritis Res, 2:179-188, which are herein incorporated by reference.

Vaccine Directed Against Pneumococcal Antigens

Streptococcus pneumoniae is a human pathogen that causes life-threatening diseases, including community-acquired pneumonia, otitis media, meningitis, and bacteremia in persons of all ages (34). Pneumococcal pneumonia is the leading cause of childhood death worldwide, resulting in over 3 million deaths per year (20). The recent emergence of antibiotic resistant strains has the potential to threaten the treatment of pneumococcal disease in the near future (5). Thus the development of a cost effective pneumococcal vaccine is urgent. Capsular polysaccharide-based pneumococcal vaccines are currently available and are moderately effective. A 23-valent pneumococcal polysaccharide vaccine is recommended for the prevention of infection in adults (46) and a 7-valent conjugated polysaccharide vaccine is licensed for use in children (47). However, vaccination with the pneumococcal polysaccharide vaccine does not reduce the frequency of hospitalization, costs, and mortality caused by pneumococcal pneumonia (22), which reinforces the need for effective new vaccines.

Studies of the protective efficacy of subunit vaccines may further the development of a more protective pneumococcal vaccine. The pneumococcal surface protein A (“PspA”) has been evaluated and considered a pneumococcal vaccine candidate because of its immunogenicity and protection of mice against virulent S. pneumoniae challenge (6, 8, 9, 24). Native PspA_{RX1} (PspA originating from S. pneumoniae Rx1 strain) contains several functional domains: amino terminal signal sequence, alpha-helical region, a proline-rich domain, 10 tandem-repeat choline-binding regions and a 17-amino acid residue carboxy terminus. Pneumococcal protection assays in mice immunized with various recombinant PspA_{RX1} oligopeptides showed that the alpha-helical domain contains the protective epitopes (7). In another study, mice orally immunized with a S. typhimurium vaccine strain expressing a recombinant PspA_{RX1} (from ATG start codon through signal sequence up to the fifth tandem repeat) elicited PspA-specific immune responses and protected against virulent S. pneumoniae challenges (36). Expression of recombinant PspA in this recombinant Salmonella vaccine strain was somewhat toxic such that the high copy number plasmid pYA3193 (pUCori) specifying PspA was relatively unstable. Thus, approximately 50% of cells lost the plasmid after 24 h growth as a standing culture, which is unacceptable for a vaccine or immunogenic composition.

### CITATIONS TO RELATED ART

1. Bertani, G. 1952. Studies on lysogenesis. I. The mode of phage liberation by lysogenic Escherichia coli. J. Bacteriol. 62:293-300.

2. Beveridge, T. J. 1999. Structure of gram-negative cell walls and their derived membrane vesicles. J. Bacteriol. 181:4725-4733.

3. Bolivar, F., R. L. Rodriguez, P. J. Greene, M. C. Betlach, H. L. Heyneker, and H. W. Boyer. 1977. Construction and characterization of new cloning vehicles. II. A multipurpose cloning system. Gene 2:95-113.

4. Botsford, J. L., and J. G. Harman. 1992. Cyclic AMP in prokaryotes. Microbiol. Rev. 56:100-122.

5. Breiman, R., J. C. Butler, F. C. Tenover, J. Elliot, and R. R. Facklam. 1994. Emergence of drug-resistant pneumococcal infections in the United States. JAMA 271:1831-1835.

6. Briles, D. E., J. D. King, M. A. Gray, L. S. McDaniel, E. Swiatlo, and K A. Benton. 1996. PspA, a protection-eliciting pneumococcal protein: immunogenicity of isolated native PspA in mice. Vaccine 14:858-867.

7. Briles, D. E. R. C. Tart, E. Swiatlo, J. P. Dillard, P. Smith, K A. Benton, B. A. Ralph, A. Brooks-Walter, M. J. Crain, S. K. Hollingshead, and L. S. McDaniel. 1998. Pneumococcal diversity: considerations for new vaccine strategies with emphasis on pneumococcal surface protein A (PspA). Clin. Microbiol. Rev. 11:645-657.

8. Briles, D. E., S. K. Hollingshead, G. S. Nabors, J. C. Paton, and A. Brooks-Walter. 2001. The potential for using protein vaccines to protect against otitis media caused by Streptococcus pneumoniae. Vaccine 19:S87-S95.

9. Briles, D. E., S. K. Hollingshead, J. King, A. Swift, P. A. Braun, M. K. Park, L. M. Ferguson, M. H. Nahm, and G. S. Nabors. 2000. Immunization of humans with recombinant pneumococcal surface protein A (rPspA) elicits antibodies that passively protect mice from fatal infection with Streptococcus pneumoniae bearing heterologous PspA. J. Infect Dis. 182:1694-1701.

10. Ciofu, O., T. J. Beveridge, J. Kadurugamuwa, J. Walther-Rasmussen, and N. Hoiby. 2000. Chromosomal β-lactamase is packaged into membrane vesicles and secreted from Pseudomonas aeruginosa. J. Antimicrob. Chemother. 45:9-13.

11. Curtiss, R. III, and S. M. Kelly. 1987. Salmonella typhimurium deletion mutants lacking adenylate cyclase and cyclic AMP receptor protein are avirulent and immunogenic. Infect. Immun. 55:3035-3043.

12. Curtiss, R. 111, T. Doggett, A. Nayak, and J. Srinivasan. 1996. Strategies for the use of live recombinant avirulent bacterial vaccines for mucosal immunization, p.499-511. In H. Kiyono and M. F. Kagnoff (ed.), Essentials of mucosal immunology. Academic Press, San Diego, Calif.

13. Datta, N. and P. Kontomichalou. 1965. Penicillinase synthesis controlled by infectious R factors in Enterobacteriaceae. Nature 208:239-241.

14. Foster, J. W., and B. Bearson. 1994. Acid-sensitive mutants of Salmonella typhimurium identified through a dinitrophenol lethal screening strategy. J. Bacteriol. 176:2596-2602.

15. Gálan, J. E., K Nakayama, and R. Curtiss M. 1990. Cloning and characterization of the asd gene of Salmonella typhimurium: use in stable maintenance of recombinant plasmids; in Salmonella vaccine strains. Gene 94:29-35.

16. Gálan, J. E., and P. J. Sansonetti. 1996. Molecular and cellular bases of Salmonella and Shigella interaction with host cells, p. 2757-2773. In F. C. Neidhardt, R. Curtiss III, J. L. Ingraham, E. C. C. Lin, K. B. Low, B. Magasanik, W. S. Reznikoff, M. Riley, M. Schaechter, and H. E. Umbaeger (ed.), Escherichia coli and Salmonella: cellular and molecular biology, 2^{nd} ed. ASM press, Washington, D.C.

17 Gay, P., D. Le Coq, M. Steinmetz, T. Berkelman, and C. 1. Kado. 1985. Positive selection procedure for entrapment of insertion sequence elements in gram-negative bacteria. J. Bacteriol. 164:918-921.

18. Gentschev, I., H. Mollenkopf, Z. Sololovic, J. Hess, S. H. Kaufmann, and W. Goebel. 1996. Development of antigen-delivery systems, based on the Escherichia coli hemolysin secretion pathway. Gene. 179:133-140.

19. Gentschev, I., I. Glaser, W. Goebel, A J. McKeever, A. Musoke, and V. T. Heussler. 1998. Delivery of the p67 sporozoite antigen of Theileria parva by using recombinant Salmonella dublin: secretion of the product enhances specific antibody responses in cattle. Infect. Immun.66:2060-2064.

20. Greenwood, B. 1999. The epidemiology of pneumococcal infection in children in the developing world. Phil. Trans. R. Soc. Lond. B 354:777-785.

21. Gulig, P. A., and R. Curtiss M. 1987. Plasmid-associated virulence of Salmonella typhimurium. Infect. Immun. 55:2891-2901

22. Hirschmann, J. V. 2000. Use of the pneumococcal polysaccharide vaccine is unwarranted in the U.S. ASM News 66:326-327.

23. Hitchcock, P. J., and T. M. Brown. 1983. Morphological heterogeneity among Salmonella lipopolysaccharide chemotypes in silver-stained polyacrylamide gels. J. Bacteriol. 154:269-277.

24. Hollingshead, S. K., R. Becker, and D. E. Briles. 2000. Diversity of PspA: mosaic genes and evidence for past recombination in Streptocaccus pneumoniae. Infect. Immun. 68:5889-5900.

25. Kadonaga, J. T., A. Pluckthun, and J. R. Knowles. 1985. Signal sequence mutants of lactamase. J. Biol. Chem. 260:16192-16199.

26 Kang, H. Y., C. M. Dozois, S. A. Tinge, T. H. Lee, and R. Curtiss H I. 2001. Transduction-mediated transfer of unmarked deletions and point mutations through the use of counterselectable suicide vectors. J. Bacteriol. (In press)

27. Koshland, D., and D. Botstein. 1980. Secretion of beta-lactamase requires the carboxy end of the protein. Cell 20:749-760.

28. Lennox, E. S. 1955. Transduction of linked genetic characters of the host by bacteriophage P1. Virology 1:190-206.

29. Lo-Man, R., J. P. M. Langeveld, E. Dériaud, M. Jehanno, M. Rojas, J.-M. Clément, R. H. Meloen, M. Hofnung, and C. Leclerc. 2000. Extending the CD4+ T-cell epitope specificity of the Thl immune response to an antigen using a Salmonella enterica serovar Typhimurium delivery vehicle. Infect. Immun. 68:3079-3089.

30. Mathers, K., M. Leinonen, and D. Goldblatt. 1999. Antibody response to outer membrane proteins of Moraxella catarrhalis in children with otitis media. Pediatr. Infect. Dis. J. 18:982-988.

31. McDaniel, L. S., G. Scott, J. F. Kearney, and D. E. Briles. 1984. Monoclonal antibodies against protease sensitive pneumococcal antigens can protect mice from fatal infection with Streptococcus pneumoniae. J. Exp. Med. 160:386-397.

32. McGhee, J. R., and H. Kiyono. 1993. New perspectives in vaccine development: mucosal immunity to infections. Infect. Agents Dis. 2:55-73.

33. Medina, E., and C. A. Guzman. 2001. Use of live bacterial vaccine vectors for antigen delivery: potential and limitations. Vaccine 19:1573-1580.

34. Mufson, M. A. 1990. Streptococcus pneumoniae, p. 1539-1550. In G. L. Mandell, R. G. Douglas, Jr., and J. E. Bennett (ed.), Principles and practice of infectious disease. Churchill Livingstone, Inc., New York, N.Y.

35. Nakayama, K., S. M. Kelly, and R. Curtiss III. 1988. Construction of an Asd⁺ expression-cloning vector: Stable maintenance and high level expression of cloned genes in a Salmonella vaccine strain. Bio/Technology 6:693-697.

36. Nayak, A. R., S. A. Tinge, R. C. Tart, L. S. McDaniel, D. E. Briles, and R. Curtiss III. 1998. A live recombinant avirulent oral Salmonella vaccine expressing pneumococcal surface protein A induces protective responses against Streptococcus pneumoniae. Infect. Immun, 66:3744-3751.

37. Nicolle, L. E., E. Ujack, J. Brunka, L. E. Bryan. 1988. Immunoblot analysis of serologic, response to outer membrane proteins of Escherichia coli in elderly individuals with urinary tract infections. J. Clin. Micicrobiol. 26:2087-2091.

38. O'Garra, A., and N. Aral. 2000. The molecular basis of T helper 1 and T helper 2 cell differentiation. Trends Cell. Biol. 10:542-550.

39. Okahashi, N. M. Yamamoto, J. L. VanCott, S. N. Chatfield, M. Roberts, H. Bluethmann, T. Hiroi, H. Kiyono, and J. R. McGhee. 1996. Oral immunization of interleukin-4 (IL-4) knockout mice with a recombinant Salmonella strain or cholera toxin reveals that CD4⁺ Th2 cells producing IL-6 and IL-10 are associated with mucosal immunoglobulin A responses. Infect. Immun. 64:1516-1525.

40. Pawelek, J. M., K. B. Low, and D. Bermudes. 1997. Tumor-targeted Salmonella as a novel anticancer vector. Cancer Res. 57:4537-4544.

41. Plúckthun, A. and J. R. Knowles. 1987. The consequences of stepwise deletion from the signal-processing site of β-lactamase. J. Biol. Chem. 262:3951-3957.

42. Roland, K., R. Curtiss III, and D. Sizemore. 1999. Construction and evaluation of a Δcya Δcrp Salmonella typhimurium strain expressing avian pathogenic Escherichia coli 078 LPS as a vaccine to prevent airsacculitis in chickens. Avian Dis. 43:429-441.

43. Sambrook, J., E. F. Fritsch, and T. Maniatis. 1989. Molecular cloning, 2^{nd} ed. Cold Spring Harbor Laboratory Press, Cold Spring Harbor, N.Y.

44. Schoöbdel, F., S. M. Kelly, D. L. Peterson, D. R. Milich, and R. Curtiss III. 1994. Hybrid hepatitis B virus core-pre-S proteins synthesized in avirulent Salmonella typhiumurium and Salmonella typhi for oral vaccination. Infect. Immun. 62:1669-1676.

45. Schmieger, H., and H. Backhaus. 1976. Altered cotransduction frequencies exhibited by HT-mutants of Salmonella-phage P22. Mol. Gen. Genet. 143:307-309.

46 Shapiro, E. D., A. T. Berg, R. Austrian, D. Schroeder, V. Pareells, A. Margolis, R. K. Adair, and J. D. Clemmens. 1991. Protective efficacy of polyvalent pneumococcal polysaccharide vaccine. N. Engl. J. Med. 325:1453-1460.

47. Shinefield, H. R., S. Black, P. Ray, 1. Chang, N. Lewis, B. Fireman, J. Hackell, P. R. Paradiso, G. Siber, R. Kohberger, D. V. Madore, F. J. Malinowski, A. Kimura, C. Le, I. Landaw, J. Aguilar, and J. Hansen. 1999. Safety and immunogenicity of heptavalent pneumococcal CRM197 conjugate vaccine in infants and toddlers. Pediatr. Infect. Dis. J. 18:757-763.

48. Singh, S. P., Y. Upshaw, T. Abdullah, S. R. Singh, P. E. Klebba. 1992. Structural relatedness of enteric bacterial porins assessed with monoclonal antibodies to Salmonella typhimurium OmpD and OmpC. J. Bacteriol. 174:1965-1973.

49. Spellberg, B., and J. E. Edwards Jr. 2001. Type I/Type 2 immunity in infectious diseases. Clin. Infect. Dis. 32:76-102.

50. Sternberg, N. L., and R. Maurer. 1991. Bacteriophage-mediated generalized transduction in Escherichia coli and Salmonella typhimurium. Method. Enzymol. 204:18-43.

51. Summers, R. G., and J. R. Knowles. 1989. Illicit secretion of a cytoplasmic protein into the periplasm of Escherichia coli requires a signal peptide plus a portion of the cognate secreted protein. J. Biol. Chem, 264:20074-20081.

52. Talkington, D. F., D. C. Voellinger, L. S. McDaniel, and D. E. Briles. 1992. Analysis of pneumococcal PspA microheterogeneity in SDS polyacrylamide gels and the association of PspA with the cell membrane. Microb. Pathog. 13:343-355.

53. Van den Dobbelsteen, G. P. J. M., and E. P. van Rees. 1995. Mucosal. immune responses to pneumococcal polysaccharides: implications for vaccination. Trends Microbiol. 3:155-159.

54. Witholt, B., M. Boekhout, M. Brock, J. Kingma, H. van Heerikhuizen, and L. de Leij. 1976. An efficient and reproducible procedure for the formation of spheroplasts from variously grown Escherichia coli. Anal. Biochem. 74:160-170,

55. Yamamoto, M., L. S. McDaniel, K. Kawabata, D. E. Briles, R. J. Jackson, J. R. McGhee, and H. Kiyono. 1997. Oral immunization with PspA elicits protective humoral immunity against Streptococcus pneumoniae infection. Infect. Immun. 65:640-644.

56. Yerdugo-Rodriguez, A., L. H. Gam, C. L. Koh, S. D. Puthucheary, E. Calva, and T. Pang. 1993. Detection of antibodies against Salmonella typhi outer membrane protein (OMP) preparation in typhoid fever patients. Asian Pac. J. Allergy. Immunol. 11:45-52.

57. Zhang, X., S. M. Kelly, W. S. Bollen, R. Curtiss III. 1997. Characterization and immunogenicity of Salmonella typhimurium SL1344 and UK-1 Δcrp and Δcdt deletion mutants. Infect. Immun. 65:5381-5387.

### SUMMARY OF THE INVENTION

The inventors have discovered that antigens, which are secreted from a bacterial cell into a vertebrate host cell, stimulate in vertebrates a TH2-type immune response, as ascertained by the detection of IgG1 or IgA antibodies that bind to the secreted antigen, directed toward the secreted antigens. Thus, the present invention is directed to an improved bacteria-based vaccine comprising a plasmid vector that (a) enables the stable expression and secretion of recombinant pathogen antigens, such as PspA, (b) induces a higher immune response to the recombinant pathogen antigen than to endogenous antigens that are expressed by the carrier bacteria, as determined by quantitative analysis of specific antibodies produced by the host vertebrate, and (c) stimulates a TH2-type immune response in the recipient vertebrate. The present invention is drawn to live attenuated pathogenic bacteria (“the carrier bacteria”) that are useful as vaccine carriers, and vaccines and immunogenic compositions that comprise a carrier bacteria. Preferably the bacteria are gram negative bacteria, especially members of the Enterobacteriaceae, Vibrionaceae, Francisellaceae, Legionallales, Pseudomonadacea or Pasteurellaceae groups, including Salmonella spp., Shigella spp., Escherichia spp., Yersinia spp., and Vibrio spp., and the antigen is an antigen derived from a pathogen that is different than the particular strain of carrier bacteria that is used. However, the carrier bacteria may be any bacteria that can infect any one or more of many mucosal membranes, including gut, bronchial, alveolar, conjunctival, and nasal mucous membranes. The carrier bacteria may contain a genetic mutation that results in attenuating the virulence of the carrier bacteria. Attenuating mutations include mutations in the gene encoding cyclic AMP receptor protein (Crp), e.g., Δcrp-28, the gene encoding DNA adenine methylase (Dam), the gene encoding adenylate cyclase (Cya), and the gene encoding β-semialdehyde dehydrogenase (Asd), among others, including for example mutations in the aroA, aroC, aroD, purA, purB, purE, cdt, phoP, phoQ, hemA, ompR, ompc, ompF, rpoS, fur, rfa, rfb, rfc, pmi, galE, and htrA genes.

The carrier bacteria contains a polynucleotide encoding an antigen, which is derived from another pathogen, wherein the antigen is fused to a signal sequence or other secretion peptide, which targets the antigen for secretion from the cytoplasm of the carrier bacteria. In another embodiment, the carrier bacteria may contain two or more polynucleotides, each of which encode an antigen from a different serotype of the same pathogen (such as the PspA antigen from the EF5668 strain and the Rx1 strain of Streptococcus pneumoniae), which are fused to a signal sequence. An object of this multiple antigen embodiment is to provide broad immunological protection to the majority of serotypes of a given pathogen. The antigen (or multiple antigens) is secreted from the bacterial cell into the periplasmic space of the bacterium or into the surrounding environment, which may be the interior of the host vertebrate cell or the interstitial space outside of the host cell, but within the host tissues. When the vaccine, immunogenic composition or carrier bacteria is administered to a vertebrate, the antigen is secreted from the carrier bacteria and the vertebrate elicits a Th2 response toward the secreted antigen. Preferred vertebrates are humans, livestock, poultry, and companion animals. Preferred pathogens from which the antigen is derived, especially when the vertebrate is a human, include Streptococcus pneumoniae., Streptococcus mutans, Streptococcus sobrinus, Streptococcus equi, Erysipelothrix rhusiopathiae, Mycobacterium tuberculosis, Listeria spp., Bacillus anthracis, Clostridium spp., and Corynebacterium spp. The preferred antigen is a pneumococcal surface protein A (“PspA”), which sequence is set forth in SEQ ID NO:1 or SEQ ID NO:26, or a fragment of SEQ ID NO:28. The preferred secretion peptide is a fragment of the β-lactamase enzyme, which fragment sequence is set forth in any one of SEQ ID NO:2 or SEQ ID NO:3, or a portion of SEQ ID NO:28.

The carrier bacteria may comprise a balanced-lethal host system to help maintain the polypeptide, which encodes the secretion competent antigen, in the carrier bacteria. The preferred balanced lethal host system relies on the complementation of a lethal mutation (ΔasdA16) in the chromosomal gene encoding aspartate β-semialdehyde dehydrogenase (Asd). Asd is required for the synthesis of diaminopimelic acid (“DAP”), an essential component of the rigid layer of the bacterial cell wall. The polynucleotide encoding the secretion competent antigen is placed onto a plasmid that contains a functional copy of a gene encoding Asd. Thus, in order for the carrier bacteria to survive, it must maintain the plasmid, which contains the polynucleotide encoding the secreted antigen.

The carrier bacteria may comprise an environmental limitation viability system, which ensures that the carrier bacteria will not survive in the environment outside of the vertebrate to which the carrier bacteria was administered. For example, the carrier bacteria may contain a mutation in an essential gene, wherein the essential gene is complemented by a regulatable copy of the essential gene, such that a functional gene product is expressed only at the temperature found within a vertebrate; or the carrier bacteria may contain a gene that encodes a polypeptide that is toxic to the carrier bacteria, wherein the toxic polypeptide is produced only at the temperature outside of the vertebrate. Alternative biological containment systems are described in U.S. provisional patent application serial No. 60/407,522 and U.S. patent application entitled “Regulated Attenuation of Live Vaccines to Enhance Cross-Protective Immunogenicity. Filed Apr. 15, 2003, both of which are hereby incorporated by reference.

The invention is also drawn to methods of eliciting an immune response in a vertebrate, wherein the carrier bacteria, as described in the preceding paragraphs, is administered to the vertebrate. The antigen, which is derived from a pathogen that is different than the carrier bacteria, is secreted from the carrier bacteria and the vertebrate produces IgG1 antibodies that specifically bind to the antigen. Preferably the vertebrate is a human, livestock, poultry or companion pet. Preferably the antigen is derived from a pathogen such as Streptococcus pneumoniae, Streptococcus mutans, Streptococcus sobrinus, Streptococcus equi, Erysipelothrix rhusiopathiae, Mycobacterium tuberculosis, Listeria spp., Bacillus anthracis, Clostridium spp., and Corynebacterium spp.

### BRIEF DESCRIPTION OF THE DRAWINGS

FIG. 1: Reduced expression of Asd protein by deletion of asd gene promoter region. SDS-PAGE was performed with cell lysates of S. typhimurium×4550 with pYA3333 (entire asd gene with P_{asd}, pBRori), pYA3334 (entire asd gene P_{asd}, pUCori), pYA3342 (promoterless SD-asd gene, pBRori) and pYA3341 (promoterless SD-asd gene, pUCori). Standards are indicated to the left and Asd protein (39 kDa) is designated by arrow. Lanes; 1, pYA3333; 2, pYA3334; 3, pYA3342; 4, pYA3341.

FIG. 2: Asd⁺ antigen expression vectors. (A) Asd⁺ vector pYA3342. The map of pYA3342 and the nucleotide sequences of the P_{trc}, promoter region and multicloning sites are depicted. (B) Periplasmic secretion Asd⁺ vector pYA3493. A DNA fragment encoding the β-lactamase signal sequence and 12 amino acid residues of the N-terminus of mature β-lactamase of plasmid pBR322 (SEQ ID NO:2) was positioned under the control of the P_{trc} promoter of an Asd⁺ vector pYA3342 (pBRori). The map of pYA3493 and the nucleotide sequences of the P_{trc} promoter region, β-lactamase signal sequence (bla SS) and multicloning sites are depicted. The P_{trc} sequences for -35, -10, Shine-Dalgarno (SD), and translation start codon are bold typed. An arrow within the sequence indicates the signal peptidase cleavage site. Unique restriction enzyme sites in the multicloning site are indicated. 5ST1T2 is a transcriptional terminator.

FIG. 3: Recombinant plasmid pYA3494 for PspA overexpression. (A) PspA region used in this study. Functional domains of native PspA from S. pneumoniae (PspA) are diagramed; dotted box, leader sequence (31 aa); open box, immuno dominant a-helical region (1-288 aa); box with slanted lines, proline-rich region (289-370 aa); ten gray boxes, choline-binding repeats (371-571 aa); black box, C-terminus (572-588 aa). Dotted lines represent the limit of the recombinant PspA (rPspA) region used in this study. Bioinformatical analyses of the PspA for antigenic index and surface probability are presented. Analyses were performed with the Protean module of the Lasergene sequence analysis software. (B) The map of recombinant plasmid pYA3494. A 0.7 kb EcoRI-Hind III fragment of PCR amplified DNA fragment of pspAᵣₓ₁ was cloned into the EcoRI and HindIII sites of pYA3493 (FIG. 2B). The cloned fragment included the immunogenic α-helical region of PspA including amino acids 3 through 257 of mature PspA (255 amino acids).

FIG. 4: Subcellular location of expressed rPspA in S. typhimurium. Subcellular fractions were prepared from S. typhimurium×8599 (pYA3494) cells grown in LB broth at 37° C. Fractions equivalent to 30 μl volume of 0.8 OD₆₀₀ culture except for supernatant fluid were analyzed by SDS-PAGE and the rPspA was detected by immunoblot with PspA specific monoclonal antibody Xi126. β-galactosidase and OmpC were used as fractionation controls for cytoplasmic and outer membrane fractions, respectively. Standards are indicated to the left. Lanes; 1, total cell lysate; 2, cytoplasm; 3, periplasm; 4, outer membrane; 5, concentrated supernatant (750 μl); 6, supernatant (10 μl).

FIG. 5: Expression of rPspA in the S. typhimurium vaccine strain. ×8501 harboring pYA3494 (specifying rPspA) or pYA3493 (vector control) was cultured in LB broth at 37° C. Total cells (equivalent to 7.5×10⁸ cells) and concentrated culture supernatants (equivalent to 750 μl of supernatant of 0.8 OD₆₀₀ culture) were subjected to SDS-PAGE analysis. The left panel is a Coomassie brilliant blue stained gel. The right panel is an immunoblot of the duplicated gel with PspA specific monoclonal antibody Xil26. Molecular markers are indicated to the left. PspA proteins are indicated. Lanes 1 and 2 represent protein profiles of ×8501 (pYA3493) and ×8501 (pYA3494), respectively.

FIG. 6: Serum IgG responses to S. typhimurium LPS and SOMPs, and foreign antigen rPspA. The data represent IgG antibody levels, as determined by ELISA, induced in mice orally immunized with ×8501 (pYA3493) (vector control) and ×8501 (pYA3494) (expressing rPspA) at designated weeks after immunization. Black arrows indicate sublethal i.v, infection with S. pneumoniae WU2. Columns; 1, 2 weeks; 2, 4 weeks; 3, 6 weeks; 4, 8 weeks; 5, 10 weeks; 6, 12 weeks; 7, 17 weeks; 8, 19 weeks, 9, 21 weeks.

FIG. 7: Secretory IgA responses to S. typhimurium LPS and SOMPs, and recombinant PspA. The data represent anti-LPS, -SOMPs and -rPspA IgA antibody levels in vaginal secretions of BALB/c mice orally immunized with ×8501 (pYA3493) (vector control) and ×8501 (pYA3494) (expressing rPspA) at weeks 4, 6, 8, and 10 after immunization.

FIG. 8. Serum IgG2a and IgG1 responses to S. typhimurium LPS and SOMPs, and recombinant PspA. The data represent IgG2a and IgG1 subclass antibody levels to Salmonella LPS and SOMPs and rPspA in sera of BALB/c mice orally immunized with ×8501 (pYA3493) (vector control) and ×8501 (pYA3494) (expressing rPspA) at designated weeks after immunization. Black arrows indicate sublethal i.v. infection with S. pneumoniae WU2. Anti-rPspA IgG2a and IgG1 responses of ×8501 (pYA3493) (negative control) were not shown. Columns; 1, 2 weeks; 2, 4 weeks; 3, 6 weeks; 4, 8 weeks; 5, 10 weeks; 6, 12 weeks; 7, 17 weeks; 8, 19 weeks; 9, 21 weeks.

FIG. 9. Relationship between PspA amino acid sequences. This figure diagrammatically depicts the familial relationship among the PspA proteins in S. pneumoniae of diverse capsular polysaccharide serotypes.

FIG. 10 depicts the nucleotide sequence (SEQ ID NO:13) and protein sequence (SEQ ID NO:26) for the N-terminal portion of the EF5668 pspA gene including the signal sequence and the α-helical domain.

FIG. 11 lists all the oligonucleotide primers to first clone the EF5668 α-helical domain into pYA3493 to yield pYA3594. Primers 1 through 6 are SEQ ID NO:14, SEQ ID NO:15, SEQ ID NO:16, SEQ ID NO:17, SEQ ID NO:18 and SEQ ID NO:19, respectively.

FIG. 12 depicts the stepwise construction of plasmid pYA3605.

FIG. 13 depicts the polynucleotide encoding the β-lactamase/PspA_{EF5668}/PspA_{Rx1} fusion protein (SEQ ID NO:20), and the conceptually translated polypeptide sequence (SEQ ID NO:28).

FIG. 14. Western blot analysis of β-lactamase/PspA_{EF566}/PSPA_{Rx1} fusion protein expression by ×6212. Monoclonal antibodies directed against both PspA_{EF5668} and PspA_{Rx1} antigens were each able to detect the fusion protein, which was of the expected size.

FIG. 15 depicts the stepwise construction of plasmid pYA3620.

FIG. 16 depicts the polynucleotide sequence of β-lactamase signal sequence (bla SS) and carboxyterminal (bla CT) sequence as in plasmid pYA3620 (SEQ ID NO:25), and the corresponding conceptually translated polypeptide sequences.

FIG. 17 depicts the IgG immune responses to LPS and PspA induced by Salmonella vaccine strain ×8501 comprising pYA3494, which encodes secretion competent PspA, and Salmonella vaccine strain ×8501 comprising pYA3496, which encodes cytoplasmic PspA.

FIG. 18 shows the pspA sequence from S. pneumoniae Rx1.

FIG. 19 shows the DNA and amino acid sequences of the codon optimized pSpA_{Rx1} with optimized codons indicated in bold-face type.

FIG. 20 shows the DNA and amino acid sequences of the codon optimized pSPA_{EF5668} with optimized codons indicated in bold-face type.

FIG. 21 shows the DNA and amino acid sequence of the codon optimized pspA_{EF5668-Rx1} fusion with optimized codons indicated in bold-face type.

FIG. 22 is a list of primer used for constructions shown in FIGS. 19, 20 and 23.

FIG. 23 illustrates contruction of pspA plasmids using oligonucleotide sequences shown in FIG. 22.

FIG. 24 shows vectors with original pneumococcal and codon optimized sequences for PSPA_{Rx1}, PSPA_{EF5668} and the PspA_{EF5668-Rx1} fusion.

FIG. 25 illustrates construction of pYA3637 with the codon optimized PSPA_{EF5668} sequence inserted into the pYA3620 expression vector.

FIG. 26 shows the DNA and amino acid sequences of the bla SS-pspA_{EF5668}-bla C-terminus region in pYA3637.

FIG. 27 illustrates the expression of PspA_{EF5668}, PspA_{Rx1} and fPspA (PspA_{Ef5668-Rx1}) in Salmonella.

FIG. 28 shows the pYA3332 Asd⁺ vector with pl5A ori.

FIG. 29 shows Salmonella chromosomal insertion-deletion and deletion mutations.

FIG. 30 shows suicide vectors for introducing the ΔilvG3::TT araC P_{BAD}/ac/TT, ΔaraBAD23, and ΔaraE25 mutations.

FIG. 31 shows the DNA and amino acid sequences of pspA from S. pneumoniae 6B.

### DETAILED DESCRIPTION OF THE INVENTION

The invention is directed to an improved vaccine or immunogenic composition that delivers an antigen to the mucosal immune system of a vertebrate and provokes a Th2-mediated immune response in the recipient vertebrate against the antigen. The invention is also directed to methods of eliciting a Th2-mediated immune response in a vertebrate, comprising administering a vaccine or immunogenic composition to the vertebrate. The vaccine or immunogenic composition comprises a live attenuated strain of pathogenic bacteria, wherein the strain of bacteria is capable of infecting or colonizing any one or more mucosal tissues within the vertebrate. As used herein, the term “attenuated” means that the carrier bacteria is less virulent than the wild-type version of the bacteria from which the carrier is derived. The term “virulence” means the ability of a pathogen to cause disease. Preferred vertebrates include humans, livestock, poultry and companion pets.

The mucosal immune system comprises several components, including the nasal-associated lymphoid tissue (“NALT”), bronchus-associated lymphoid tissue (“BALT”), gut-associated lymphoid tissue (“GALT”), intestinal epithelial cells (“IEC”), lamina propria, dendritic cells, mucosal macrophages, and the like. For a detailed discussion of the mucosal immune system, see Mucosal Immunology 2^{nd} edition, edited by Ogra, P. L. et al., Academic Press, San Diego, 1999, which is herein incorporated by reference.

The improved vaccine or immunogenic composition comprises a live attenuated strain of a pathogenic bacteria capable of infecting any one component or several components of the mucosal immune system. The live attenuated strain of pathogenic bacteria, also referred to as the “carrier”, contains a polynucleotide that encodes an antigen derived from a pathogen that is different than the carrier bacteria, wherein the antigen is secreted from the carrier bacteria. The carrier bacteria may also comprise multiple polynucleotides, each of which encodes an antigen from a different serotype of the same pathogen (that is different than the carrier bacteria). As used herein, the term “serotype” or the phrase “different serotype of the same pathogen” means a closely related strain, usually a strain of bacteria, found within the same species. For example, Salmonella typhi and Salmonella typhimurium are different serotypes of the species Salmonella enterica. In another example, Streptococcus pneumoniae strain EF5668 and Streptococcus pneumoniae strain Rx1 are different serotypes of the species Streptococcus pneumoniae. The term “serotype” is a term of art.

Methods of making and using live attenuated strains of bacteria that are suitable for vaccines or immunogenic compositions, including instruction on how to make mutations in virulence genes, are taught in U.S. Pat. Nos. 5,294,441, 5,387,744, 5,389,368, 5,468,485, 5,855,879, and 5,855,880, which are herein incorporated by reference. Those patents teach methods of attenuating pathogenic bacteria to be used as vaccine or immunogenic composition carriers. Examples of genes that may be mutated to confer attenuation include cya (which encodes adenylate cyclase), crp (which encode cAMP receptor protein), asd and dam (which encodes DNA adenine methylase). The role of mutations in the dam gene is discussed in Fricker, J., “New vaccines: damming for multi-strain organisms?” Drug Discovery Today 7:212-213 (2002) and all references cited therein, which are herein incorporated by reference. In a preferred embodiment of the invention, the live attenuated carrier bacteria is rendered attenuated by having an inactivating mutation in the crp gene.

The vaccine, immunogenic composition or carrier bacteria may comprise a “balanced-lethal host-vector system”, which enables the carrier bacteria to maintain the presence of the ectopic polynucleotide constructs without the need for external selection. How to make and use the “balanced-lethal host-vector system” are taught in U.S. Pat. Nos. 5,294,441, 5,387,744, 5,424,065, 5,656,488, 5,672,345, 5,840,483, 5,855,879, 5,855,880 and 6,024,961, and PCT/US01/13915, which are herein incorporated by reference. The balanced-lethal host-vector system is based upon the concept of having an inactivating mutation in an essential gene of the carrier bacteria, wherein a functional copy of the essential gene is provided on a plasmid (a genetically engineered autonomous extrachromosomal polynucleotide), which contains the polynucleotide that encodes the antigen. Thus, in order for the carrier bacterium to remain viable, the plasmid, which contains the functional copy of the essential gene and the polynucleotide that encodes the antigen, must be maintained in the carrier bacterium. This ensures that the polynucleotide that encodes the antigen is maintained in the carrier bacterium. In a preferred embodiment of the invention, the essential gene encodes β-aspartate semialdehyde dehydrogenase (Asd), and the plasmid encodes a functional Asd polypeptide, which complements the chromosomal asd mutation, but which cannot replace the defective chromosomal Asd gene by recombination. Lack of a functional Asd polypeptide causes bacterial cells to lyse. The polynucleotide encoding the functional Asd polypeptide and the polypeptide that encodes the antigen are physically linked on the same episome, thereby ensuring that the carrier bacteria maintains the polynucleotide that encodes the antigen.

The vaccine or immunogenic composition may also comprise an “environmental limited viability system” to prevent the survival of, or to kill those carrier bacteria that escape into the environment. How to make and use the “environmental limited vaccine system” are taught in copending U.S. patent applications Ser. Nos. 08/473,789 and 08/761,769, which are herein incorporated by reference. Alternative biological containment systems are described in U.S. provisional patent application serial No. 60/407,522 and U.S. patent application entitled “Regulated Attenuation of Live Vaccines to Enhance Cross-Protective Immunogenicity,” filed Apr. 15, 2003, both of which are hereby incorporated by reference.

The inventors have discovered that a Th2-mediated immune response is elicited toward antigens that are secreted by the carrier bacteria, as compared to antigens that are not secreted by the carrier bacteria. To allow for the secretion of the antigen from the carrier, the polynucleotide encoding the antigen is operably linked to a second polynucleotide sequence that encodes a peptide that enables the secretion of the antigen from the carrier. A subset of the peptides that enable the secretion of antigens and other polypeptides from a cell are known in the art as signal sequences. As used herein, the term “secretion peptide” means any peptide, which when fused to any polypeptide facilitates the secretion of that polypeptide from a cell. Any secretion peptide may be used in the practice of the instant invention. In a preferred embodiment of this invention, the secretion peptide comprises a fragment of a β-lactamase polypeptide. Most preferably, the secretion peptide comprises (a) the 35 amino-terminal amino acids of the precursor β-lactamase polypeptide as set forth in SEQ ID NO:2 or (b) the 21 carboxy-terminal amino acids of β-lactamase as set forth in SEQ ID NO:3. However, the skilled artisan in the practice of this invention may substitute any peptide sequence as a secretion peptide, as long as the substitute peptide facilitates or enables the secretion of the antigen from the carrier bacteria, is stable, and is non-toxic to the vertebrate or bacterial host. Preferred secretion peptides are derived from gram negative bacteria.

Carrier bacteria that are applicable to the operation of this invention may be selected on the basis of their ability to infect or colonize specific body sites. However, it has been demonstrated that gut pathogen-based vaccine carriers, such as attenuated Salmonella, are capable of delivering antigens to several mucosal sites in addition to GALT, including nasal, vaginal, oral and rectal. Therefore, as is shown in the examples that follow, administration to the GALT of an antigen of a respiratory pathogen will result in protective immunity toward the respiratory pathogen. Thus the skilled artisan would reasonably expect the administration of an Enterobacteriaceae carrier, for example, that expresses a respiratory pathogen antigen to be effective in eliciting a protective immune response against the respiratory pathogen.

Carrier bacteria and antigens are also selected on the basis of the nature of the intended recipient vertebrate. For humans, the carrier bacterium may be a Vibrio cholera, Shigella spp., Yersinia spp., or any one of Salmonella enteritidis serotypes, such as Salmonella typhi or Salmonella paratyphi A, B or C. For animals, the carrier bacteria may be, for example, Salmonella strains such as Salmonella choleraesuis for swine, S. dublin for cattle, S. abortusovis for sheep, or S. gallinarum for poultry. Given the fact that the mucosal vaccination art is replete with methods of delivering antigens to mucosal surfaces, the skilled artisan will readily recognize that other bacterial carriers may be used in the practice of this invention. Strains of bacteria, which may be suitable in the practice of this invention, include Salmonella spp., Vibrio cholerae, Yersinia enterocolitica, Shigella spp., and Escherichia coli, and hybrids thereof. These and other carrier bacteria, as well as pathogens toward which the vaccine or immunogenic composition may be directed, are taught in U.S. Pat. Nos. 4,888,170, 5,110,588, 5,389,368, 5,468,485, 5,888,799 and 6,024,961, and art recognized publications, including Velge-Roussel et al., Infect Immun 68:969-972 (2000), Kyd and Cripps, Vaccine 17:1775-1781 (1999), Husband, Vaccine 11:107-112 (1993), Mestecky and McGhee, Adv Exp Med Biol 327:13-23 (1992), Langermann et al., Nature 372:552-555 (1994), Rush et al., Adv Exp Med Biol 371 B: 1547-1552 (1995), Sizemore et al., Vaccine 15:804-807 (1997), Robinson et al., Nat Biotechnol 15:653-657 (1997), Shaw et al., Immunology 100:510-518 (2000), Thole et al., Curr Opin Mol Ther 2:94-99 (2000), Lee at al., Vaccine 19:3927-3935 (2001), Medina and Guzman, Vaccine 19:1573-1580 (2001), Wells et al., Antonie Van Leeuwenhoek 70:317-330 (1996), which are herein incorporated by reference.

Pathogens to which the vaccine or immunogenic composition is directed include worms and other helminths, fungi, viruses, protozoans, neoplastic cells, and bacteria. While gametes are not pathogens, it is envisioned that the vaccine or immunogenic composition of the instant invention may be directed toward gametes and thus may be used as a birth control method or anti-fertility treatment. The antigen to be expressed and secreted by the carrier bacteria is selected from the pathogen to which the vaccine or immunogenic composition is directed. For a highly specific or narrow spectrum vaccine or immunogenic composition, the antigen preferably comprises an epitope that is found only in a particular pathogen or serotype thereof. Preferably, to make an effective vaccine, such an epitope is universally recognized. For a general or broad spectrum vaccine or immunogenic composition, the antigen preferably comprises an epitope or group of epitopes that is found in several pathogens or serotypes thereof. Also, broad spectrum vaccines or immunogenic compositions may ectopically express multiple antigens that are shared among different pathogens. Preferred pathogens, include Clostridium spp., Corynebacterium spp., Bacillus anthracis, Streptococcus pneumoniae, Streptococcus mutans, Streptococcus sobrinus, Streptococcus equi, Streptococcus pyogenes, Erysipelothrix rhusiopathiae, and Mycobacterium tuberculosis.

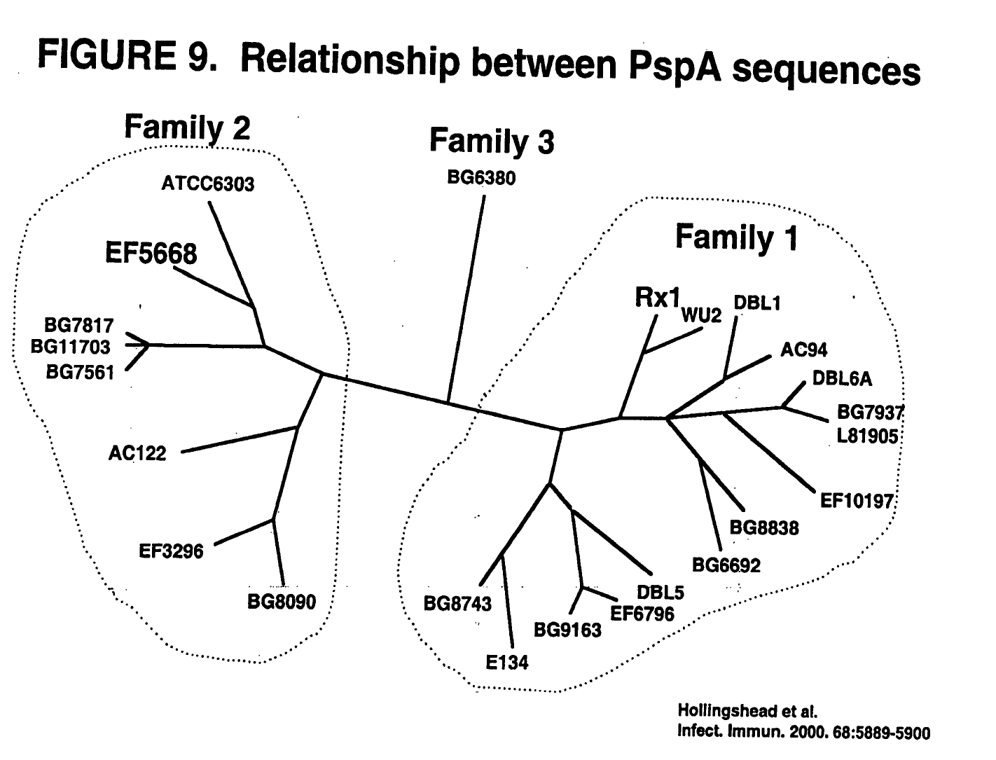

In a preferred embodiment of this invention, the secretion-competent antigen comprises a polypeptide fragment of the pneumococcal surface protein A (“PspA”) fused to the β-lactamase signal sequence plus the first 12 amino acids of the secreted from of β-lactamase. The fragment of the PspA may comprise the alpha helical domain of Streptococcus pneumoniae strain EF5688 (SEQ ID NO:1, amino acid residues 110-384 of SEQ ID NO:26), the alpha helical domain of Streptococcus pneumoniae strain Rxl, or both. As discussed in Example 5, the strain EF5688 represents one major family of Streptococcus pneumoniae, while strain Rx1 represents the other major family of Streptococcus pneumoniae (see FIG. 9.) Therefore, the combination of PspA fragments from both strain EF5688 and strain Rx1 is expected to provide an antigen that elicits an antibody immune response that would be protective against infection with the greatest diversity of Streptococcus pneumoniae capsular polysaccharide serotype strains. A preferred secretion-competent antigen comprises a sequence as set forth in SEQ ID NO:25. Other preferred antigens include codon-optimized sequences encoding PspA, as described in Example 8, and an alpha-helical domain of PsaA antigen as described in Example 12.

The vaccine or immunogenic composition of the instant invention may be administered to vertebrates, which include humans as well as companion pets, vermin, livestock and poultry according to several methods. In order for a vaccine to be effective in stimulating a Th2-mediated immune response, the antigenic materials must be released or presented in such a way to trigger the induction of memory T-cells or other CD4⁺ T-cells directed at the Th2 arm of the immune system of the vaccinated vertebrate. Therefore, the carrier bacteria comprising of the polypeptide that encodes the secretion-competent antigen must be introduced into the vertebrate. In order to stimulate a preferred response of the gut-associated lymphoid tissue (GALT) or bronchus-associated lymphoid tissue (BALT), introduction of the vaccine or immunogenic composition directly into the gut or bronchus is preferred, such as by oral administration, gastric intubation or intranasally in the form of aerosols, although other methods of administering the vaccine, such as intravenous, intramuscular or subcutaneous injection, or intramammary, intrapenial, vaginal or rectal administration, are possible.

Administration of a live vaccine of the type disclosed above to an individual can be by any known technique. These include oral ingestion, gastric intubation, broncho-nasal-ocular spraying, or whole-body spray. The whole-body spray method of administering a vaccine or immunogenic composition, wherein droplets comprising the attenuated carrier bacteria are delivered as coarse droplets to the body of the recipient vertebrate, is described in detail in the PCT publication WO 00/04920, which is herein incorporated by reference. All of these methods allow the live vaccine to easily reach the GALT or BALT cells and induce antibody formation and cell mediated immunity. Other methods of administration, such as intravenous injection, that allow the carrier microbe to reach the individual's blood stream can be acceptable. Intravenous, intramuscular or intramammary injection are also acceptable with other embodiments of the invention. The immunization dosages required will vary with the antigenicity of the gene product and need only be an amount sufficient to induce an immune response. Routine experimentation will easily establish the required amount. Multiple dosages are used as needed to provide the desired level of protection.

The pharmaceutical carrier or excipient in which the vaccine is suspended or dissolved may be any solvent or solid or encapsulating material such as for a lyophilized form of the vaccine. The carrier is non-toxic to the vertebrate and compatible with the carrier bacteria and antigenic gene product. Suitable pharmaceutical carriers are known in the art and, for example, include liquid carriers, such as normal saline and other non-toxic salts at or near physiological concentrations, and solid carriers, such as talc or sucrose. Gelatin capsules can serve as carriers for lyophilized vaccines. Adjuvants may be added to enhance the antigenicity if desired, but are generally not required to induce an effective immune response, since the bacterial carriers themselves generally serve as adjuvants. When used for administering via the bronchial tubes, the vaccine is preferably presented in the form of an aerosol. Suitable pharmaceutical carriers and adjuvants and the preparation of dosage forms are described in, Remington's Pharmaceutical Sciences, 17th Edition, (Gennaro, Ed., Mack Publishing Co., Easton, Pa., 1985), which is herein incorporated by reference.

Immunization of a vertebrate with an antigen derived from a pathogen can also be used in conjunction with prior immunization with the carrier bacteria, which expresses the antigen derived from the pathogen. Such parenteral immunization can serve as a booster to enhance expression of the Th2-mediated immune response. The enhanced response is known as a secondary, booster, or anamnestic response and results in prolonged immune protection of the host vertebrate. Booster immunizations may be repeated numerous times with beneficial results.

The above disclosure describes several preferred embodiments of the invention. The skilled artisan will recognize that other embodiments of this invention, which are not overtly disclosed herein, may be employed in the practice of this invention. The invention is further illustrated by the examples described below, which are not meant to limit the invention.

### EXAMPLES

For ease of reading, citations to related art documents are indicated as numbers in parentheses. Those numbered citations refer to the bibliography in the section “Background of the Invention”, subsection “Citations to related art”. Those related art documents have been incorporated by reference.

TABLE 1
Bacterial strains and plasmids

| | | Derivation, |
|---|---|---|
| | | source or |
| Strain or plasmid | Relevant characteristics^{a} | reference |
| Strains | | |
| E. coil | | |
| χ6212 | F φ80d IacZ ΔM15 deoR Δ(IacZYA-argF)U169 supE44 | 35 |
| | λgyrA96 recA1 relA1 endA1 ΔasdA4 Δzhf-2::Tn10 | |
| | hsdR17(R⁻M⁺) | |
| MGN-617 | thi-1 thr-1 leuB6 fhuA21 lacY1 gln V44 ΔasdA4 recA1 | 42 |
| | rp4 2-Tc::Mu (λpir), Km^{r} | |
| S. typhimurium | | |
| χ3339 | SL1344 hisG | 21 |
| χ3761 | UK-1 wild-type | Curtiss et. al. 1991 |
| χ4550 | SR-11 gyrA1816 Δcrp-1 ΔasdA1 Δ(zhf-4::Tn10) Δcya-1 | 44 |
| χ4746 | nadA540::Tn10Δ(galE-ch1-uvrB)-1005, Tet^{r} | χ3339 |
| χ8477 | UK-1 ΔaraE25 | χ3761 |
| χ8449 | hisG Δcrp-28 | χ3339 |
| χ8501 | hisG Δcrp-28 ΔasdA16 | χ8449 |
| χ8554 | hisG ΔasdA16 | χ3339 |
| χ8599 | hisG ΔasdA16 atrB13::MudJ | χ8554 |
| χ8623 | UK-1 ΔilvG3::TT araC P_{BAD} lacl | χ3761 |
| χ8767 | UK-1 ΔaraBAD23 | χ3761 |
| JF2430 | LT-2 atrB13::MudJ | 14 |
| S. paratyphi A | | |
| χ8219 | wild type, ATCC#9281 | ATCC |
| χ8387 | Pla | χ8219 |
| χ8488 | ΔphoP24 | χ8387 |
| χ8523 | ΔphoP24 ΔasdA16 | χ8488 |
| χ8694 | ΔasdA25 | χ8387 |
| S. typhi | | |
| χ8438 | Ty2 wild-type, RpoS⁺, ATCC#202182 | Curtiss collection |
| χ8542 | ΔphoP24 | χ8438 |
| χ3744 | ISP1820 wild-type | Curtiss collection |
| χ8521 | ΔphoP24 | χ3744 |
| χ8522 | ΔphoP24 ΔasdA16 | χ8521 |
| S. pneumoniae | | |
| WU2 | wild-type virulent, encapsulated type 3, PspA type 1 | 6 |
| EF5668 | Capsular type 4, PspA type 12 | McDaniel et. al. |
| | | 1992 |
| Rx1 | Nonencapsulated avirulent, highly transformable variant | McDaniel et. al. |
| | of D39, PspA type 25 | 1986 |
| 6B | Capsular type | Sampson et. al. |
| | | 1997 |
| Plasmids | | |
| pYA3193 | Asd⁺vector harboring 1.5 kb C-terminally truncated | 36 |
| | pspA gene, pUC ori | |
| pYA3332 | Asd⁺, p15A ori | Curtiss collection |
| pYA3333 | Asd⁺, pBR ori | Curtiss collection |
| pYA3334 | Asd⁺, pUC ori | Curtiss collection |
| pYA3341 | SD-Asd⁺, pUC ori | Curtiss collection |
| pYA3342 | SD-Asd⁺, pBR ori | Curtiss collection |
| pYA3485 | Suicide vector to introduce ΔaraE25 mutation, Cm^{r} | Curtiss collection |
| pYA3493 | pYA3342 derivative β-lactamase signal sequence- | Curtiss collection |
| | based periplasmic secretion plasmid | |
| pYA3494 | 0.7 kb DNA encoding the α-helical region of PspA in | Curtiss collection |
| | pYA3493 | |
| pYA3496 | 0.7 kb DNA encoding the α-helical region of PspA in | Curtiss collection |
| | pYA3342 for the expression of His-tagged PspA | |
| pYA3599 | Suicide vector to introduce ΔaraBAD23 mutation, Cm^{r} | Curtiss collection |
| pYA3605 | pYA3494 derivative harboring pspA_{EF5668-Rx1} | Curtiss collection |
| pYA3620 | pYA3342 derivative harboring β-lactamase signal | Curtiss collection |
| | sequence and C-terminal sequence-based | |
| | periplasmic secretion plasmid | |
| pYA3623 | pYA3494 derivative harboring pspA_{EF5668} | Curtiss collection |
| pYA3633 | pYA3494 derivative harboring codon optimized | Curtiss collection |
| | pspA_{EF5668} sequence | |
| pYA3634 | Replacing pYA3494 in which base G is deleted at | Curtiss collection |
| | mature amino acid 234. | |
| pYA3635 | pYA3494 derivative harboring codon optimized | Curtiss collection |
| | pspA_{Rx1} sequence | |
| pYA3636 | pYA3494 derivative harboring codon optimized | Curtiss collection |
| | pspA_{EF5668-Rx1} sequence | |
| pYA3637 | pYA3620 derivative harboring codon optimized | Curtiss collection |
| | pspA_{EF5668} sequence | |
| pBR322 | Cloning vector, Ap^{r}, Cm^{r} | 3 |
| pMEG-249 | Suicide vector to generate Salmonella ΔilvG3::TT | Megan Health Inc |
| | araC P_{BAD} lacl mutant, Ap^{r}, Cm^{r} | |
| pMEG-443 | Suicide vector to generate Salmonella ΔasdA16 | 26 |
| | mutant, Ap^{r}, Cm^{r} | |
| pMEG-493 | Suicide vector to generate Salmonella Δcrp-28 | Megan Health Inc |
| | mutant, Ap^{r}, Cm^{r} | |

^{a}Ap ampicillin; Cm, chloramphenicol; Km, Kanamycin; Tc, tetracycline.

### REFERENCES

Curtiss III, R., S. B. Porter, M. Munson, S. A. Tinge, J. O. Hassan, C. Gentry-Weeks, and S. M. Kelly. 1991. Nonrecombinant and recombinant avirulent Salmonella live vaccines for poultry colonization, p.169-198. In Control of Human Bacterial Enteropathogens in Poultry. Academic Press, Inc., N.Y.

McDaniel L S, Scott G, Widenhofer K, Carroll J M, Briles D E. 1986. Analysis of a surface protein of Streptococcus pneumoniae recognised by protective monoclonal antibodies. Microbial Pathog. 1:519-31.

McDaniel, L. S., J. S. Sheffield, E. Swiatlo, J. Yother, M. J. Crain, and D. E. Briles. 1992. Molecular localization of variable and conserved regions of pspA, and identification of additional pspA homologous sequences in Streptococcus pneumoniae. Microbial Pathog. 13:261-269.

Sampson, J. S., Z. Furlow, A. M. Whitney, D. Williams, R. Facklam, and G. M. Carlone. 1997. Limited diversity of Streptococcus pneumoniae psaA among pneumococcal vaccine serotypes. Infect Immun 65:1967-1971.

### Example 1

### Construction of Antigen Expression Vector

General DNA procedures. The plasmids used in the construction of the vectors are listed in Table 1. DNA manipulations were carried out as described in the procedures of Sambrook et al. (43). Transformation of E. coli and Salmonella was done by electroporation (Bio-Rad, Hercules, Calif.). Transformants containing Asd⁺ plasmids were selected on L agar plates without diaminopimelic acid (“DAP”). Only clones containing the recombinant plasmids were able to grow under these conditions. Transfer of recombinant suicide plasmids to Salmonella was accomplished by conjugation using E. coli MGN-617 (Asd⁻) (42) as the plasmid donor. Bacteriophage P22HT int-mediated general transduction was performed by standard methods (50). PCR amplification was employed to obtain DNA fragments for cloning and for verification of chromosomal deletion mutations. The PCR conditions were as follows: denaturation at 95° C. for 20 sec; primer annealing at 55° C. for 20 sec; polymerization at 72° C. for 2 min; and a final extension at 72° C. for 10 min. Nucleotide sequencing reactions were performed using ABI prism fluorescent Big Dye Terminators according to the manufacturer's instructions (PE Biosystems, Norwalk, Conn.).

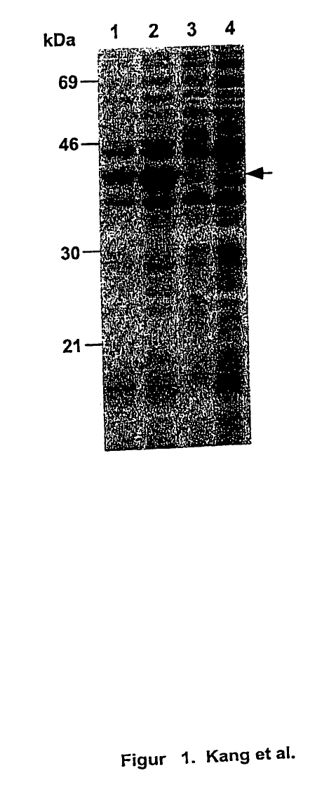

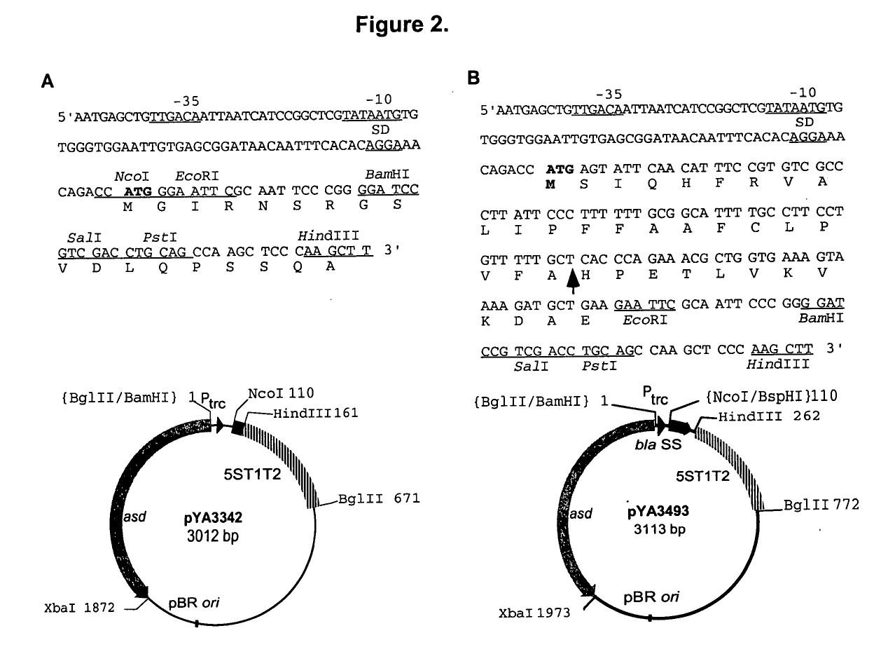

Construction of Asd⁺ vectors to use in the antigen expression. The construction of Asd⁺ vectors is also described in PCT/US01/13915, which is herein incorporated by reference. Carrier bacteria strains harboring mulicopy Asd⁺ vectors (pBRori or pUCori) containing the entire asd gene with its promoter synthesized the Asd protein at a much higher level than necessary to complement the chromosomal asd mutation in a balanced-lethal host-vector system. In fact, the 200 to 300 fold excess production of Asd in a strain such as ×8554 (ΔasdAl6) with the pYA3334 Asd⁺ vector (pUCori) increases the generation time slightly and the LD₅₀ ten-fold compared to the same strain with an Asd⁺ vector with the pSC10lori or pl5Aori. In an attempt to reduce the level of Asd, the asd promoter region was deleted to determine whether there would be sufficient transcription to permit a promoterless asd gene to complement the chromosomal ΔasdA16 mutation. The asd gene sequence was amplified by PCR starting at base pair 286 and ending on base pair 1421 of the S. typhimurium asd sequence (GenBank accession number AF015781) with an N-terminal BglII site and a C-terminal XbaI site. This sequence contains the Shine-Dalgarno (SD) sequence for ribosome recognition but lacks the -35 and -10 promoter sequence and ends just after the asd gene TAG stop codon. The Bgll-XbaI DNA fragment was used to construct Asd⁺ vectors pYA3342 (pBR on) and pYA3341 (pUC on) (Table 1). It was possible to clone this fragment onto pSC10lori or pl5Aori vectors but this did not result in sufficient Asd to permit construction of a balanced-lethal host-vector system with strains such as ×8554 which could grow in the absence of DAP. Both pYA3342 and pYA3341 complemented the asd mutations of E. coli ×6212 and S. typhimurium ×4550. Salmonella strains possessing pYA3342 and pYA3341 produced significantly reduced amounts of Asd protein (39 kDa) compared to strains containing plasmids that had asd genes with the asd native promoter (FIG. 1). pYA3342 and pYA3341 in a Δasd S. typhimurium strain such as ×8554 yielded recombinants that had wild-type LD₅₀s following oral inoculation of BALB/c mice. Plasmid pYA3342 was used for further construction (FIG. 2A).

For the translocation of foreign antigen into the periplasmic space of Salmonella, a recombinant plasmid was constructed by cloning a DNA fragment specifying the signal sequence of β-lactamase. A 105 bp DNA fragment (nucleotides 4049 to 4153 of accession number J01749) of the β-lactamase gene was PCR-amplified from the pBR322 DNA template using a pair of primers, ([N-terminal], 5′GCATTCATGAGTATTCAACATTTCC3′ [SEQ ID NO:4] and ([C-terminal], 5′CCGGAATTCTTCAGCATCTTTTACT3′ [SEQ ID NO:5]). The PCR-amplified fragment included the N-terminus of β-lactamase from the ATG start codon through the signal sequence (23 amino acids) plus 12 amino acids of the N-terminus of the mature β-lactamase (SEQ ID NO:2). These additional 12 amino acid residues were included to increase the efficiency of secretion of the recombinant protein (51). The 105 bp PCR product was digested with BspH1 and EcoRI enzymes and cloned into the Ncol (compatible with the BspHI site) and EcoRI sites of the Asd⁺ vector pYA3342, resulting in plasmid pYA3493 (FIG. 2B). The in-frame position of the β-lactamase signal sequence was confirmed by nucleotide sequencing. Transcription promoted by P_{trc} can be stopped by the 5STIT2 transcriptional terminator located following the multi cloning sites. pYA3493 was stably maintained for 50 or more generation in E. coli ×6212 and S. typhimurium (Δasd) hosts grown in the presence or absence of DAP.

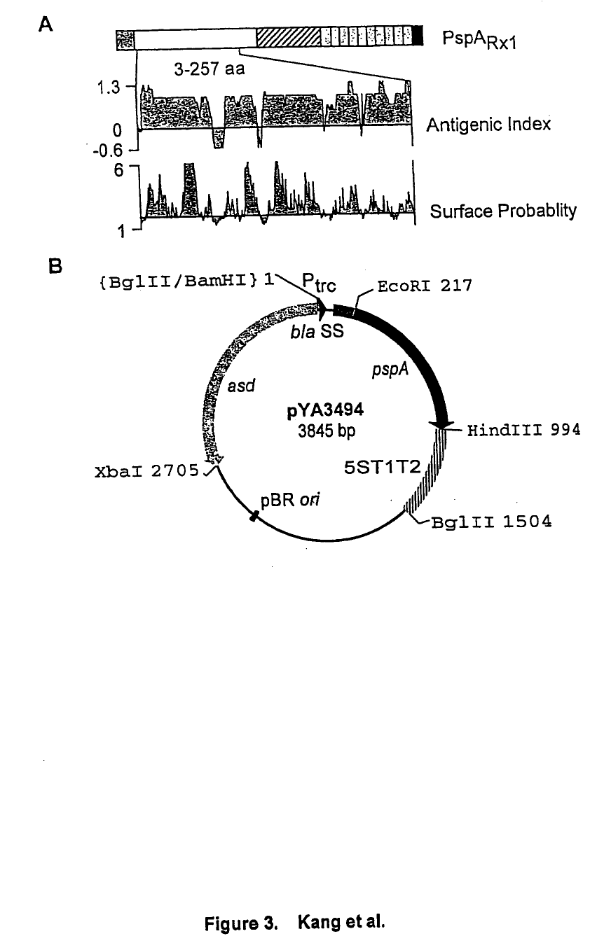

Construction of the rPspA-expressing plasmid. A highly immunogenic α-helical region of PspA from amino acid residues 3 to 257 (765 bp; 255 amino acids) of the mature PspA_{Rx1} protein (588 amino acids) was selected to use as a test antigen in antigen delivery by a Salmonella carrier. The 765 bp DNA fragment of the pspA gene of S. pneumonia Rx1 was PCR-amplified from the pYA3193 DNA template with a pair of primers ([N-terminal], 5′CCGGMTTCTCTCCCGTAGC-CAGTCAGTCT3′ [SEQ ID NO:8], and the same C-terminal primer used in the construction of histidine [6×]-tagged PspA which introduces the TAA TAG stop codons after the pspA coding sequences [SEQ ID NO:7]). The PCR product, digested with EcoRI and HindIII enzymes, was cloned into EcoRI and HindIII sites of pYA3493, resulting in pYA3494 (FIG. 3). The in-frame fusion of the rPspA with the β-lactamase signal sequence was confirmed by nucleotide sequencing. E. coli ×6212 harboring pYA3494 expressed rPspA as approximately 1% of the total cell protein.

Purification of recombinant PspA. For overexpression of histidine (6×)-tagged PspA, a fragment of the pspA gene of S. pneumoniae Rx1 was PCR-amplified from pYA3193 (36) template DNA using a pair of primers ([N-terminal] 5′CCGGAATTCATCACCATCACCATCACTCTCCC-GTAGCCAGTCAGT3′ [SEQ ID NO:6], and [C-terminal] 5′GGGAAGCTTCTATTATTCTACA-TTATTGTT3′ [SEQ ID NO:7]). The 0.8 kb amplified fragment was then cloned into the pYA3342 vector, resulting in pYA3496 (Table 1). The N-terminal primer contains an EcoRI site and six consecutive histidine codons (alternate use of CAT and CAC) for histidine (6×) tagging at the N-terminus. The C-terminal primer specifies two consecutive stop codons (TAA TAG) followed by a HindIII site. In-frame cloning was confirmed by nucleotide sequencing. E coli ×6212 harboring pYA3496 expressed a large amount of soluble histidine (6×)-tagged rPspA in its cytoplasmic fraction. According to manufacturer's protocol (Qiagen, Valencia, Calif.), rPspA protein was purified by an affinity purification process with Ni²⁺-nitrilotriacetic acid-agarose support. The protein purity was verified by Coomassie blue staining of SDS-PAGE gels, and the total amount of purified protein was determined by using the Pierce protein assay kit (Pierce, Rockford, Ill.) with BSA as a standard. An immunoblot with the Xi126 PspA monoclonal antibody (31) was performed to confirm the purified protein.

### Example 2

### Construction of Carrier Bacteria

Bacterial strains, media and growth conditions. Bacterial strains are listed in Table 1. Bacteriophage P22HTint (45) was used for generalized transduction. Escherichia coli and S. typhimurium cultures were grown at 37° C. in Lennox broth (28) or Luria-Bertani (LB) broth, or on LB agar (1). MacConkey agar (Difco, Detroit, Mich.) supplemented with 1% sugar was used for fermentation assays. The utility of Asd⁺ plasmids in bacterial live vaccines is described elsewhere (35). When required, antibiotics were added to culture media at the following concentrations: ampicillin, 100 μg/ml; chloramphenicol, 30 μg/ml; kanamycin, 50 μg/ml; tetracycline, 15 μg/ml. Diaminopimelic acid (DAP) was added (50 μg/ml) for the growth of Asd⁻ strains (35). LB agar containing 5% sucrose was used for sacB gene-based counter selection in the allelic exchange experiments (17). S. pneumoniae WU2 was cultured on Brain heart infusion (BHI) agar containing 5% sheep blood or in Todd-Hewitt broth plus 0.5% yeast extract (THY) (6).

Construction of a S. typhimurium vaccine strain. The Δcrp mutation was introduced into S. typhimurium ×3339 by allelic exchange using the suicide vector pMEG-493 to yield ×8499. The presence of the 680 bp deletion was confirmed by PCR with a primer set flanking crp (5′-AAAG-TCGCAATGGMGGC-3′ [SEQ ID NO:9] and 5′-CGTAGACGACGATGGTCTTG-3′ [SEQ ID NO:10]) and a strain phenotype of Mal⁻ and non motility. The ΔasdA16 mutation was then introduced into ×8499 using P22HTint transduction from ×8554 with the suicide vector pMEG-443 integrated into a strain with the ΔasdA16 mutation followed by sucrose selection to eliminate the suicide vector to yield ×8501 (26). The presence of the 1,242 bp asd deletion in ×8501 was confirmed by PCR using flanking asd primer set

| (5′-CGGAAATGATTCCCTTCCTAACG-3′ | [SEQ ID NO:11] |
|---|---|
| and | |
| 5′-TATCTGCGTCGTCCTACCTTCAG-3′ | [SEQ ID NO:12]) |
| (26). | |

Characterization of phenotype. MacConkey agar supplemented with 1% maltose was used to detect the phenotype of Salmonella crp mutants. Motility was evaluated by observing Salmonella spread on a semisolid medium composed of 1% casein enzyme hydrolysate, 0.5% NaCl, and 0.5% agar. Triphenyltetrazolium chloride (50 μg/ml) was added to motility medium to observe Salmonella as red-colored cells. The presence of the ΔasdA16 mutation in Salmonella was confirmed by inability of the strain to grow on media without DAP (35). Lipopolysaccharide profiles of Salmonella strains were examined by described methods (23).

### Example 3

### Expression and Secretion of Antigen

Expression and subcellular localization of rPspA in Salmonella. A S. typhimurium strain was constructed to examine expression and subcellular localization of rPspA. The atrB13::MudJ allele (14), causing constitutive expression of β-galactosidase, in S. typhimurium JF2430 was transduced into S. typhimurium ×8554 by P22HT int-mediated generalized transduction (50), resulting in ×8599 (hisG ΔasdA16 atrB13::MudJ). ×8599 was Lac⁺ on MacConkey agar plus lactose and DAP. β-galactosidase production from the atrB13::MudJ allele in ×8599 was used as a cytoplasmic protein marker and as an indicator of membrane leaking in the examination of subcellular fractionations. To observe rPspA expression, plasmid pYA3494 was introduced into S. typhimurium ×8599. ×8599 harboring pYA3493 (vector alone) was used as the control.

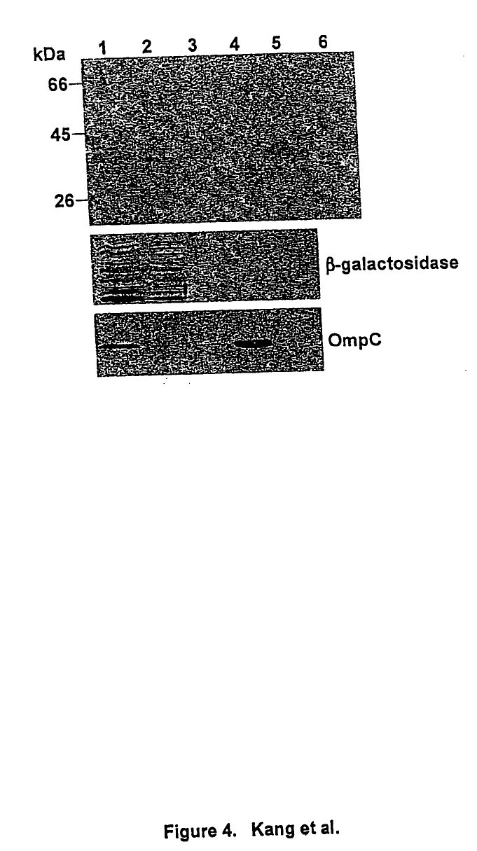

With the expectation of the periplasmic secretion of the rPspA, various subcellular fractions including cytoplasm, periplasm, outer membrane, and culture supernatant of ×8599 (pYA3494) were prepared to examine the location of rPspA. Although the calculated size of rPspA was approximately 30 kDa, PspA-specific monoclonal antibody Xi126 reacted with an approximately 35 kDa protein (FIG. 4). Aberrant migration of a PspA protein has been seen in previous studies (36, 52). Although a large amount of the rPspA resided in the cytoplasmic fraction, half of the rPspA was detected in the periplasmic fraction and the culture supernatant fluid. Little or no rPspA was detected in the outer membrane fraction. Densitometry analyses of immuno reactive bands showed that approximately 50% of the rPspA was located in both periplasm (25%) and culture supernatants (25%). In the immunoblot analyses of subcellular fractions with anti-β-galactosidase and -OmpC monoclonal antibodies, the β-galactosidase and OmpC proteins were detected in the cytoplasm and outer membrane fractions, respectively, suggesting that the rPspA detected in the periplasmic fraction and culture supernatant fluid was actively secreted instead of resulting from non-specific membrane leaking or cell death.

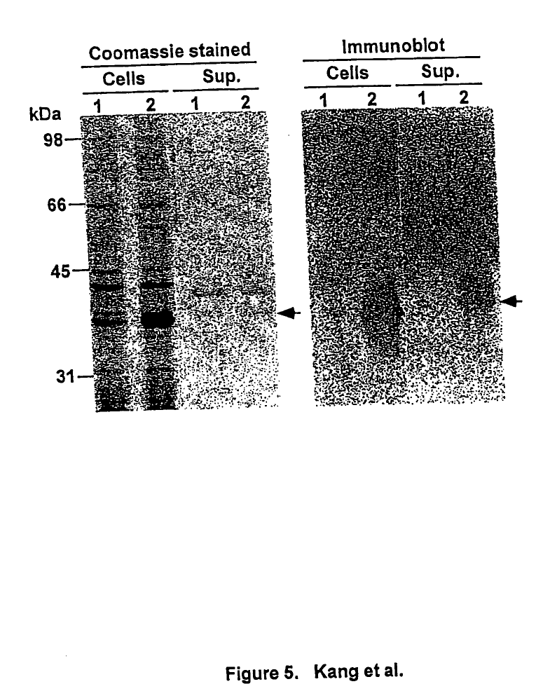

Recombinant S. typhimurium Δcrp-28 vaccine expressing rPspA antigen. pYA3493 (vector control) and pYA3494 encoding rPspA were electroporated into the Δcrp-28 ΔasdA16 strain ×8501. S. typhimurium ×8501 (Δcrp-28 ΔasdA16) vaccine strain containing pYA3494 expressed the rPspA protein at an approximate molecular mass of 35 kDa. In the analyses of Coomassie blue-stained SDS-PAGE, the amount of rPspA protein was as much as approximately 1-2% of total proteins of ×8501 (pYA3494) strain (FIG. 5). With results consistent with those seen in the rPspA localization analysis [75% of rPspA cell-associated (50% cytoplasm and 25% periplasm) and 25% of rPspA secreted], the rPspA expressed in the ×8501 vaccine strain was secreted into the culture supernatant along with other secreted proteins. To examine the stability of plasmids pYA3493 and pYA3494 in Salmonella ×8501 in vitro, ×8501 cells containing pYA3493 and pYA3494 were cultured with daily passage for five consecutive days in L broth containing DAP. All ×8501 clones examined (300 clones/day) kept the Asd⁺ plasmid pYA3493 and pYA3494, indicating that pYA3493 and pYA3494 were very stable in the ×8501 vaccine strain. Cells obtained from the last day culture of the stability test expressed similar amounts of the 35 kDa rPspA compared to those from the first day (data not shown), suggesting stable expression of rPspA without rearrangements.

Methods

SDS-PAGE and immunoblot analyses. Protein samples were boiled for 5 min and then separated by discontinuous SDS-polyacrylamide gel electrophoresis (SDS-PAGE). Protein bands were visualized by Coomassie brilliant blue R250 (Sigma, St. Louis, Mo.) staining. For immunoblotting, proteins separated by SDS-PAGE were transferred eletrophoretically to nitrocellulose membranes. The membranes were blocked with 3% bovine serum albumin in 10 mM Tris-0.9% NaCl (pH 7.4) and incubated with mouse monoclonal antibodies specific for PspA (Xi126) (31), OmpC (48), or, β-galactosidase (Sigma), and then with a peroxidase-conjugated goat anti-mouse immunoglobulin G (Bio-Rad). Immuno-reactive bands were detected by the addition of 4-chloro-1-naphthol (Sigma) in the presence of H₂O₂. The reaction was stopped after two minutes by washing with several large volumes of deionized water.

Salmonella subcellular fractionation. The periplasmic fraction was prepared by a modification of the lysozyme-osmotic shock method (54). Cultures grown in LB broth to 0.8 OD₆₀₀ were centrifuged at 7,000×g for 10 min and the supernatant fluid saved for analysis of secreted proteins. The cell pellets were resuspended in 800 μl of 100 mM Tris-HCl buffer (pH 8.6) containing 500 mM sucrose and 0.5 mM EDTA. Hen egg-white lysozyme (40 μl of 4 mg/ml stock solution) was added, followed immediately by the addition of 3.2 ml of 50 mM Tris-HCl buffer (pH 8.6) containing 250 mM sucrose 0.25 mM EDTA, and 2.5 mM M⁹Cl₂. After gentle agitation, the suspension was incubated for 15 min in an ice bath. Cells were removed by centrifugation at 7,000×g for 6 min followed by filtration of the supernatant through a 0.45 μm filter. The filtered supernatant fluid served as the periplasmic fraction. Cells resuspended in 4 ml of 20 mM Tris-HCl (pH 8.6) were disrupted by two passages through a French pressure cell (American Instrument Company, Silver Spring, Md.). Cell lysates were centrifuged at 7,000×g at 4° C. for 6 min to remove unbroken cells. The supernatant fluid was then centrifuged at 132,000×g at 4° C. for 1 h to separate the soluble fraction and insoluble cell envelopes. The soluble fraction served as the cytoplasmic proteins. To isolate the outer membrane fraction, total envelope pellets were suspended in 4 ml of 20 mM Tris-HCl (pH 8.6) containing 1% sarkosyl and incubated for 30 min on ice. The outer membrane fraction was obtained as a pellet after centrifugation at 132,000×g at 4° C. for 1 h. The pellet was resuspended in 4 ml of mM Tris-HCl buffer (pH 8-6). The original culture supernatant was filtered (0.22 μm filter) and secreted proteins were precipitated with 10% trichloroacetic acid (1 h, 4° C.). An equivalent amount of each fraction sample was separated by SDS-PAGE for western blot analysis. Using the outer membrane protein preparation procedure described above, Salmonella outer membrane proteins (SOMPs) were prepared from S. typhimurium ×4746 cells grown in LB broth without galactose for analysis by ELISA. SOMPs obtained from ×4746 preclude LPS O-antigen contamination.

### Example 4

### Characterization of Immune Response and Demonstration of Protective Immunity

Immunization of mice. Two groups of 5 inbred 7-week old female BALB/c mice were deprived of food and water for 4 h before infection. The recombinant S. typhimurium ×8501 (pYA3494) vaccine (1.9×1 09 CFU in 20 μl of phosphate-buffered saline containing 1% gelatin [BSG]) grown in LB broth to 0.8 OD₆₀₀ was orally administered to BALB/c mice. The recombinant S. typhimurium ×8501 (pYA3493) vaccine (2×10⁹ CFU in 20 μl of BSG) was used as a vector control. Food and water were returned to the immunized mice 30 min after immunization. Blood was obtained by retro-orbital puncture with heparinized capillary tubes at biweekly intervals. Following centrifugation at 4,000×g for 5 min, the serum was removed from the whole blood and stored at −20° C. Vaginal secretion specimens were collected in a 50 μl BSG wash and stored at −20° C. (57).

Pneumococcal challenge. To observe the immune responsiveness of pneumococcal infection after vaccination, a sublethal dose of 3.8×10⁵ S. pneumoniae WU2 CFU in 200 μl of BSG was administered by intravenous (i.v.) injection to S. typhimurium vaccine-immunized BALB/c mice at sixteen weeks after primary immunization. The ability of the Salmonella-PspA vaccine to protect the immunized mice against S. pneumoniae was assessed by intraperitoneal (i.p.) challenge with 4.8×10³ CFU of S. pneumoniae in 100 μl of BSG. The LD₅₀ of S. pneumoniae WU2 in BALB/c mice was >10⁶ by i.v. and <10² by i.p.

ELISA. ELISA was used to assay antibodies in vaginal secretions and serum to S. typhimurium LPS and SOMPs, and rPspA. Polystyrene 96-well flat-bottom microtiter plates (Dynatech Laboratories Inc., Chantilly, Va.) were coated with S. typhimurium LPS (100 ng/well, Sigma), SOMPs (100 ng/well), or purified rPspA (100 ng/well). Antigens suspended in sodium carbonate-bicarbonate coating buffer (pH 9.6) were coated with 100 μl volumes in each well. The coated plates were incubated at 37° C. for 1 h followed by an overnight incubation at 4° C. Free binding sites were blocked with a blocking buffer (PBS [pH 7.4]-0.1% Tween 20 and 1% bovine serum albumin). Vaginal secretions and sera obtained from the same experimental group (5 mice/group) were pooled and diluted 1:10 and 1:600, respectively. A 100 μl volume of diluted samples was added to individual wells in duplicate and incubated for 2 h at 37° C. Plates were treated with biotinylated goat anti-mouse IgG, IgG1 or IgG2a (Southern Biotechnology Inc., Birmingham, Ala.) for sera and IgA for vaginal secretions. Wells were developed with streptavidin-alkaline phosphatase conjugate (Southern Biotechnology) followed by p-nitrophenylphosphate substrate (Sigma) in diethanolamine buffer (pH 9.8). Color development (optical density) was recorded at 405 nm using an automated ELISA plate (model EL31 1SX; Biotek, Winooski, Vt.). Absorbance readings two times higher than pre-immune serum baselines were considered positive reactions.

Immune responses in mice by oral immunization with the recombiuant S. typhimurium vaccines. All mice orally administered with a single dose of 1.3×10⁹ CFU S. typhimurium ×8501 (pYA3493, vector control) survived for the 30 day monitoring period. Those mice were protected against wild-type ×3339 (LD₅₀<10⁶) challenge (1.7×10⁹ CFU) 30 days after initial administration. There were no survivors in a group of unimmunized mice challenged with 1.7×10⁷ CFU of ×3339. These results indicate that ×8501 with an Asd⁺ vector is avirulent for mice and elicits a protective immune response against S. typhimurium challenge.

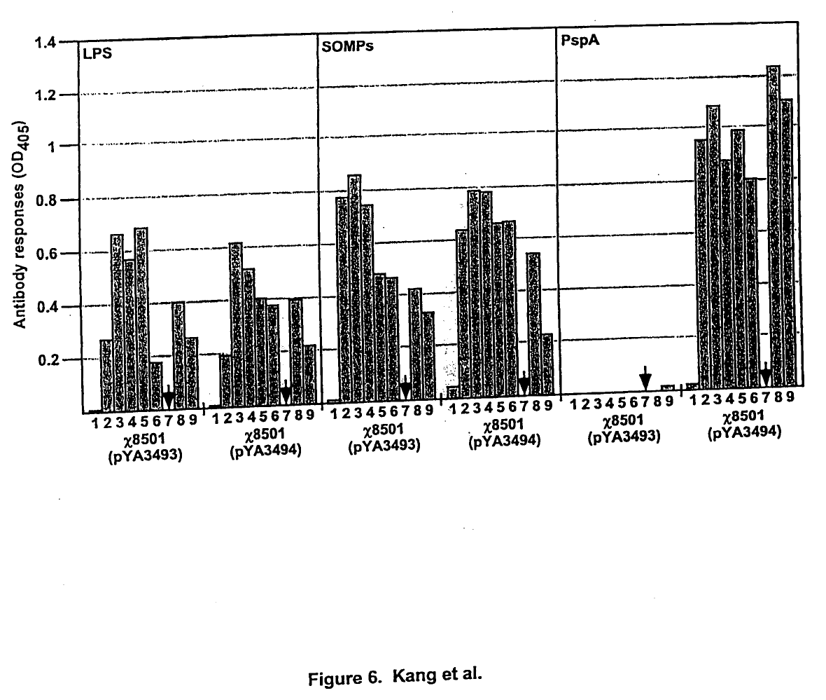

A single dose of S. typhimurium ×8501 (pYA3494) (1.9×10⁹ CFU) or ×8501 (pYA3493) (control, 2×10⁹ CFU) was orally administered to 7 week-old female BALB/c mice. All immunized mice survived and no signs of disease in the immunized mice during the entire experimental period were observed. The antibody responses to Salmonella LPS and outer membrane proteins (SOMPs), and to the foreign antigen rPspA were measured from the sera and the vaginal secretions of the immunized mice. The serum IgG responses to LPS, SOMPs and rPspA are presented in FIG. 6. At two weeks after administration, little IgG responses to the antigens were observed. Maximal anti-LPS, -SOMPs and -rPspA IgG levels without boost immunization were detected at 6 weeks post immunization, Anti-LPS IgG levels were lower than IgG specific for SOMPs and rPspA.

At 17 weeks post immunization, mice were infected intravenously with a sublethal dose (3.8×10⁵ CFU) of the virulent S. pneumoniae WU2 strain to monitor the changes of anti-rPspA antibody titers. Sublethal i.v. infection with S. pneumoniae did not kill mice immunized with ×8501 (pYA3493) and ×8501 (pYA3494) vaccines. Because native PspA is a highly immunogenic pneumococcal surface protein, the pneumococcal challenge induced and boosted rPspA specific immune responses in ×8501 (pYA3493) and ×8501 (pYA3494) immunized mice, respectively (FIG. 6). In comparison to the anti-rPspA IgG level (OD₄₀₅ 0.81) at 12 weeks post S. typhimurium ×8501 (pYA3494) immunization, the pneumococcal challenge boosted 53% more anti-rPspA IgG (OD₄₀₅ 1.24) one week later. This suggests that S. typhimurium ×8501-rPspA vaccine induces immunological memory for a rapid responsiveness to subsequently administered PspA antigen. Importantly, the anti-rPspA IgG levels were higher than those to Salmonella LPS and SOMPs. A comparison of maximum IgG levels induced to each antigen by the ×8501 (pYA3494) vaccine reveals that the anti-rPspA IgG level (OD₄₀₅ 1.1) was 36% higher than for LPS (OD₄₀₅ 0.7) and 27% higher than for SOMPs (OD₄₀₅ 0.8). In another experiment in which BALB/c mice were orally immunized with ×8501 (pYA3494), the anti-PspA and anti-LPS IgG titers were A₄₀₅=2.4 and A₄₀₅=1.65, respectively, 8 weeks after immunization; A₄₀₅=2.65 and A₄₀₅=1.33, respectively, 3 weeks after a boost at 16 weeks after primary immunization. Anti-rPspA IgG was not detected in sera obtained from mice immunized with ×8501 (pYA3493), the vector control vaccine. The ×8501 (pYA3493) vaccine elicited anti-LPS and -SOMPs IgG responses with similar kinetics and levels to those induced by ×8501 (pYA3494). These results suggest that Salmonella-delivered rPspA antigen had minimal influence on the immune response to Salmonella itself.

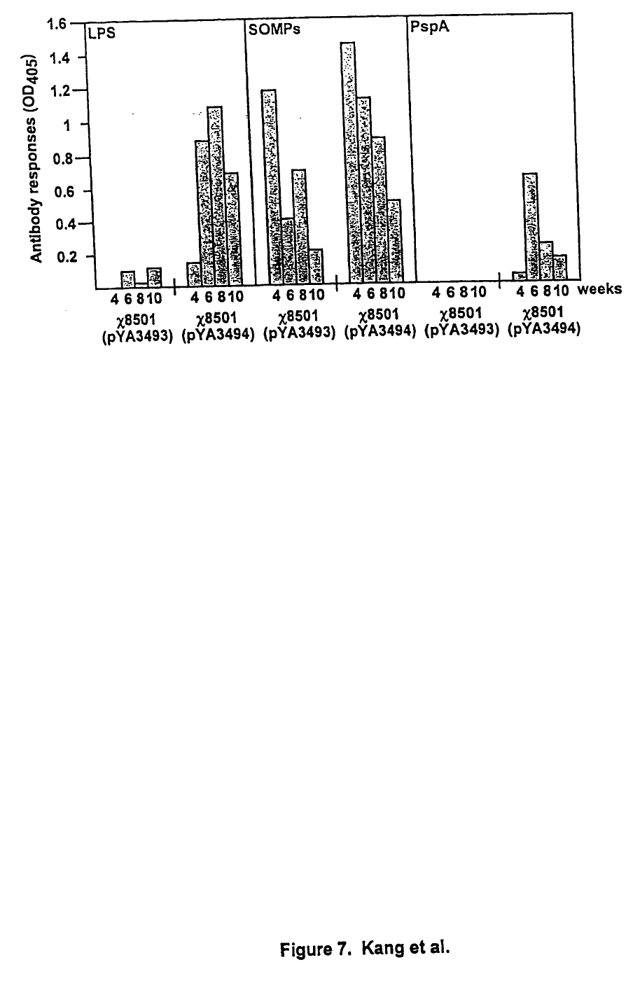

IgA levels, mostly secretory IgA, for LPS, SOMPs and rPspA were measured from the vaginal fluids of immunized mice. Vaccines ×8501 (pYA3493) and ×8501 (pYA3494) elicited anti-LPS and anti-SOMPs IgA. rPspA specific IgA was detected in the vaginal fluids from mice immunized with ×8501 (pYA3494) but not ×8501 (pYA3493) vector-only control (FIG. 7).

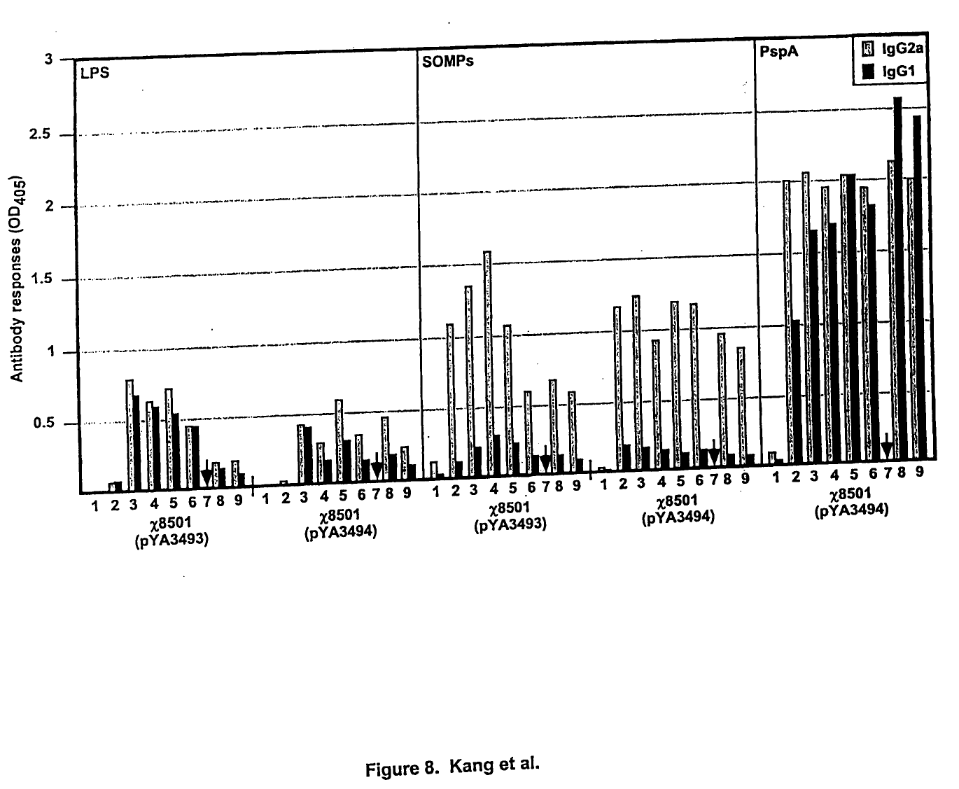

IgG isotype analyses. The type of immune responses to Salmonella LPS and SOMPs and the rPspA was further examined by measuring the levels of IgG isotype subclasses IgG2a and IgG1. The ThI-helper cells direct cell-mediated immunity and promote class switching to IgG2a, and Th2 cells provide potent ‘help’ for B cell antibody production and promote class switching to IgG1 (38, 49). IgG2a isotype dominant responses were observed for the Salmonella LPS and SOMPs antigens (FIG. 8). The ratios of IgG2a/IgG1 for anti-LPS and -SOMPs in sera obtained from mice immunized with X8501 (pYA3493) were similar to those observed in mice immunized with ×8501 (pYA3494). The ratios of IgG2a/IgG1 for anti-SOMPs (ranging from 6.4 to 11.5) are higher than that for LPS (ranging from 1.1 to 2.5). Th1-type dominant immune responses are frequently observed after attenuated Salmonella immunization (29, 39). In contrast to the type of immune responses to LPS and SOMPs, Thl- and Th2-type mixed responses were observed for the rPspA antigen. Although the IgG2a levels were higher than IgG1 levels in the early phase (up to 8 weeks post immunization), the level of anti-rPspA IgG1 isotype antibodies gradually increased. After 10 weeks post immunization, a 1:1 ratio of IgG2a to IgG1 or IgG1 dominant responses were detected (FIG. 8). The pneumococcal i.v. challenge maintained the IgG1 dominant immune responses to PspA as seen before challenge.

Evaluation of protective immunity. To examine the ability of Salmonella-rPspA vaccines to protect against pneumococcal infection, BALB/c mice were immunized with either S. typhimurium ×8501 (pYA3493) (1.3×10⁹ CFU dose) or ×8501 (pYA3494) (1.7×10⁹ CFU dose). Ten weeks after initial immunization, a second 10⁹ CFU dose of each vaccine was administered. We did not detect weakness or disease signs in vaccinated mice during the immunization periods. At 5 weeks after the second immunization, mice were challenged intraperitoneally with 4.8×10³ CFU of S. pneumoniae WU2. Sixty percent of the mice immunized with ×8501 (pYA3494) were protected from pneumococcal challenge. This challenge dose killed 100% of unimmunized and ×8501 (pYA3493) immunized mice (Table 2). Following challenge mice unimmunized or immunized with ×8501 (pYA3493) died much quicker with a mean day of death of 2 compared to mice immunized with ×8501 (pYA3494) which had a mean day of death of 5.

TABLE 2
Oral immunization of rPspA-expressing S. typhimurium χ8501 (pYA3494) vaccine
protects BALB/c mice against challenge with virulent S. pneumoniae WU2
strain

| | RPspA | Protection^{c} | | |
|---|---|---|---|---|
| Vaccines^{a} | Expression^{b} | (% alive) | Days to death | P value^{d} |
| χ8501 (pYA3494) | + | 60 | 5, 5 | |
| | | | >21, >21, >21 | |
| χ8501 (pYA3493) | − | 0 | 1,2,2,3,3 | 0.0137 |
| Unimmunized | N/A | 0 | 1,2,2,2,3 | 0.0125 |

^{a}Mice were orally immunized a total of two times at 16 weeks intervals with ˜10⁹ CFU dose of indicated vaccine strains.
^{b}+, rPspA expressed; −, rPspA not expressed; N/A, not applicable.
^{c}Five weeks after the last immunization, mice (5 mice/group) were challenged intraperitoneally (i.p.) with approximately 4.8 × 10³ CFU of virulent S. pneumoniae WU2. The LD⁵⁰ of WU2 by i.p. route infection in unimmunized BALB/c mice was <10² (data not shown). Mortality was monitored for three weeks after pneumococcal challenge.
^{d}P values comparing the days to death were calculated by Student's t test.

### Example 5

### Construction of Plasmid pYA3605, Comprising PspA_{EF5668}/PspA_{Rx1} Fusion

The vast majority of S. pneumoniae serotypes can be grouped into two families (Family 1 and Family 2) with regard to the presence of PspA antigens that share significant immunological relationships as determined by the use of a large bank of monoclonal antibodies against various PspA proteins and the analysis of PspA proteins from a great diversity of S. pneumoniae strains of diverse capsular polysaccharide serotypes. FIG. 9 diagrammatically depicts the familial relationship among many of these diverse serotypes. PspA proteins from Family 1 are not closely related to PspA proteins from Family 2. To construct pYA3605, which comprises polynucleotides encoding fragments of PspA from both Family 1 and Family 2 S. pneumoniae strains, S. pneumoniae strain EF5668 was selected to represent Family 2 and S. pneumoniae strain Rx1 wa selected to represent Family 1. To construct a recombinant attenuated vaccine that would induce an antibody immune response that would be protective against infection with the greatest diversity of S. pneumoniae capsular polysaccharide serotype strains, the coding sequences for the α-helical domains of the PspA antigens from the S. pneumoniae EF5668 and Rx1 strains were fused.

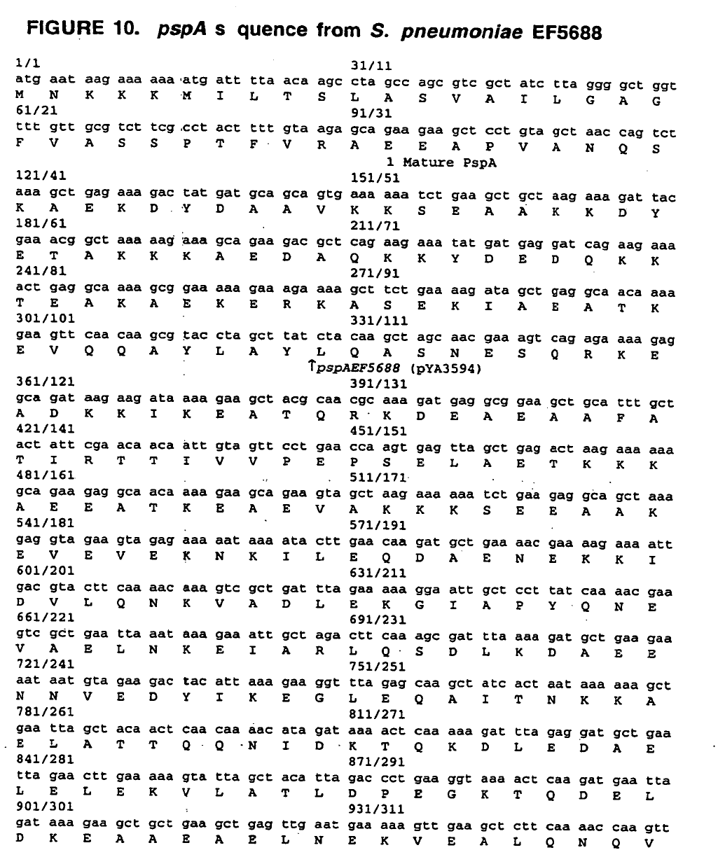

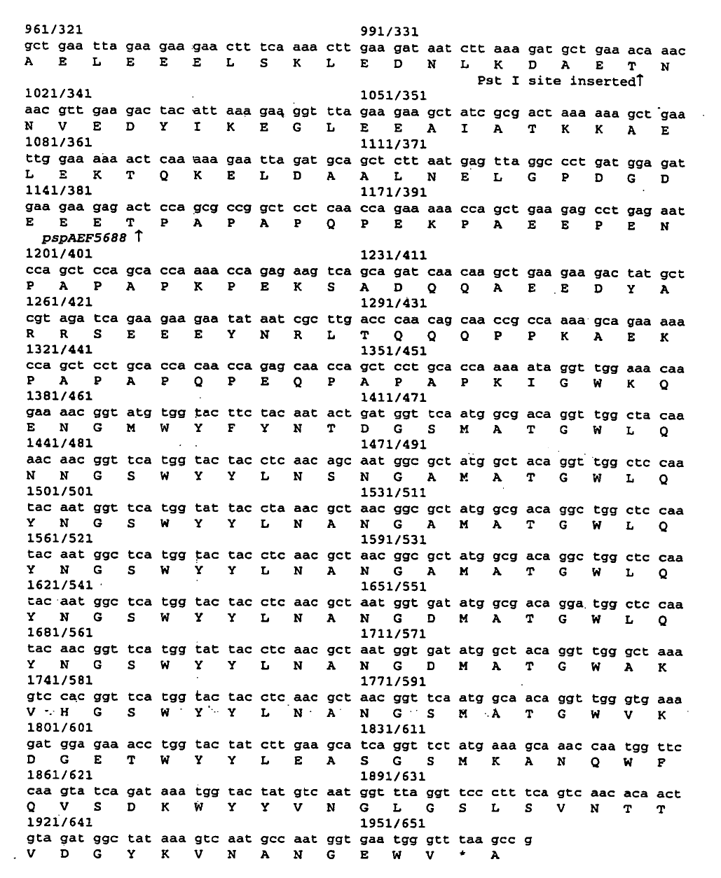

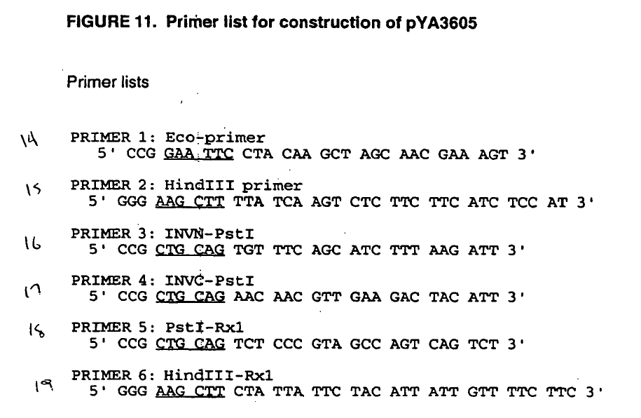

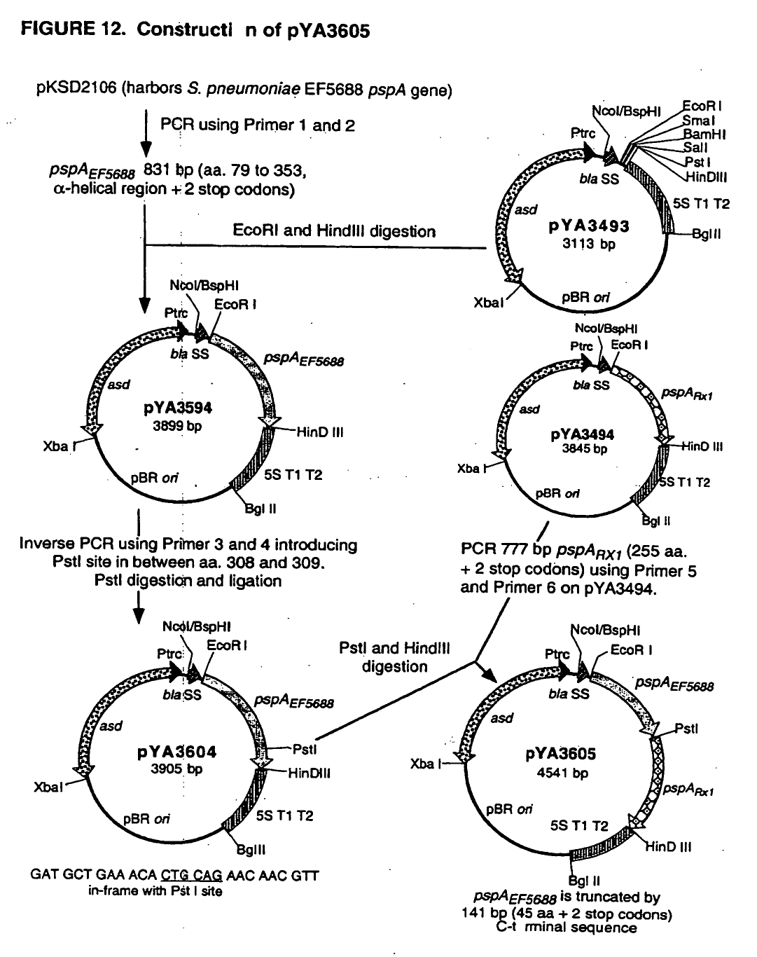

FIG. 10 gives the nucleotide sequence for the N-terminal portion of the EF5668 pspA gene (SEQ ID NO:13 represents the nucleotide sequence and SEQ ID NO:26 represents the translation product sequence), including the signal sequence and the α-helical domain (as depicted in SEQ ID NO:1). FIG. 11 lists all the oligonucleotide primers to first clone the EF5668 α-helical domain into pYA3493 to yield pYA3594 (FIG. 12) and then proceed with the construction of pYA3605 with the EF5668-Rx1 fusion of the α-helical domains of both PspA antigens (FIG. 12). Primers 1 and 2 (SEQ ID NO:14 and SEQ ID NO:15, respectively) (FIG. 11) were used to amplify by PCR an 831 bp sequence of the EF5668 pspA sequence present on pKSD2106 (McDaniel et al., Infect. Immune. 1998. 66:4748-4754), which encodes amino acids 79 to 353 of the mature PspA protein after cleavage of the 31 amino acids in the PspA signal sequence. Primer 2 (SEQ ID NO:15; FIG. 11) also specified the stop codons TGA TM after PspA_{EF5668} amino acid 353. The PCR product was digested with EcoRI and HindIII and ligated to the Asd⁺ bla SS vector pYA3493, which also had been digested with EcoRI and HindIII.

This new plasmid was transformed into ×6212(pYA232) and designated pYA3594 (FIG. 12). To facilitate construction of the fusion PspA protein, Primers 3 and 4 (SEQ ID NO:16 and SEQ ID NO:17, respectively; FIG. 11) were used for inverse PCR with pYA3594 (FIG. 12) to introduce a PstI recognition site between amino acids 308 and 309 in the mature PspA_{EF5668} sequence (SEQ ID NO:26; FIG. 10). The DNA fragment generated by inverse PCR was digested with PstI and ligated to yield pYA3604 (FIG. 12), which was transformed into strain ×6212 (pYA232). DNA primers 5 and 6 (SEQ ID NO:18 and SEQ ID NO:19, respectively) were then used to amplify by PCR a 777 bp DNA sequence from pYA3494 (FIG. 12) encoding amino acids 3 to 257 of PspA_{RX1} and the two stop codons TM TAG. This DNA fragment was digested with PstI and HindIII and ligated with pYA3604, which was also digested with PstI and HindIII, to generate pYA3605 (FIG. 12), which was transformed into ×6212 (pYA232). This construction led to the deletion of 45 amino acids from the Psp_{AEF5668} protein encoded in pYA3594.

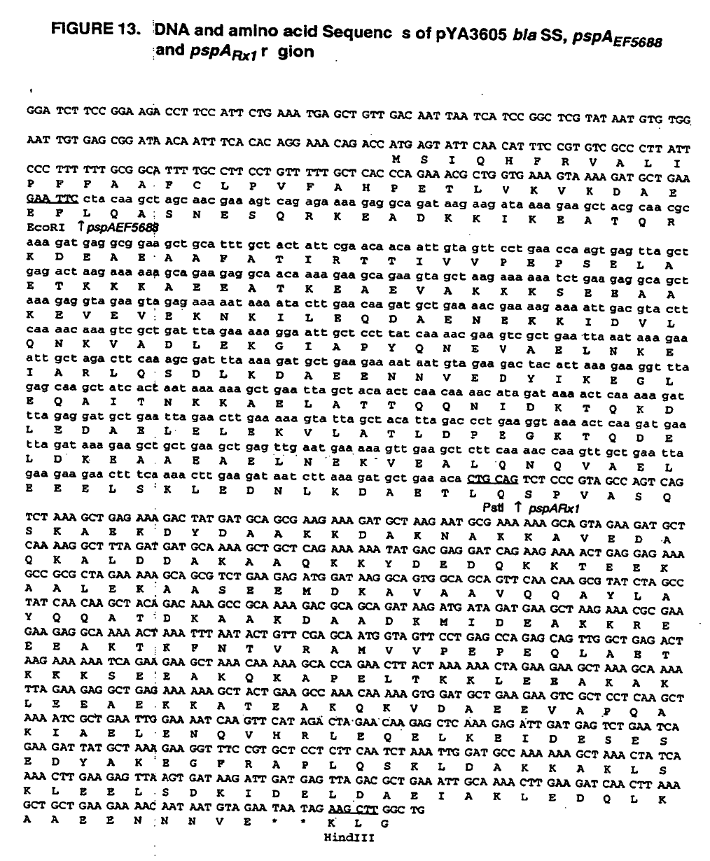

The final construct thus specifies the β-lactamase signal sequence and first 12 amino acids, two codons for the EcoRI site, 230 amino acids of the PspAEF5668 protein (mature amino acids 79 to 308), two codons for the PstI site and 255 amino acids of the PspA_{Rx1} protein (mature amino acids 3 to 257) followed by two stop codons (SEQ ID NO:25; FIG. 13). The entire fusion encoded in pYA3605 has 524 amino acids with 501 remaining after cleavage of the signal sequence.

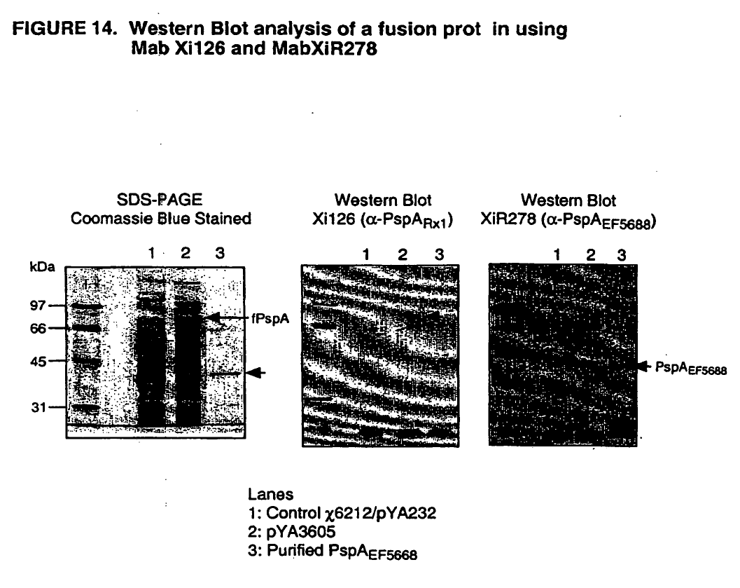

All of the recombinant plasmids described above were transformed into ×6212 (pYA232). pYA232 has a pSC101 replicon that is compatible with the pBR ori present in all the plasmid constructs described above. pYA232 also possesses the lacl^{q} gene so the expression of genes under the control of the P_{trc} promoter is repressed. This requires the addition of the inducer IPTG to relieve Lacl^{q} repression and permit transcription of the PspA encoding genes in all of the recombinant vectors described above. Growth of ×6212 (pYA232, pYA3605) in Luria broth containing 1 mM IPTG and analysis of the proteins synthesized by SDS-PAGE followed by western blot analysis with MAb Xi126 against the Rx1 PspA and MAb XiR278 against the EF5668 PspA (FIG. 14) revealed a fusion protein of the anticipated size and possessing determinants recognized by both monoclonal antibodies.

### Example 6

### Construction of pYA3620 Containing the N-Terminal Signal Sequence and C-Terminal Portions of the β-Lactamase Gene

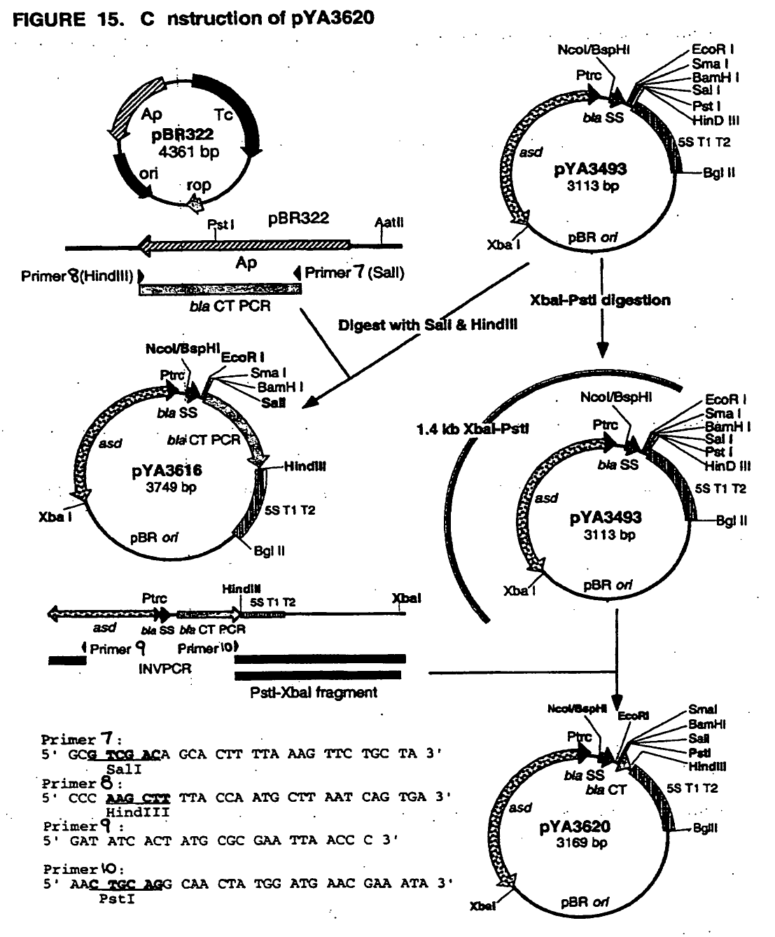

In addition to the 12 amino acids at the N-terminal end of the mature β-lactamase protein being important for β-lactamase secretion, some evidence suggests that the 21 amino acid sequence from the C-terminal end of β-lactamase (SEQ ID NO:3) might also enhance secretion across the cytoplasmic membrane. Therefore the Asd⁺ β-lactamase secretion vector pYA3493 (FIG. 15) has been modified to encode the C-terminal 22 amino acids of the β-lactamase protein.

Oligonucleotide primers 7 and 8 (SEQ ID NO:21 and SEQ ID NO:22, respectively; FIG. 15) were used to amplify by PCR a 660 bp DNA sequence from the β-lactamase gene present in pBR322 (FIG. 15) that specifies ampicillin resistance. This sequence contains the entire C-terminal portion of β-lactamase and because of the restriction enzyme recognition sequences encoded in primers 7 and 8, contains SalI and HindIII restriction sites. This PCR-generated DNA fragment was therefore digested with SalI and HindIII and cloned into pYA3493, which had also been digested with SalI and HindIII. This resulted in the recombinant plasmid pYA3616 (FIG. 15). Using oligonucleotide primers 9 and 10 (SEQ ID NO:23 and SEQ ID NO:24, respectively; FIG. 15) for an inverse PCR reaction with pYA3616 yielded a ˜2.0 kb DNA fragment, which deleted 596 bp of the C-terminal portion of the bla gene initially amplified by PCR, as described above. This DNA fragment was digested with PstI and XbaI to yield a ˜1.8 kb DNA fragment that was ligated to a ˜1.4 kb DNA fragment generated from pYA3493 by PstI and XbaI digestion. The ligated plasmid, which is designated pYA3620 (FIG. 15), was transformed into ×6212. pYA3620 possesses the β-lactamase signal sequence and N-terminal 12 amino acid sequence present in pYA3493 and a 22 amino acid C-terminal amino acid sequence of β-lactamase following a PstI restriction site in pYA3620.

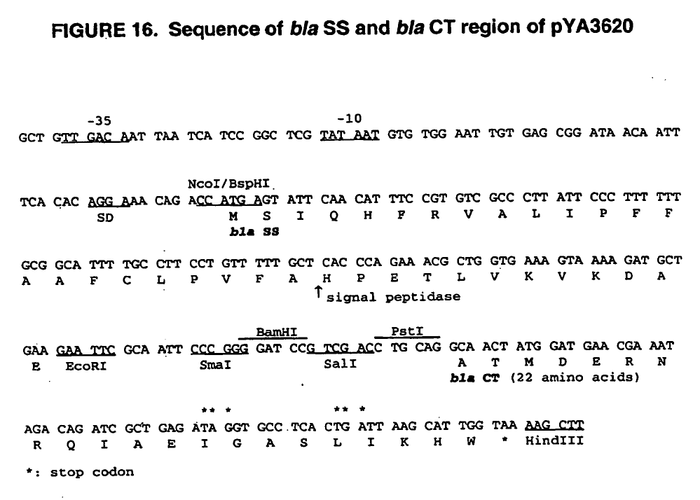

As constructed, pYA3620 will lead to the synthesis of the β-lactamase signal sequence followed by codons for the first 12 amino acids in the secreted form of β-lactamase and then 24 amino acids encoded in the multiple cloning site and the beginning of the out-of-frame coding sequence for the C-terminal β-lactamase sequence. The TAG stop codon for this peptide is identified by *** in FIG. 16 (SEQ ID NO:25) which presents the nucleotide sequence encoding P_{trc}, the β-lactamase signal sequence plus N-terminal β-lactamase 12 amino acids, the multiple restriction enzyme recognition sequences as multiple cloning sites and the β-lactamase C-terminal 21 amino acid sequence present in pYA3620.

Although the β-lactamase C-terminal sequence is out of frame, the cloning of any sequence encoding a protective antigen can be generated by PCR with N-terminal EcoRI and C-terminal PstI cleavage sites such that an in-frame fusion of the N-terminal β-lactamase sequence to the protective antigen to the C-terminal β-lactamase sequence will result. Insertion of the pspA encoding sequence from pYA3494 into pYA3620 can be achieved by PCR amplification of the pspA DNA fragment in pYA3494 using oligonucleotide primer 5′ CCG GAA TTC TCT CCC GTA GCC AGT CAG TCT (SEQ ID NO:8) to encode an EcoRI site and the N-terminal PspA amino acids 3 to 9 and the primer 5′ AAC TGC AG TTC TAC ATT ATT GTT TTC TTC AGC (SEQ ID NO:27), which encodes the C-terminal PspA amino acids 250 to 257 followed by a PstI site. Digestion of the PCR product with EcoRI and PstI and cloning into pYA3620 similarly digested with EcoRI and PstI will yield the desired in-frame fusion. A similar strategy can be used to clone the sequence encoding the PspA_{EF5668}-PSpA_{Rx1} fusion from pYA3605 into pYA3620.

### Example 7

### Superior Immune Response to PspA Antigen when Secreted Rather than Retained in Cytoplasm of Recombinant Attenuated Vaccine Strain

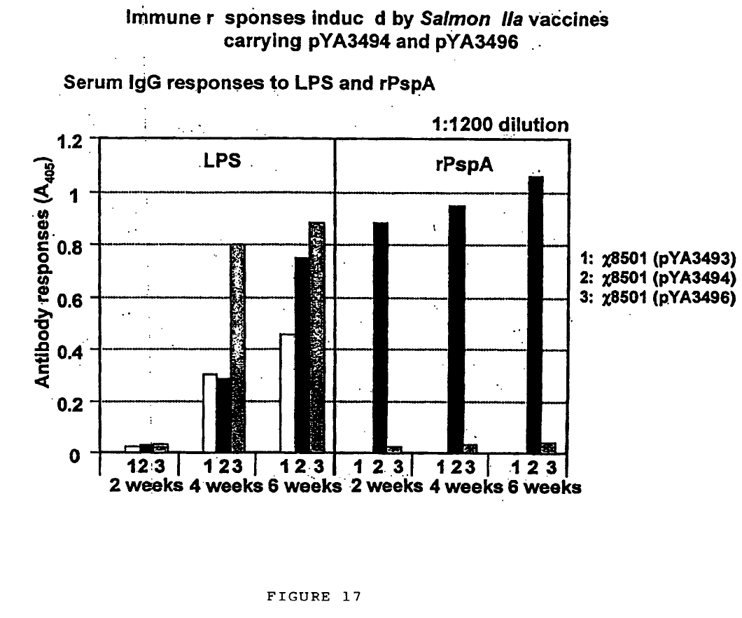

To raise antibodies to purified PspA protein, plasmid pYA3496, which encodes six His residues by alternate CAT and CAC codons preceded by the codons for Met, Gly and lie at the N-terminal end of the PspA protein (amino acids 3 to 257), was constructed. This enabled affinity purification using Ni²⁺-nitrilotriacetic acid-agarose and yielded purified PspA protein specifically recognized by the MAb Xi126. All of the His-PspA protein was retained within the cytoplasm of either E. coli K-12 stain ×6212 or the S. typhimurium UK-1 ×8501 vaccine strain and required lysis of the strain by sonication or by use of a French press for recovery. When ×8501 (pYA3496) was comparatively evaluated for immunogenicity to ×8501 (pYA3494), both strains induced an immune response to the LPS antigen; whereas, the immune response to PspA was substantial following immunization of BALB/c mice with ×8501 (pYA3494) and miniscule after immunization with ×8501 (pYA3496) (FIG. 17).

### Example 8

### Optimizing Antigen Gene Expression by Replacing Codons Infrequently Used by Salmonella for Highly Expressed Genes with Codons Preferentially Used by Salmonella for Highly Expressed Genes

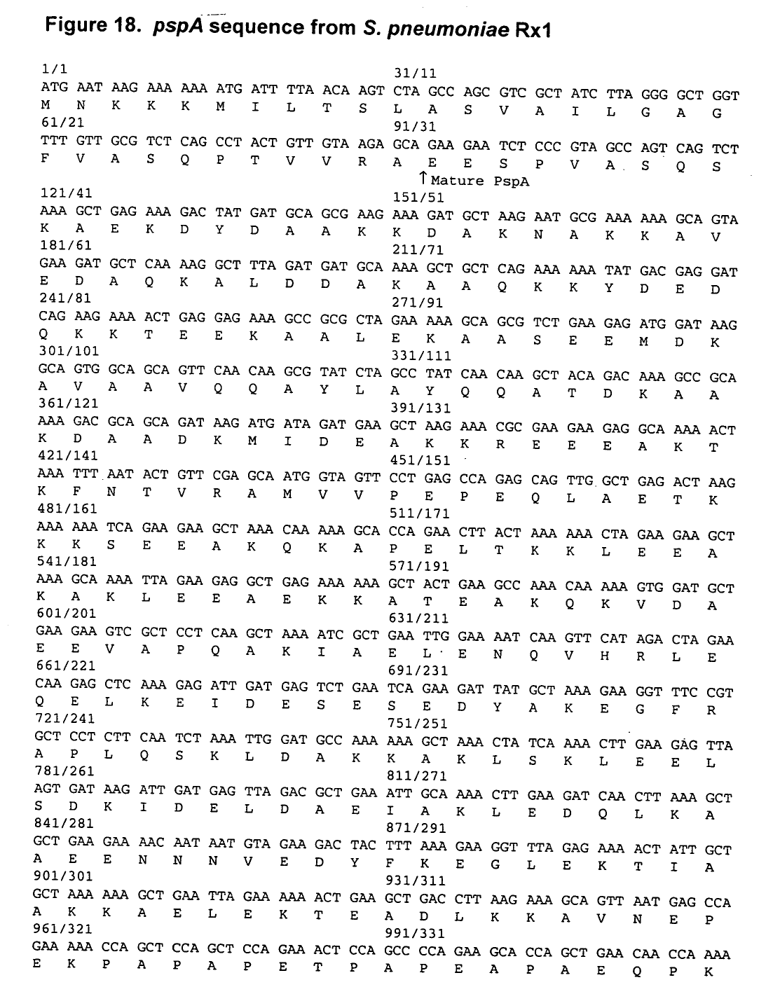

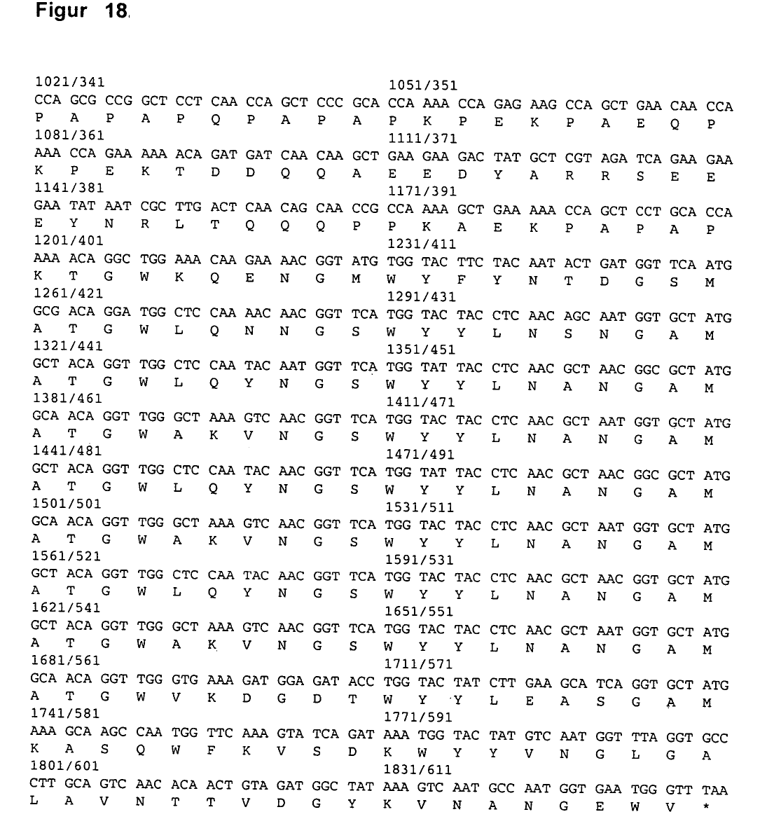

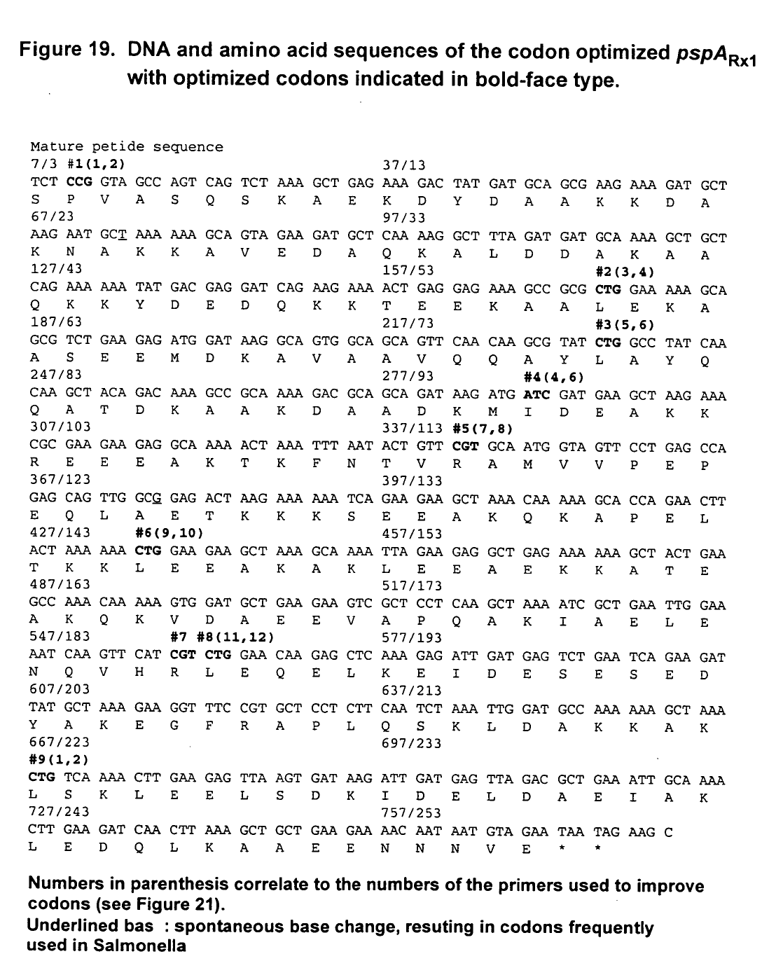

Codons for the amino acids leucine, arginine, isoleucine, glycine and proline used in genes of Streptococcus pneumoniae are often codons that strains of Escherichia coli and Salmonella infrequently do not include, especially, in genes that are highly expressed. For this reason, improved expression of genes encoding protective antigens, especially from gram-positive bacteria, can be achieved by eliminating (i.e., changing) any codons within genes encoding, for example, streptococcal protective antigens such as PspA and substituting in their place, codons for the same amino acids that are frequently used in Salmonella and E. coli highly expressed genes (Henaut and Danchin, In Escherichia coli and Salmonella Cellular and Molecular Biology, Vol. 2, 1996, Neidhardt et al., eds., ASM Press, Washington D.C., pp. 2047-2066; Gouy and Gautier, 1982, Nucleic Acids. Res. 10:7055-7074). In this way, expression of the protective antigen gene can be very much enhanced without altering the amino acid sequence of the antigen or the immune response induced to it. We have therefore modified the α-helical domain specifying 255 amino acids representing amino acids 3 to 257 of the mature Streptococcus pneumoniae Rx1 pspA gene as listed in FIG. 18 by replacing nine codons with codons for the same amino acids that are preferentially included in highly expressed genes in Salmonella. We employed a sequential codon replacement strategy using site-directed mutagenesis and PCR methodology for each specific codon or, in some cases, neighboring codon modifications at the same time with reassembly of the intact coding sequence with preferred codons for all amino acids. The nucleotide and amino acid sequences of the modified PspA Rx1 gene and gene product are presented in FIG. 19 with the modified codons indicated in boldface type. The entire DNA sequence was determined by DNA sequencing techniques and there were two base pair changes (underlined in FIG. 19) at the third position of two codons that continued to specify a codon from highly expressed genes for the same amino acid specified by the native PspA Rx1 sequence.

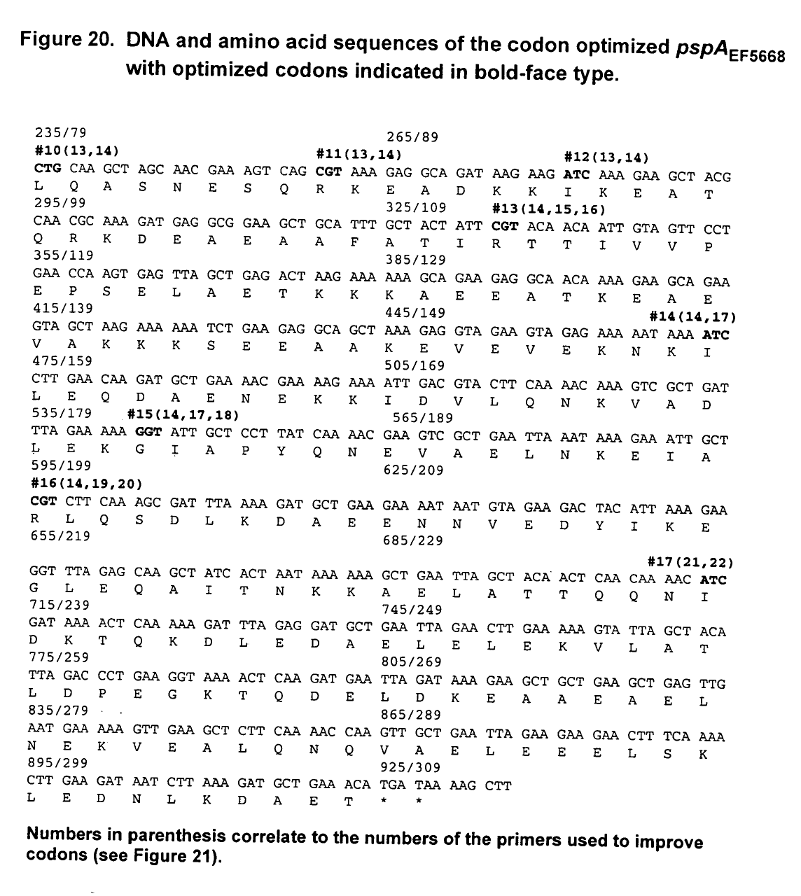

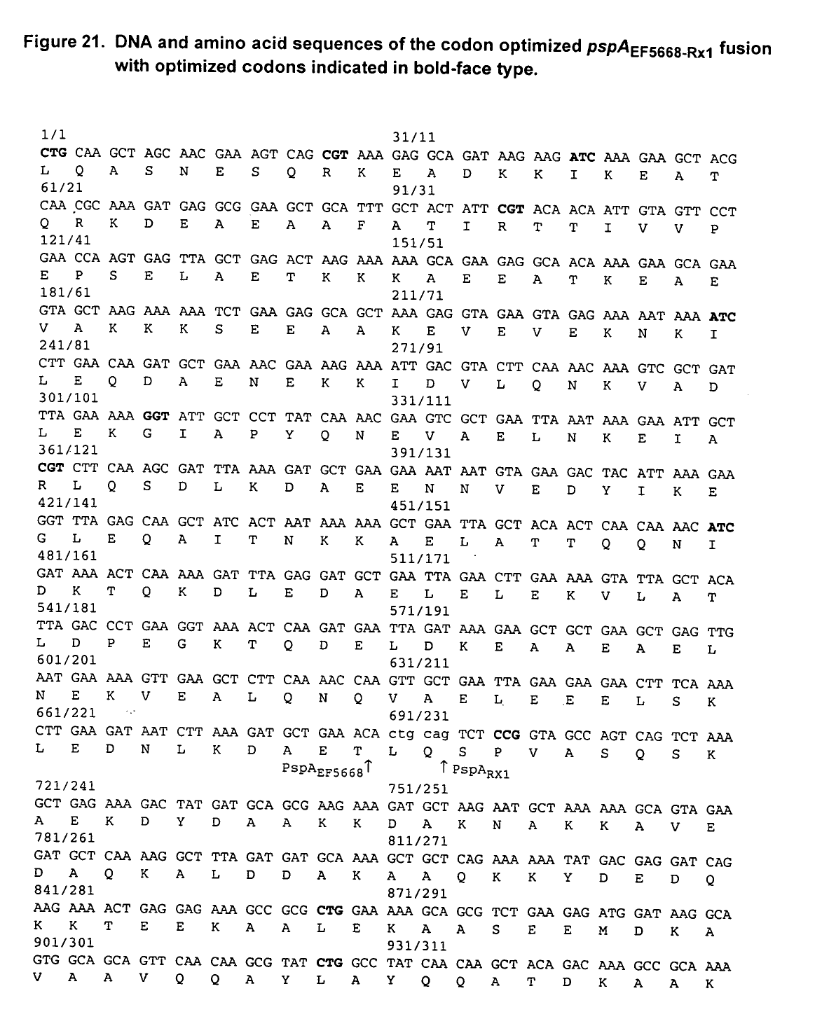

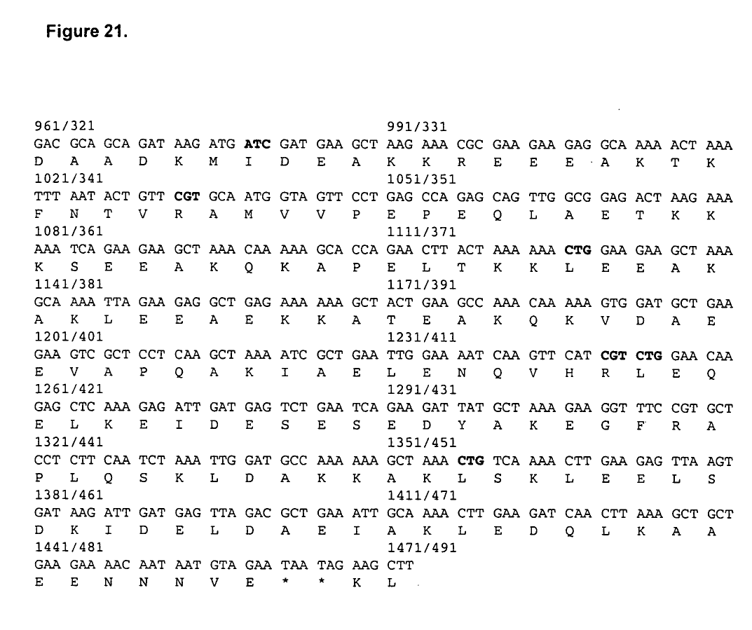

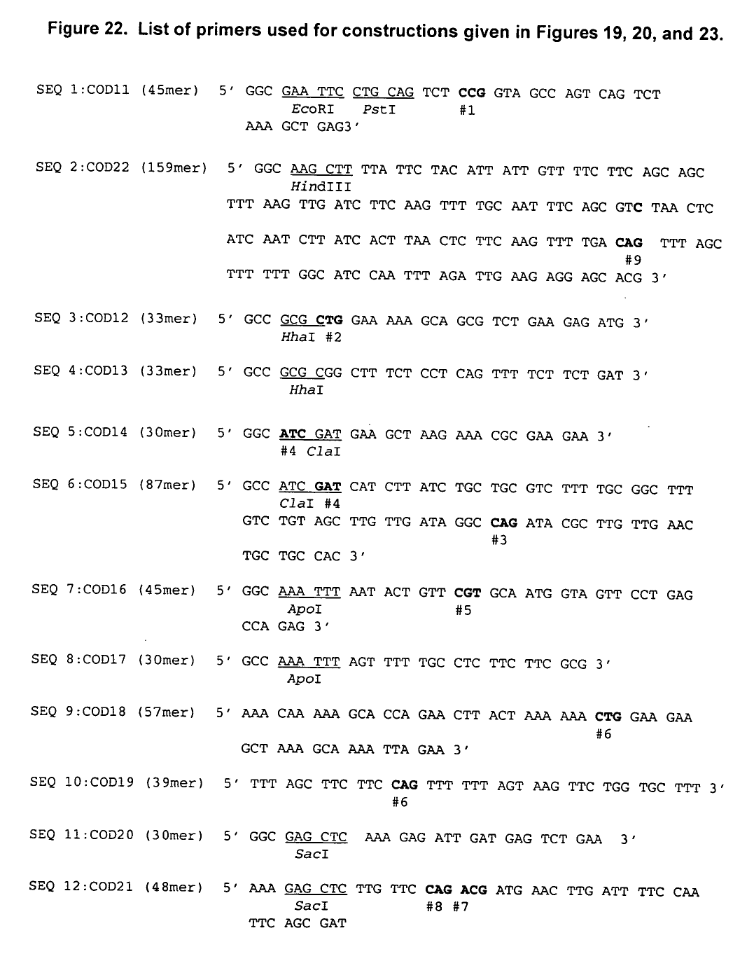

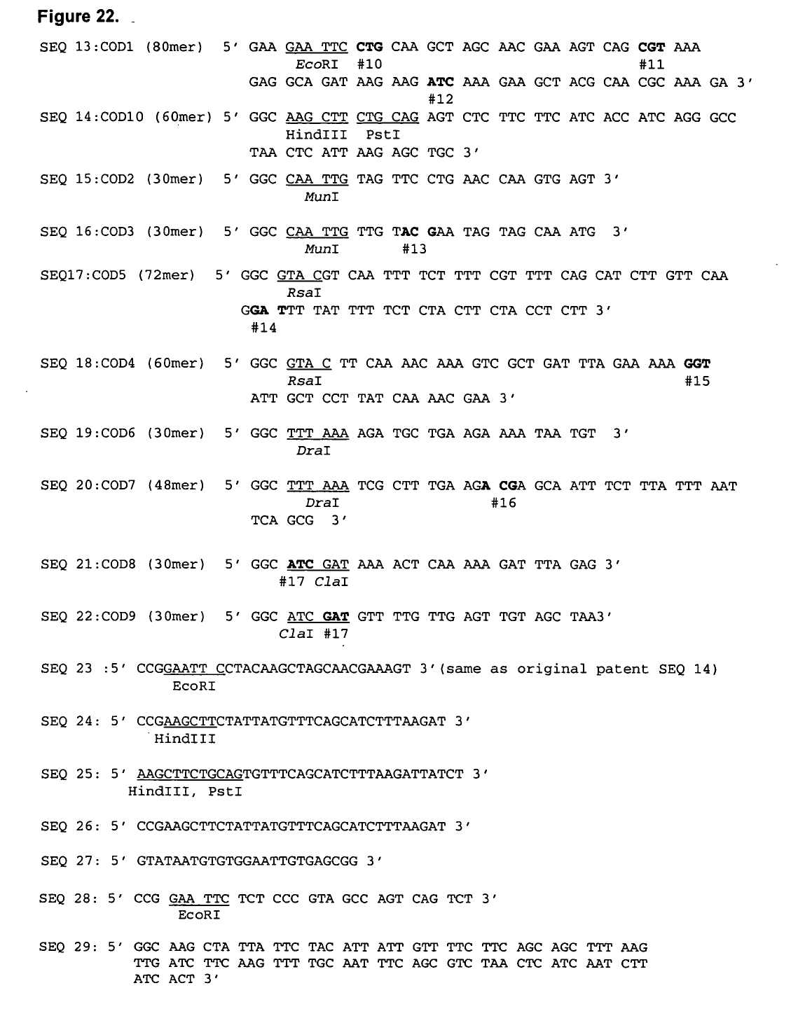

As indicated in Example 5, there are two major families of S. pneumoniae strains with regard to the immunogenic properties of the PspA protein. Strain Rx1 is in family 1 and we have selected S. pneumoniae strain EF5688 for the cloning of the α-helical domain as a representative of family 2. FIG. 20 provides the nucleotide and amino acid sequences of the modified PspA EF5688 gene and 229 amino acid gene product with the eight modified codons used in Salmonella highly expressed genes indicated in bold-face type. The α-helical domain of the EF5688 PspA protein represents amino acids 79 to 307 in the mature PspA protein (see FIG. 10). To facilitate presentation of the α-helical domains of both PspA proteins by the same vaccine strain, we constructed a fusion of the modified EF5688 (FIG. 20) and Rx1 (FIG. 19) sequences containing improved codons in the same way we used to make the fusion construct present in pYA3605 (FIG. 12) and whose nucleotide sequence is given in FIG. 13. The nucleotide and amino acid sequences of this modified fusion gene product with modified codons indicated in bold-face to optimize expression in Salmonella are presented in FIG. 21. A ctg cag sequence specifying a PstI restriction enzyme cleavage site was inserted by use of specified oligonucleotide primers between the C-terminal end of the PspA_{EF5688} sequence and the N-terminal end of the PspA_{Rx1} sequence (FIG. 21) to enable construction of the fusions. FIG. 22 lists the sequences in each of the oligonucleotide primers used to optimize codons (FIGS. 19 and 20), to construct the fusion and to construct recombinant vectors (see below and FIG. 23).

### Example 9

### Construction of Expression Plasmid Vectors With EF5688, Rx1 and EF5688-Rx1 Fusion α-Helical PspA Sequences Optimized for Expression due to Replacement of Codons Inefficiently Utilized in Highly Expressed Genes of Salmonella With Codons Preferentially Used by Salmonella for Expression of Highly Expressed Genes

During the course of constructing the codon-optimized pspA gene sequences as described in Example 8, it was discovered that there was a single base pair mutation in the Rx1 α-helical pspA sequence cloned in pYA3494 (Example 1 and FIG. 3), to result in a mutation that altered the C-terminal 22 amino acids. This was corrected (FIG. 23) in constructing pYA3634 using pYA3494 and the oligonucleotide sequence 2 (FIG. 22) with the corrected pspA Rx1 sequence as a replacement for pYA3494 (FIG. 3). To enable comparative studies on growth, genetic stability, physiological attributes, ability to colonize lymphoid tissues in vivo and to induce immune responses, we have constructed a set of six recombinant plasmids, three with the original α-helical pspA nucleotide sequences as found in S. pneumoniae strains Rx1 and EF5688 separately and as an EF5688-Rx1 fusion and three with the codon optimized improved sequences for pSPARx1 (FIG. 19), pSPA_{EF5688} (FIG. 20) and the pSpA_{EF5688-Rx1} fusion (FIG. 21). The construction of pYA3605 with the native pSPA_{EF5688-Rx1} fusion was diagramed in FIG. 12 with its complete nucleotide and amino acid sequence presented in FIG. 13 along with the N-terminal β-lactamase signal sequence. The construction of the new plasmid pYA3634 with the β-lactamase signal sequence fused to the native pspA_{Rx1} sequence (FIG. 19) is diagramed at the bottom of FIG. 23. The construction of each of the other four plasmids is diagramed in FIG. 23 with an indication of the vectors and oligonucleotide sequences (given in FIG. 22) used. These include pYA3623 with the native PSPA_{EF5688} sequence (FIG. 10), pYA3633 with the codon optimized improved PSPA_{EF5688} sequence (FIG. 20), pYA3635 with the codon optimized improved pSPA_{Rx1} sequence (FIG. 19) and pYA3636 with the codon optimized improved PspA_{EF5688-Rx1} fusion (FIG. 21) all fused to the β-lactamase signal sequence as contained in the original pYA3493 (FIG. 2).

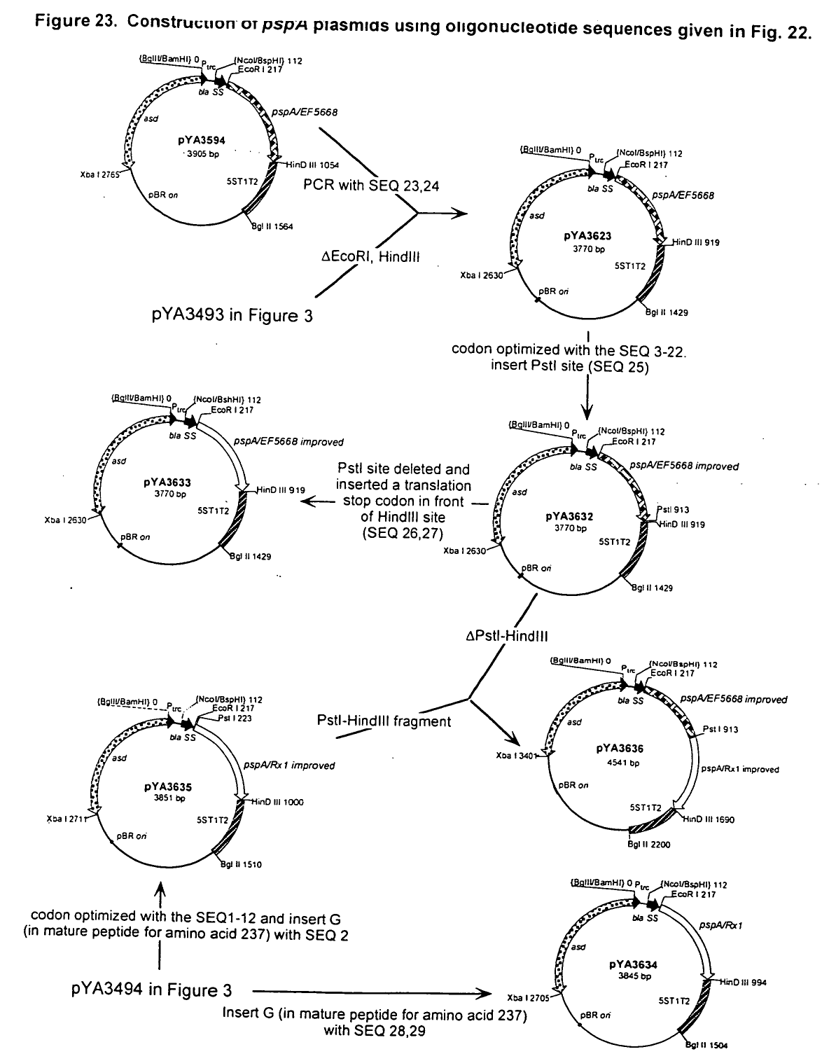

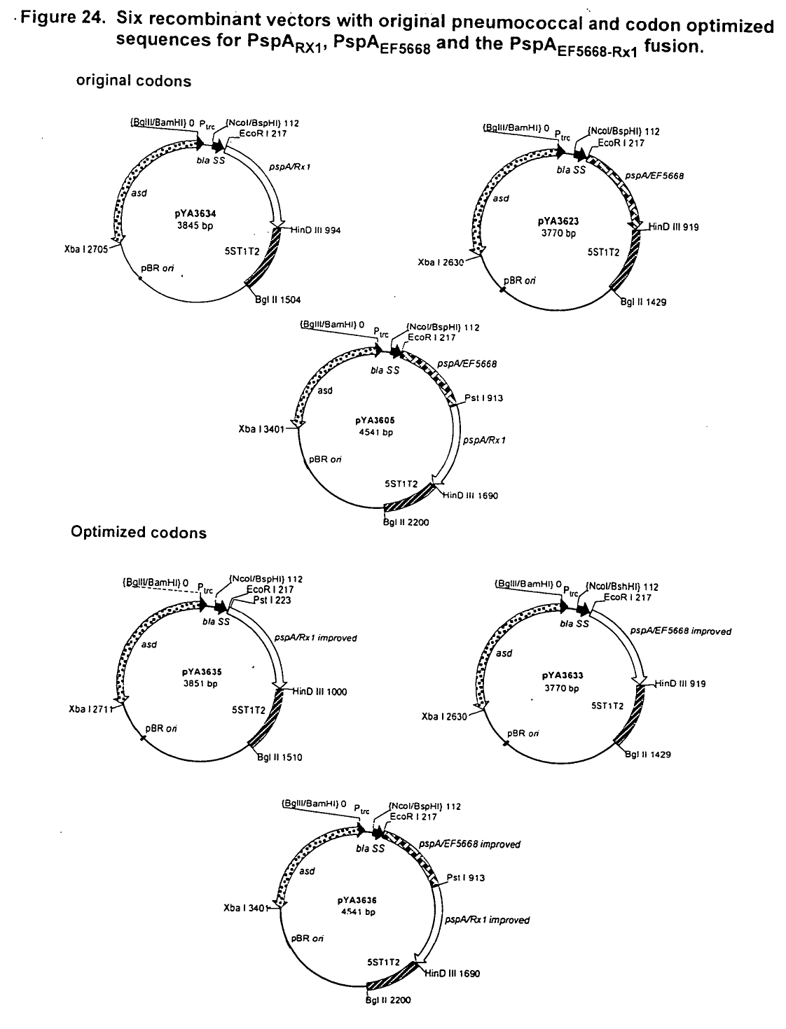

The resulting six expressions plasmids pYA3634, pYA3623, pYA3605, pYA3635, pYA3633 and pYA3636 are diagramed in FIG. 24. All six plasmids were electroporated first into the E. coli K-12 cloning host ×6212 (Table 1) with pYA232, a pSC101 plasmid with lacI gene, and also into the Δcrp Δasd S. typhimurium vaccine strain ×8501 (Table 1) with selection for Asd⁺ recombinants. These strains were fully characterized with regard to phenotypic and genotypic properties (see Example 2) and the presence of recombinant plasmids of the correct size, containing the intended pspA gene sequence (using PCR and DNA sequence analyses) and specifying synthesis of PspA proteins that would react in western blot analyses with antibodies specific for either or both the Rx1 and EF5688 PspA proteins. PspA synthesis in the ×6212 (pYA232) host required addition of the inducer IPTG to reverse repression of the P_{trc} promoter by the pYA232 specified Lacl repressor. These recombinant strains, listed Table 1, were catalogued, stocked and frozen at −70° C. for long-term storage.

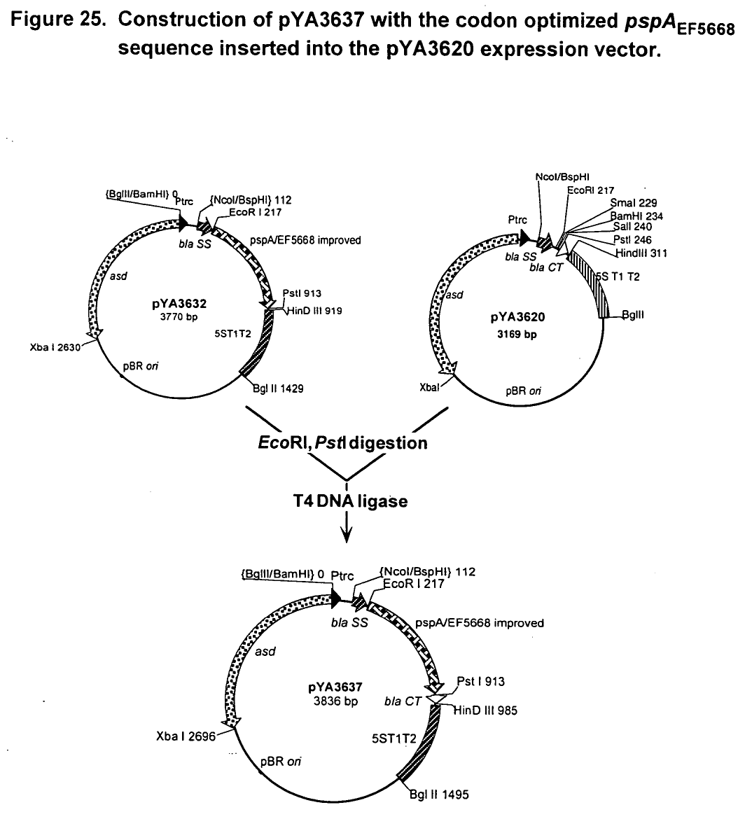

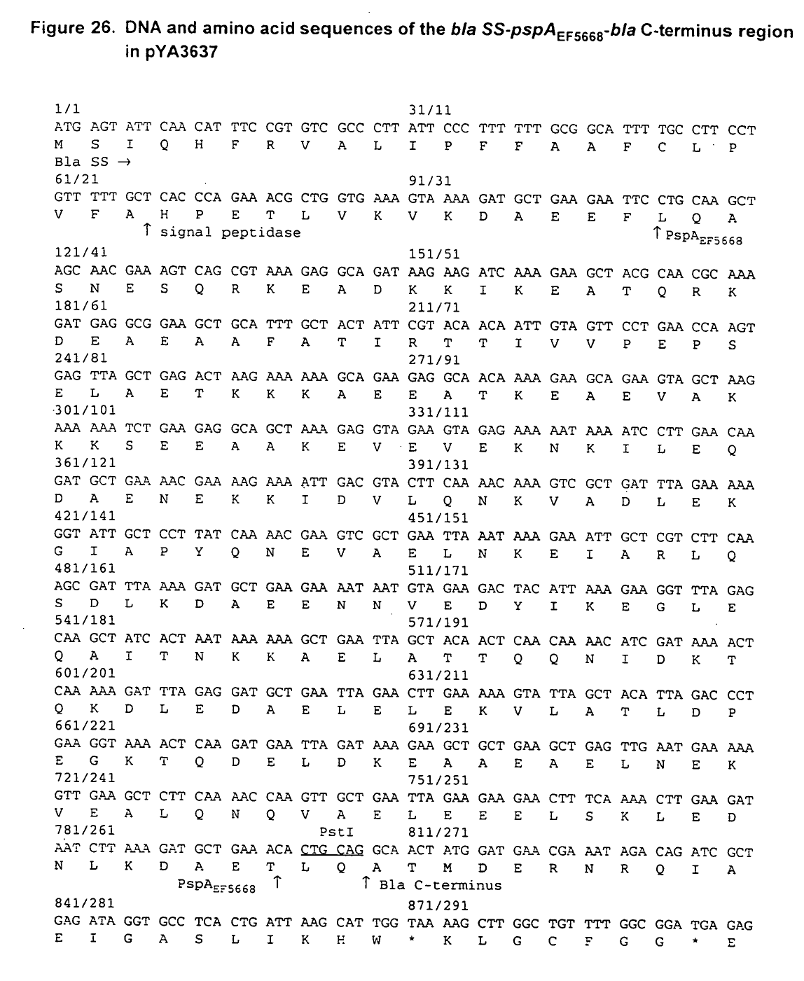

As described in Example 6, we constructed the Asd⁺ vector pYA3620 (FIG. 16) that possesses both the N-terminal signal sequence and the C-terminal end of the β-lactamase gene to investigate potential improved expression, secretion and/or stability of vaccine strains specifying synthesis of PspA proteins. As noted in the Example below, vaccine strains with the codon optimized improved PSPA_{EF5688} sequence (with or without fusion to the pspA_{Rx1} sequence) grew more slowly and exhibited some instability compared to vaccine strains expressing the native pneumococcal pspA_{EF5688} sequences. We have therefore constructed pYA3637 (FIG. 25) that contains the codon optimized improved pspA_{EF5688} sequence in the pYA3620 vector to evaluate potential improvements in expression, secretion and/or stability compared to a vaccine strain using the vector pYA3633 (FIGS. 23, 24) lacking the fused C-terminal β-lactamase peptide. FIG. 26 provides the entire nucleotide and amino acid sequences for the β-lactamase-signal sequence-improved pspA_{EF5688}-β-lactamase C-terminus fusion as present in pYA3637.

### Example 10

### Comparative Characterization of Recombinant Attenuated S. Typhimurium Vaccine Strains Specifying PspA Fusion and Non-Fusion Proteins Specified by Non-Codon Optimized and Codon Optimized DNA Sequences

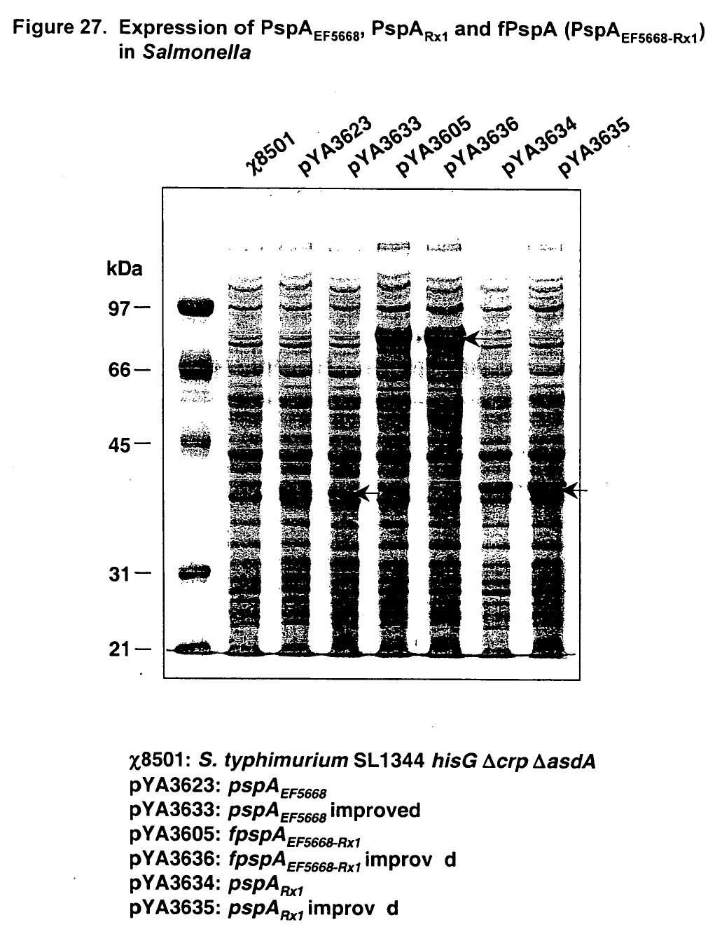

FIG. 27 presents results of a polyacrylamide gel stained with Coomassie brilliant blue after electrophoresis of proteins from the ×8501 parent and ×8501 with each of the six plasmids. The results clearly demonstrate synthesis of PspA proteins of the expected sizes and in every case reveal that the ×8501 strains with plasmids pYA3633, pYA3636 and pYA3635 with the codon optimized improved sequences for synthesis of the PspA_{EF5688}, PspA_{EF5688-Rx1} fusion and PspA_{Rx1} proteins overproduced these proteins in comparison with the ×8501 strains with the plasmids encoding PspA proteins specified by the native pneumococcal nucleotide sequences. All six strains were then evaluated, in comparison to ×8501 (pYA3493) as the vector control, for plasmid stability by growth in LB broth containing DAP with 1 to 1000 dilutions of cultures each day for five days. After 50 generations of growth, cultures were diluted and plated on LB agar containing DAP and replica plating was used to determine the frequency of cells that still possessed the Asd⁺ vector. For all six constructs specifying PspA proteins, some 10 isolates were grown in LB broth in the presence of DAP and evaluated for the synthesis of PspA proteins of the expected molecular size and reacting with antibodies to either or both the Rx1 or EF5688 PspA protein antigens. Five of seven strains retained the Asd⁺ vectors during 50 generations of growth under permissive conditions in the presence of DAP. The exceptions were ×8501 (pYA3633) and ×8501 (pYA3636) where there was significant loss of Asd⁺ vectors on day 3 and thereafter. Similarly, most isolates tested continued to synthesize PspA proteins of the correct size with the exception of the ×8501 (pYA3633) and ×8501 (pYA3636) strains both specifying codon optimized PspA proteins. In accord with these observations, it was determined that ×8501 (pYA3633) and especially ×8501 (pYA3636) grew significantly more slowly than either ×8501 (pYA3623) or ×8501 (pYA3605) with the original pneumococcal non-codon optimized sequences. This result is most likely due to the fact that the strains possessing the plasmids pYA3633 and pYA3636 with codon optimized pspA gene sequences produced much more PspA protein than did ×8501 strains with pYA3634 and pYA3623 having the non-codon optimized sequences as originally cloned from S. pneumoniae strains Rx1 and EF5688, respectively (see FIG. 27 for this evidence). This difference in growth rate was largest in comparisons between ×8501 (pYA3605) with the non-codon optimized fusion sequence and X8501 (pYA3636) that has the codon optimized DNA sequence encoding the PspA EF5688-Rx1 fusion protein. In this case, growth of the latter strain was at half of the rate of the former strain. Interestingly, the Rx1 encoded PspA protein, whether specified by the non-codon optimized sequence (pYA3634) or the codon optimized sequence (pYA3635), was well tolerated by the Salmonella vaccine strain ×8501 with complete stability in the maintenance of Asd⁺ vectors and the synthesis of PspA proteins (see FIG. 27).

In past studies with recombinant attenuated Salmonella antigen delivery vaccines, it has been observed that immune responses are nearly proportional to the amount of antigen production by the recombinant attenuated Salmonella vaccine. In other words, more antigens produced by the vaccine strain induces higher levels of immune responses. This relationship, however, breaks down if the level of antigen synthesis significantly inhibits the rate of growth and multiplication of the vaccine strain since, in this case, the recombinant attenuated Salmonella vaccine has reduced capacity to invade and colonize internal lymphoid tissues that constitute sites in which immune responses are induced.

### Example 11

### Construction of Vaccine Strains Expressing Protective Bacterial Antigens to Optimize Colonization of Lymphoid Tissues In Vivo and Induction of High Level Immune Responses

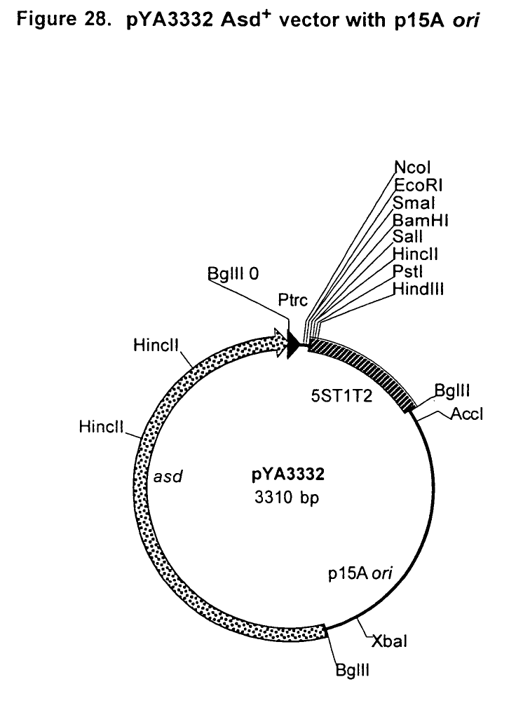

There are at least two possible solutions to the problem of vaccine strain instability as described in Example 10. In one case, it is possible to re-clone the codon optimized sequences using PCR methods and the oligonucleotide primers listed in FIG. 22 into the Asd⁺ vector pYA3332 (FIG. 28) that possesses the p15A ori specifying a lower plasmid copy number per bacterial cell than specified by the plasmid derivatives used above that were derived from pYA3342 (FIG. 2) that possesses the pBR ori. It should be noted, that pYA3332 and pYA3342 possess nearly identical structures except for the origin of replication. They have the same promoter, multiple cloning site and transcription terminator sequences as indicated in FIG. 2. The introduction of these pYA3332 PspA-specifying plasmids into ×8501 would be expected to result in strains that would grow more rapidly, and more stably maintain the Asd⁺ plasmids but synthesize a somewhat reduced amount of the PspA proteins specified by the codon optimized sequences. This is because the amount of gene product synthesized is proportional to the gene copy number and the fact that the gene copy number is significantly lower on vectors with p15A ori than on vectors with the pBR ori. The level of PspA synthesis would therefore be expected to be about the same as in ×8501 strains possessing Asd⁺ vectors with non-codon optimized pspA sequences encoding the PspA antigen synthesized. These recombinant attenuated vaccine strains would be fully characterized phenotypically and genotypically for genetic stability, growth rate, expression of PspA antigen, colonization of lymphoid tissues and induction of immune responses, all in comparison with recombinant attenuated Salmonella vaccine strains containing recombinant Asd⁺ vectors with the pBR ori. Although this approach would likely yield recombinant attenuated vaccine strains that would colonize lymphoid tissues and induce immune responses that might be superior to those induced by ×8501 (pYA3494) (see Example 4), there is an even better way to achieve high-level colonization of lymphoid tissues and induction of superior immune responses.

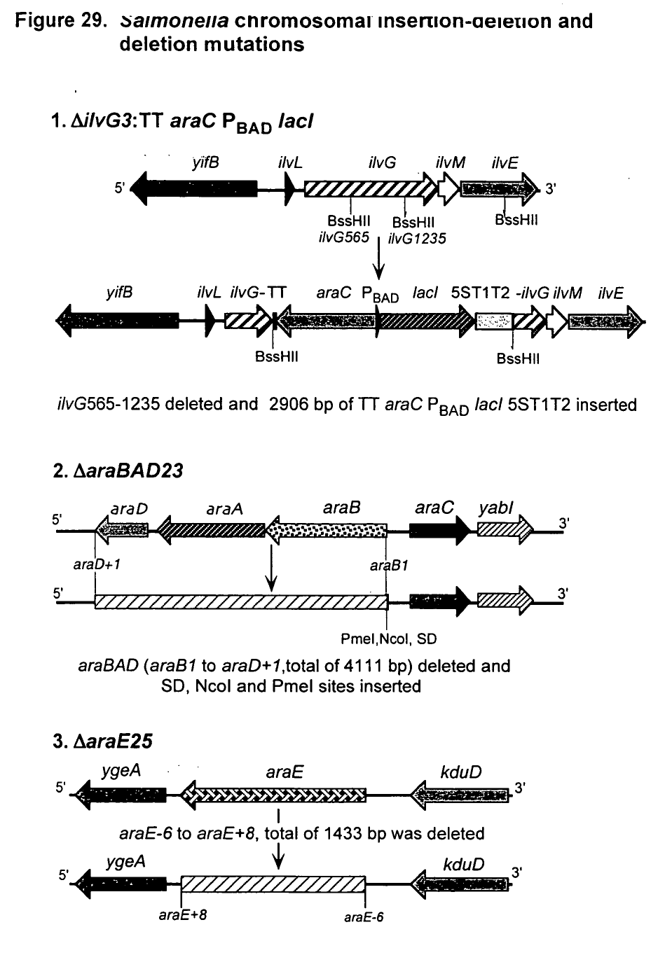

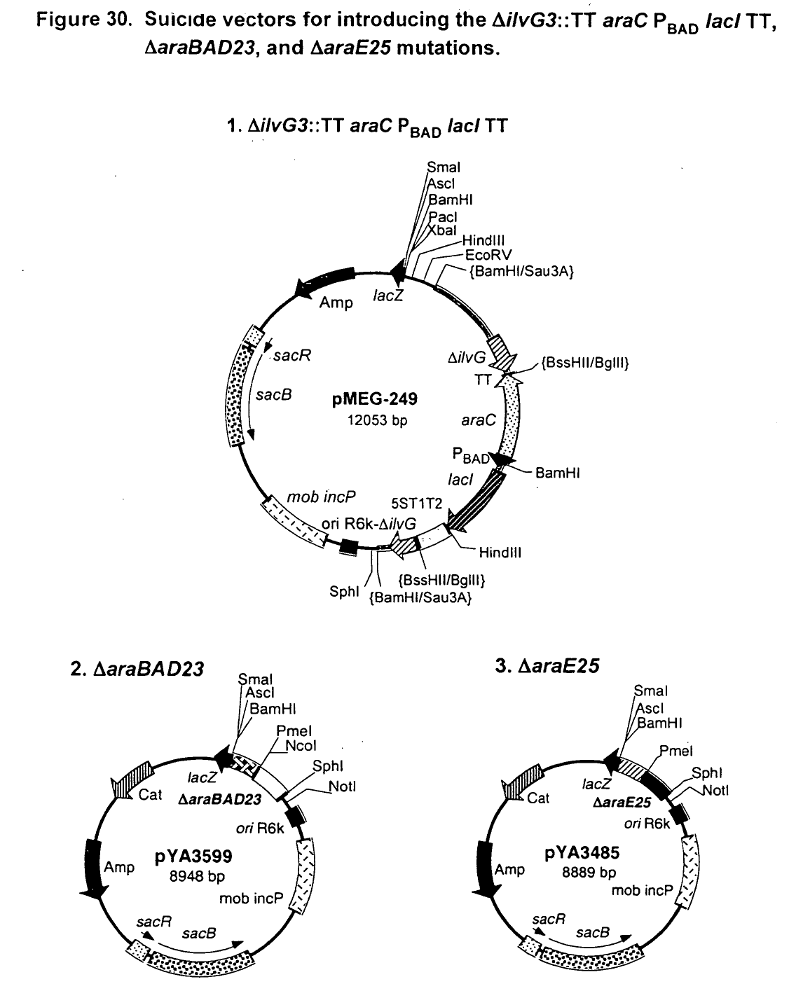

All of the Asd⁺ recombinant vectors cause expression via transcription from the P_{trc} promoter that can be completely repressed to block transcription by the presence of the Lacl repressor protein. This was proven to be so when each of the Asd⁺ plasmids was introduced into ×6212 (pyA232) and PspA synthesis shown to be dependent on addition of IPTG to cause depression of transcription from P_{trc} (Example 9). What is desired, is a recombinant attenuated vaccine strain that would not synthesize PspA antigen due to non-transcription from P_{trc} during growth in vitro and in the initial stages following immunization when the vaccine strain is invading into the GALT and commencing to colonize internal lymphoid tissues after which synthesis of PspA antigen is desired to induce immune responses. To facilitate delayed expression of DNA sequences encoded on Asd⁺ vectors downstream from P_{trc}, we have employed the araCP_{BAD} activator-promoter system (Guzman et al., 1995, J. Bacteriol. 177:4121-4130). In this system, a promoterless gene encoding the Lacl protein is fused to the P_{BAD} promoter, so that transcription is dependent on the presence of arabinose that activates the araC gene product to positively cause transcription from P_{BAD}. Interestingly, there is essentially no free arabinose in animal tissues such that following colonization of lymphoid tissues transcription of the lad gene ceases with the Lacl repressor decreasing in concentration by half after every cell division to gradually lead to derepression in the expression of gene sequences under the control of P_{trc} on the Asd⁺ plasmid vectors. The araCP_{BAD} lacl construction has therefore been inserted into the ilvG gene within the bacterial chromosome. The ΔilvG3:: TT araC P_{BAD} lad construction present in ×8623 (Table 1) is diagramed in FIG. 29 and the suicide vector for its introduction into the bacterial chromosome of vaccine strains such as ×8501 by methods described by Kang et al. (2002, J. Bacteriol. 184:307-312) is diagramed in FIG. 30. The delayed synthesis of protective antigens in vaccine strains with the ΔilvG3:: TT araC P_{BAD} lacl construction can be extended if the vaccine strain is unable to catabolically break down arabinose (Guzman et al., 1995, 177:4121-4130). This can be accomplished by the introduction of the ΔaraBAD23 mutation (FIG. 29) present in ×8767 (Table 1) using the suicide vector diagramed in FIG. 30. Further delay can be achieved by introducing the ΔaraE25 mutation present in ×8477 (Table 1) that both reduces the rate of uptake and the rate of loss of arabinose from the cytoplasm of vaccine cells. The ΔaraE25 construction is also diagramed in FIG. 29 and the suicide vector for its introduction into the chromosome of vaccine strains is also diagramed in FIG. 30. The methods for generating defined deletion mutations and their introduction into the chromosome using suicide vector technologies was described in Example 2 and more extensively in the manuscript by Kang et al (2002, J. Bacteriol. 184:307-312).

Following construction of a ×8501 derivative with the ΔilvG3:: TT araC P_{BAD} lacl, ΔaraBAD23 and araE25 mutations, recombinant Asd⁺ plasmid vectors such as pYA3633, pYA3635 and pYA3636 with codon optimized sequences encoding PspA antigens can be introduced by electroporation. These recombinant attenuated vaccine strains would be fully characterized phenotypically and genotypically for genetic stability, growth rate, non-expression of PspA antigen when the strain is grown in the presence of arabinose and gradual ability to synthesize PspA antigen as a consequence of cell division after removal of arabinose from the growth medium, colonization of lymphoid tissues and induction of immune responses. Appropriate control strains would be included for comparison.

### Example 12

### Construction of Vaccine Strains Specifying Synthesis of the α-Helical Domain of the Streptococcus Pneumoniae PsaA Protein to be Secreted, in Part, by Recombinant Attenuated Salmonella Vaccines to Elicit Superior Immune Responses Against S. Pneumoniae Infection in Immunized Individuals

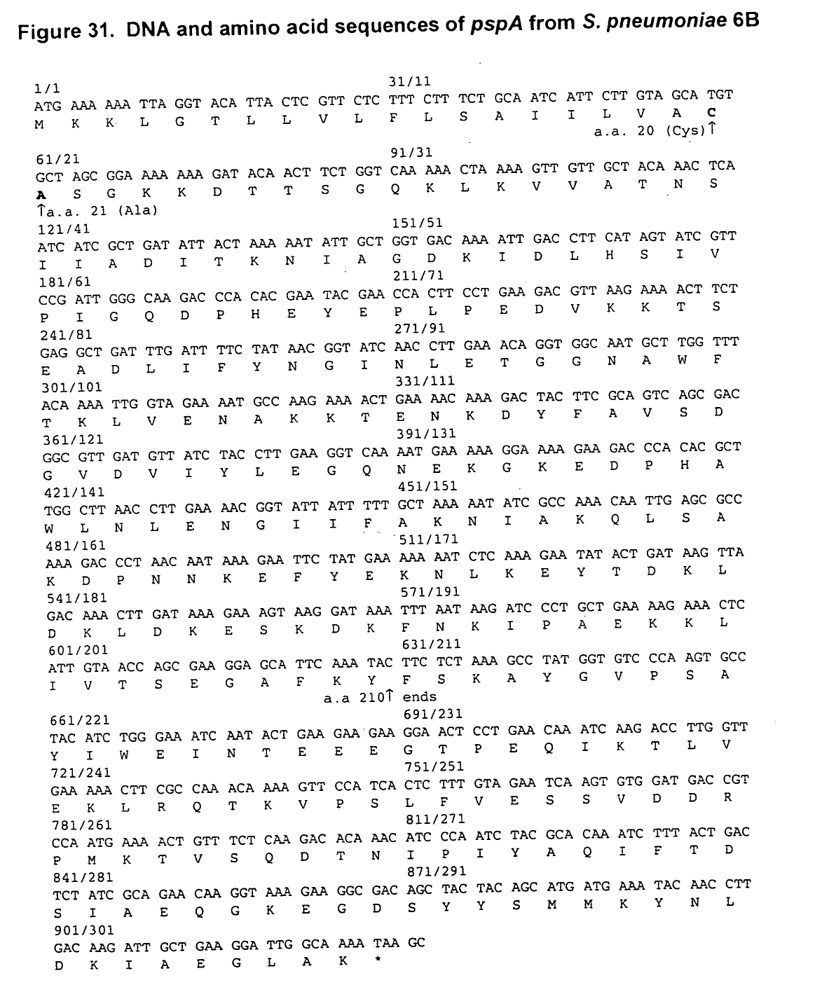

In terms of an effective vaccine to prevent infection of humans with diverse Streptococcus pneumoniae serotypes, the synthesis of the α-helical domains of the EF5688 and Rx1 PspA proteins is likely to induce significant protective immune responses. On the other hand, the inclusion of an additional antigen to be synthesized by the vaccine strain would further enhance vaccine efficacy. The PsaA antigen, encoded in the genome of S. pneumoniae 6B, a most prevalent pneumococcal serotype (Butler et al., 1995, J. Infect. Dis. 171:885-889; Jorgenson et al., 1991, J. Infect. Dis. 163:644-646), is a lipidated 32.5 kDa antigen with an N-terminal Cys residue generated after cleavage of the 19 amino acid signal sequence (De et al., 2000, Vaccine 18:1811-1821). The surface PsaA protein is an adhesin used by pneumococci to colonize in the nasal pharynx. Its synthesis is essential for S. pneumoniae virulence (Berry and Paton, 1996, Infect. Immun. 64:5255-5262) and this protein antigen can induce protective immunity to pneumococcus challenge (Briles et al., 2000, Infect. Immun. 68:796-800; Briles et al., 2000, Vaccine 18:1707-1711; Talkington, 1996, Microb. Pathog. 21:17-22). Because of these properties and functions, immunity to PsaA is more effective in blocking colonization in the nasal pharynx and immunity to PspA more effective in blocking systemic infection (Briles et al., 2000, Infect. Immun. 68:796-800). An additional beneficial feature of including the PsaA antigen for expression by a recombinant attenuated Salmonella vaccine is the fact that the amino acid sequence of this protein seems to be almost invariant in some eight psaA genes sequenced from diverse pneumococcus strains to date (analyzed using GenBank deposited sequences). Structural analysis of the PsaA protein causes us to express an α-helical hydrophilic segment commencing with either amino acid 20 (Cys) or 21 (Ala) and ending with amino acid 210 as diagramed in FIG. 31. All four non optimal codons for expression in Salmonella would be optimized for high-level expression in Salmonella by site directed mutagenesis and PCR methodologies and the codon optimized sequence encoding the α-helical domain of PsaA cloned, using suitable oligonucleotide primers, into either pYA3493 (FIG. 2) and/or pYA3620 (FIGS. 15 and 16). These plasmid constructs would be introduced into the ×8501 derivative with ΔilvG3:: TT araC P_{BAD} lacl, ΔaraBAD23 and ΔaraE25 mutations. These constructs would be fully evaluated for phenotype, genotype, stability of plasmid vector, rate of growth, inability to synthesize the PsaA protein when grown in the presence of arabinose and gradual ability following cell division in the absence of arabinose to commence PsaA synthesis, to colonize lymphoid tissues after oral immunization of mice and to induce significant immune responses including protective immunity to challenge with virulent S. pneumoniae strains. All of these procedures have been described in proceeding examples. Upon completion of these evaluations, a decision would be made whether to specify the codon optimized PspA EF5688-Rx1 fusion and the codon optimized PsaA sequence as a fusion in a single vector in the vaccine strains or by two separate vaccine strains that would be used as a vaccine mixture.

### Example 13

### Construction of Vaccine Strains Specifying Synthesis of Surface Protein Antigens of Various Streptococcus Species and From Other Gram-Positive Bacterial Pathogens to Enable Secretion, in Part, of the α-Helical Domains of these Proteins by Recombinant Attenuated Salmonella Vaccines to Elicit Superior Immune Responses in Immunized Individuals

There is great similarity in the structural properties of surface protein antigens synthesized by a diversity of Streptococcus species. These surface protein antigens may serve as adhesins to host cell surfaces, interact with extracellular matrix proteins, exhibit antiphagocytic activity, bind choline, and presumably provide additional functions ( ). These proteins all possess highly immunogenic α-helical domains near their N-terminus following cleavage of the signal sequence ( ). These gene products include the choline-binding proteins found on strains of S. pneumoniae, the M proteins on the surface of Group A S. pyogenes strains, surface proteins on the surface of Group B streptococci, the M protein on the surface of S. equi that is responsible for much of the pathology associated with the disease strangles of horses, the antigen I/II of S. mutans and the SpaA antigen of S. sobrinus. In these regards, we have had extensive experience in inducing immune responses using recombinant attenuated Salmonella to the SpaA protein of S. sobrinus (Holt et al., 1982, Infect. Immun. 38:147-156; Curtiss, 1986, J. Dent Res. 65:1034-1045) and the M protein of S. equi (Galán et al., 1989, In D. G. Powell (ed.), Equine Infectious Diseases V, Proceedings of the Fifth International Congress, The University Press of Kentucky, Lexington, Ky.). Others have used attenuated Salmonella to induce immune responses to surface antigens from S. pyogenes (Poirier et al., 1988. J. Exp. Med. 168:25-32) and S. mutans (Huang et al., 2000, Infect. Immun. 68:1549-1556).

Previous difficulties in constructing stable immunogenetic recombinant attenuated Salmonella vaccines expressing surface antigens of gram-positive bacterial pathogens could be due, in part, to the inability of the gram-negative Salmonella antigen delivery vector to efficiently synthesize and export gram-positive bacterial protein surface antigens in the absence of any instability and toxicity to the vaccine construct. In this regard, the gram-positive signal sequence either does not interact efficiently with or is not processed efficiently by the protein secretion system in gram-negative E. coli and S. typhimurium strains (Nayak et al., 1997, Infect. Immun. 66:3744-3751). The deletion of the signal sequence for the exported gram-positive protein and its replacement with a highly expressed and very efficient signal sequence whose function is optimal in gram-negative enteric bacteria can therefore be predicted to substantially improve the stability and the immunogenetic efficacy of these constructed vaccines as has been demonstrated for the α-helical domain of the S. pneumoniae PspA protein (see Example 4). Thus the use of plasmid vectors such as pYA3493 (FIG. 2) and pYA3620 (FIGS. 15 and 16) to clone codon optimized DNA sequences encoding α-helical domains of surface protein antigens from gram-positive bacterial pathogens such as the species of Streptococcus listed above, Erysipelothrix rhusiopathiae, Bacillus anthracis, Corynebacterium diphtheriae, Clostridium species, etc. for expression and delivery by recombinant attenuated Salmonella vaccine strains would likely generate vaccines that would be efficacious due to their stability and ability to synthesize and deliver the recombinant vector-encoded protective antigen, all contributing to high immunogenicity.

### Example 14

### Construction of Recombinant Attenuated S. Paratyphi A and S. Typhi Vaccines to Induce Protective Immunity to Humans to Prevent Infections Caused by Streptococcus Pneumoniae, other Streptococcus Species and Other Gram-Positive Bacterial Pathogens

Evaluation and validation of recombinant attenuated Salmonella vaccines for delivery of antigens specified by DNA sequences encoding protective antigens from other pathogens invariably makes use of strains of S. typhimurium and immunization studies in mice. This is because S. typhimurium induces a disease in mice that is similar to the enteric fever and typhoid fever diseases caused by S. paratyphi A and S. typhi in humans. Unfortunately, S. paratyphi A and S. typhi are human host specific, are unable to infect mice and are rapidly cleared as though they were innocuous strains of bacteria. Since protecting mice against pneumococcal disease does not constitute a worthwhile long-term objective, we have constructed a diversity of S. paratyphi A and S. typhi attenuated strains that can be used as antigen delivery vectors for recombinant antigens that were initially presented to mice by attenuated strains of S. typhimurium to evaluate safety and efficacy. To optimize immunogenicity, these attenuated S. paratyphi A and S. typhi strains possess an RpoS⁺ phenotype (Nickerson and Curtiss, 1997, Infect. Immun. 65:1814-1823; U.S. Pat. Nos. 6,024,961 and 6,383,496). Many of the genetic means to fully attenuate S. typhimurium for mice and to render them highly immunogenic are inadequately attenuating in S. typhi administered to human volunteers. For this reason, we have been developing improved means to attenuate S. paratyphi A and S. typhi to use as antigen delivery vectors for humans. Although some of the S. paratyphi A and S. typhi strains with mutations eliminating functions of the phoPQ operon that might be suitable and safe as vaccine vectors for humans are listed in Table 1, other improved attenuation strategies that we will use for S. paratyphi A and S. typhi are included in a simultaneously submitted patent application entitled “Regulated attenuation of live vaccines to enhance cross protective immunogenicity”. All of these attenuated S. paratyphi A and S. typhi antigen delivery vectors for humans also possess the ΔasdA16 mutation (see Table 1) to enable immediate use of the constructed recombinant Asd⁺ vectors evaluated in S. typhimurium in mice using the standard balanced-lethal host-vector system (U.S. Pat. No. 5,672,345 and pending U.S. patent application entitled “Functional balanced-lethal host-vector systems”) to ensure long-term stability of the recombinant constructs with antigen synthesis in vivo in the absence of any exogenously applied selective pressure. Importantly, as a safety feature, all such recombinant attenuated Salmonella vaccines are completely sensitive to all antibiotics such that an individual experiencing an unexpected adverse consequence of immunization could be effectively treated with antimicrobial agents. This benefit does not exist if a live attenuated bacterial vaccine possesses genes for antibiotic resistance in its chromosome or more often on plasmid vectors specifying recombinant antigens. For this reason, regulatory agencies charged with evaluation of vaccines for safety and efficacy will almost always disallow presence of antibiotic resistance genes, especially if those antibiotics are potentially useful in treating infections by unattenuated bacteria of the same type as used as the vaccine host component. This benefit is generally precluded in using live recombinant attenuated viral vaccine vectors since there are few effective antiviral drugs available.

What is claimed is:

1. A vaccine comprising a live attenuated strain of pathogenic gram negative bacteria, wherein (a) the strain of bacteria comprises a first polynucleotide that encodes an antigen, (b) the first polynucleotide is operably linked to a second polynucleotide that encodes a secretion peptide, (c) the antigen is from a source that is different than the live attenuated strain of bacteria, and (d) the vaccine elicits a Th2-type immune response in a vertebrate.

2. The vaccine of claim 1 further comprising a balanced-lethal host-vector system.

3. The vaccine of claim 2 further comprising an environmental limitation viability system.

4. The vaccine of claim 2 wherein the live attenuated strain of bacteria comprises an inactivating mutation in the chromosomal gene encoding aspartate β-semialdehyde dehydrogenase (Asd) and a plasmid, wherein the plasmid comprises a polynucleotide that encodes a functional Asd and the first and second polynucleotides.

5. The vaccine of claim 1 wherein the strain of bacteria is a member of a group of bacteria selected from the group consisting of Enterobacteriaceae, Vibrionaceae, Francisellaceae, Legionallales, Pseudomonadacea and Pasteurellaceae.

6. The vaccine of claim 5 wherein the strain of bacteria is a member of the Enterobacteriaceae group.

7. The vaccine of claim 6 wherein the strain of bacteria is selected from the group consisting of Salmonella, Escherichia, Shigella and Yersinia.

8. The vaccine of claim 7 wherein the strain of bacteria is a Salmonella enterica.

9. The vaccine of claim 8 wherein the Salmonella enterica comprises an attenuating mutation in a gene encoding a protein selected from the group consisting of cyclic AMP receptor protein (“Crp”), adenylate cyclase (“Cya”), aspartate β-semialdehyde dehydrogenase (“Asd”) and DNA adenine methylase (“Dam”).

10. The vaccine of claim 9 wherein the protein is cyclic AMP receptor protein (“Crp”).

11. The vaccine of claim 1 wherein the antigen is a polypeptide produced by a pathogen.

12. The vaccine of claim 11 wherein the pathogen is selected from the group consisting of Streptococcus pneumoniae, Streptococcus pyogenes, Streptococcus group B, Streptococcus mutans, Streptococcus sobrinus, Streptococcus equi, Staphylococcus spp., Streptococcus mutans, Streptococcus sobrinus, Streptococcus equi, Erysipelothrix rhusiopathiae, Bacillus anthracis, Listeria monocytogenes, Mycobacterium leprae, Mycobacterium tuberculosis, Clostridium tetani, Clostridium botulinum, Clostridium difficile, Clostridium perfringens, Corynebacterium diphtheriae, and Mycoplasma spp.

13. The vaccine of claim 12 wherein the pathogen is Streptococcus pneumoniae.

14. The vaccine of claim 1 wherein the second polynucleotide encodes a sequence as set forth in SEQ ID NO:2 or SEQ ID NO:3.

15. The vaccine of claim 13 wherein the antigen is a pneumococcal surface protein A (“PspA”).

16. The vaccine of claim 15 wherein the second polynucleotide encodes a sequence as set forth in SEQ ID NO:2 or SEQ ID NO:3.

17. The vaccine of claim 16 wherein the first polynucleotide encodes a sequence as set forth in SEQ ID NO:1 and the second polynucleotide encodes a sequence as set forth in SEQ ID NO:2.

18. The vaccine of claim 17 wherein the strain of bacteria is a Salmonella enterica, which comprises (a) a mutation that renders the gene encoding cyclic AMP receptor protein inactive, (b) an inactivating mutation in the chromosomal gene encoding aspartate β-semialdehyde dehydrogenase (Asd) and (c) a plasmid, which comprises (i) the first polynucleotide, (ii) the second polynucleotide and (iii) a polynucleotide that encodes a functional Asd.

19. The vaccine of claim 13 wherein the first polynucleotide and the second polynucleotide together comprise the sequence as set forth in SEQ ID NO:20.

20. An immunogenic composition comprising a live attenuated strain of pathogenic gram negative bacteria, wherein (a) the strain of bacteria comprises a first polynucleotide that encodes an antigen, (b) the first polynucleotide is operably linked to a second polynucleotide that encodes a secretion peptide, (c) the antigen is from a source that is different than the live attenuated strain of bacteria, and (d) the immunogenic composition elicits a Th2-type immune response in a vertebrate.

21. The immunogenic composition of claim 20 further comprising a balanced-lethal host-vector system.

22. The immunogenic composition of claim 21 further comprising an environmental limitation viability system.

23. The immunogenic composition of claim 21 wherein the live attenuated strain of bacteria comprises an inactivating mutation in the chromosomal gene encoding aspartate β-semialdehyde dehydrogenase (Asd) and a plasmid, wherein the plasmid comprises a polynucleotide that encodes a functional Asd and the first and second polynucleotides.

24. The immunogenic composition of claim 20 wherein the strain of bacteria is a member of a group of bacteria selected from the group consisting of Enterobacteriaceae, Vibrionaceae, Francisellaceae, Legionallales, Pseudomonadacea and Pasteurellaceae.

25. The immunogenic composition of claim 24 wherein the strain of bacteria is a member of the Enterobacteriaceae group.

26. The immunogenic composition of claim 25 wherein the strain of bacteria is selected from the group consisting of Salmonella, Escherichia, Shigella and Yersinia.

27. The immunogenic composition of claim 26 wherein the strain of bacteria is a Salmonella enterica.

28. The immunogenic composition of claim 27 wherein the Salmonella enterica comprises an attenuating mutation in a gene encoding a protein selected from the group consisting of cyclic AMP receptor protein (“Crp”), adenylate cyclase (“Cya”), aspartate β-semialdehyde dehydrogenase (“Asd”) and DNA adenine methylase (“Dam”).

29. The immunogenic composition of claim 28 wherein the protein is cyclic AMP receptor protein (“Crp”).

30. The immunogenic composition of claim 20 wherein the antigen is a polypeptide produced by a pathogen.

31. The immunogenic composition of claim 30 wherein the pathogen is selected from the group consisting of Streptococcus pneumoniae, Streptococcus pyogenes, Streptococcus group B, Streptococcus mutans, Streptococcus sobrinus, Streptococcus equi, Staphylococcus spp., Erysipelothrix rhusiopathiae, Bacillus anthracis, Listeria monocytogenes, Mycobacterium leprae, Mycobacterium tuberculosis, Clostridium tetani, Clostridium botulinum, Clostridium difficile, Clostridium perfringens, Corynebacterium diphtheriae, and Mycoplasma spp.

32. The immunogenic composition of claim 31 wherein the pathogen is Streptococcus pneumoniae.

33. The immunogenic composition of claim 20 wherein the second polynucleotide encodes a sequence as set forth in SEQ ID NO:2 or SEQ ID NO:3.

34. The immunogenic composition of claim 32 wherein the antigen is a pneumococcal surface protein A (“PspA”).

35. The immunogenic composition of claim 34 wherein the second polynucleotide encodes a sequence as set forth in SEQ ID NO:2 or SEQ ID NO:3.

36. The immunogenic composition of claim 35 wherein the first polynucleotide encodes a sequence as set forth in SEQ ID NO:1 and the second polynucleotide encodes a sequence as set forth in SEQ ID NO:2.

37. The immunogenic composition of claim 36 wherein the strain of bacteria is a Salmonella enterica, which comprises (a) a mutation that renders the gene encoding cyclic AMP receptor protein inactive, (b) an inactivating mutation in the chromosomal gene encoding aspartate β-semialdehyde dehydrogenase (Asd) and (c) a plasmid, which comprises (i) the first polynucleotide, (ii) the second polynucleotide and (iii) a polynucleotide that encodes a functional Asd.

38. The immunogenic composition of claim 31 wherein the first polynucleotide and the second polynucleotide together comprise the sequence as set forth in SEQ ID NO:20.

39. A method of eliciting an immune response in a vertebrate, the method comprising administering a live attenuated strain of gram negative bacteria to said vertebrate, wherein (a) the strain of bacteria comprises a first polynucleotide that encodes an antigen, (b) the first polynucleotide is operably linked to a second polynucleotide that encodes a secretion peptide, (c) the antigen is from a source that is different than the live attenuated strain of bacteria, (d) the antigen is secreted from the strain of bacteria, and (e) the vertebrate produces IgG1 antibodies that specifically bind to the antigen.

40. The method of claim 39 wherein the strain of bacteria comprises a balanced-lethal host-vector system.

41. The method of claim 40 wherein the strain of bacteria comprises an environmental limitation viability system.

42. The method of claim 40 wherein the live attenuated strain of bacteria comprises an inactivating mutation in the chromosomal gene encoding aspartate β-semialdehyde dehydrogenase (Asd) and a plasmid, wherein the plasmid comprises a polynucleotide that encodes a functional Asd and the first and second polynucleotides.

43. The method of claim 39 wherein the strain of bacteria is a member of a group of bacteria selected from the group consisting of Enterobacteriaceae, Vibrionaceae, Francisellaceae, Legionallales, Pseudomonadacea and Pasteurellaceae.

44. The method of claim 43 wherein the strain of bacteria is a member of the Enterobacteriaceae group.

45. The method of claim 44 wherein the strain of bacteria is selected from the group consisting of Salmonella, Escherichia, Shigella and Yersinia.

46. The method of claim 45 wherein the strain of bacteria is a Salmonella enterica.

47. The method of claim 46 wherein the Salmonella enterica comprises an attenuating mutation in a gene encoding a protein selected from the group consisting of cyclic AMP receptor protein (“Crp”), adenylate cyclase (“Cya”), aspartate β-semialdehyde dehydrogenase (“Asd”) and DNA adenine methylase (“Dam”).

48. The method of claim 47 wherein the protein is cyclic AMP receptor protein (“Crp”).

49. The method of claim 39 wherein the antigen is a polypeptide produced by a pathogen.

50. The method of claim 49 wherein the pathogen is selected from the group consisting of Streptococcus pneumoniae, Streptococcus pyogenes, Streptococcus group B, Streptococcus mutans, Streptococcus sobrinus, Streptococcus equi, Staphylococcus spp., Erysipelothrix rhusiopathiae, Bacillus anthracis, Listeria monocytogenes, Mycobacterium leprae, Mycobacterium tuberculosis, Clostridium tetani, Clostridium botulinum, Clostridium difficile, Clostridium perfringens, Corynebacterium diphtheriae, and Mycoplasma spp.

51. The method of claim 50 wherein the pathogen is Streptococcus pneumoniae.

52. The method of claim 39 wherein the second polynucleotide encodes a sequence as set forth in SEQ ID NO:2 or SEQ ID NO:3.

53. The method of claim 51 wherein the antigen is a pneumococcal surface protein A (“PspA”).

54. The method of claim 53 wherein the second polynucleotide encodes a sequence as set forth in SEQ ID NO:2 or SEQ ID NO:3.

55. The method of claim 54 wherein the first polynucleotide encodes a sequence as set forth in SEQ ID NO:1 and the second polynucleotide encodes a sequence as set forth in SEQ ID NO:2.

56. The method of claim 55 wherein the strain of bacteria is a Salmonella enterica, which comprises (a) a mutation that renders the gene encoding cyclic AMP receptor protein inactive, (b) an inactivating mutation in the chromosomal gene encoding aspartate β-semialdehyde dehydrogenase (Asd) and (c) a plasmid, which comprises (i) the first polynucleotide, (ii) the second polynucleotide and (iii) a polynucleotide that encodes a functional Asd.

57. The method of claim 51 wherein the first polynucleotide and the second polynucleotide together comprise the sequence as set forth in SEQ ID NO:20.

58. The method of claim 56 wherein the vertebrate is a human and the strain of bacteria is administered orally.

59. A live attenuated strain of pathogenic bacteria comprising a first polynucleotide that encodes an antigen, wherein (a) the first polynucleotide is operably linked to a second polynucleotide that encodes a secretion peptide, (b) the antigen is from a source that is different than the live attenuated strain of bacteria, (e) the antigen is secreted from the live attenuated strain of pathogenic bacteria, and (f) the pathogenic bacteria is an Enterobacteriaceae.

60. The live attenuated strain of pathogenic bacteria of claim 59 further comprising a balanced-lethal host-vector system.

61. The live attenuated strain of pathogenic bacteria of claim 60 further comprising an environmental limitation viability system.

62. The live attenuated strain of pathogenic bacteria of claim 60 wherein the live attenuated strain of bacteria comprises an inactivating mutation in the chromosomal gene encoding aspartate β-semialdehyde dehydrogenase (Asd) and a plasmid, wherein the plasmid comprises a polynucleotide that encodes a functional Asd and the first and second polynucleotides.

63. The live attenuated strain of pathogenic bacteria of claim 59 wherein the strain of bacteria is selected from the list consisting of Salmonella, Shigella, Escherichia and Yersinia.

64. The live attenuated strain of pathogenic bacteria of claim 63 wherein the strain of bacteria is a Salmonella enterica.

65. The live attenuated strain of pathogenic bacteria of claim 64 wherein the Salmonella enterica contains a mutation that renders the gene encoding cyclic AMP receptor protein inactive.

66. The live attenuated strain of pathogenic bacteria of claim 59 wherein the antigen is a polypeptide produced by a pathogen.

67. The live attenuated strain of pathogenic bacteria of claim 66 wherein the pathogen is selected from the group consisting of Streptococcus pneumoniae, Streptococcus pyogenes, Streptococcus group B, Streptococcus mutans, Streptococcus sobrinus, Streptococcus equi, Staphylococcus spp., Erysipelothrix rhusiopathiae, Bacillus anthracis, Listeria monocytogenes, Mycobacterium leprae, Mycobacterium tuberculosis, Clostridium tetani, Clostridium botulinum, Clostridium difficile, Clostridium perfringens, Corynebacterium diphtheriae, and Mycoplasma spp.

68. The live attenuated strain of pathogenic bacteria of claim 67 wherein the pathogen is Streptococcus pneumoniae.

69. The live attenuated strain of pathogenic bacteria of claim 59 wherein the second polynucleotide encodes a sequence as set forth in SEQ ID NO:2 or SEQ ID NO:3.

70. The live attenuated strain of pathogenic bacteria of claim 68 wherein the antigen is a pneumococcal surface protein A (“PspA”).

71. The live attenuated strain of pathogenic bacteria of claim 70 wherein the second polynucleotide encodes a sequence as set forth in SEQ ID NO:2 or SEQ ID NO:3.

72. The live attenuated strain of pathogenic bacteria of claim 71 wherein the first polynucleotide encodes a sequence as set forth in SEQ ID NO:1 and the second polynucleotide encodes a sequence as set forth in SEQ ID NO:2.

73. The live attenuated strain of pathogenic bacteria of claim 72 wherein the strain of bacteria is a Salmonella enterica, which comprises (a) a mutation that renders the gene encoding cyclic AMP receptor protein inactive, (b) an inactivating mutation in the chromosomal gene encoding aspartate β-semialdehyde dehydrogenase (Asd) and (c) a plasmid, which comprises (i) the first polynucleotide, (ii) the second polynucleotide and (iii) a polynucleotide that encodes a functional Asd.

74. An immunogenic composition comprising a live attenuated strain of bacteria, wherein the strain of bacteria is an Enterobacteriaceae, which comprises a polynucleotide that encodes an antigen, wherein (i) the antigen is secreted from the cell, and (ii) the antigen is a polypeptide produced by a pathogen that is different than the live attenuated strain of bacteria.

75. The immunogenic composition of claim 74 wherein the strain of bacteria is a Salmonella.

76. The immunogenic composition of claim 75 wherein the pathogen is a Streptococcus bacteria.

77. The immunogenic composition of claim 76 wherein the antigen is a pneumococcal surface protein A (“PspA”).

78. The immunogenic composition of claim 77 wherein the strain of bacteria is Salmonella enterica.

79. The immunogenic composition of claim 78 wherein the immunogenic composition elicits the production in a vertebrate of IgG1 antibodies that bind to the pneumococcal surface protein A.

80. An immunogenic composition comprising a live attenuated strain of pathogenic gram negative bacteria, wherein (a) the strain of bacteria comprises (i) a first polynucleotide that encodes a first antigen, (ii) a second polynucleotide that encodes a second antigen and (iii) a third polynucleotide that encodes a secretion peptide, (b) the first antigen is from a serotype of a pathogen that is different from the live attenuated strain of pathogenic gram negative bacteria, (c) the second antigen is from a different serotype of the same pathogen from which the first antigen is derived and (d) the immunogenic composition elicits a Th2-type immune response in a vertebrate.

81. The immunogenic composition of claim 80 further comprising a balanced-lethal host-vector system.

82. The immunogenic composition of claim 81 further comprising an environmental limitation viability system.

83. The immunogenic composition of claim 81 wherein the live attenuated strain of bacteria comprises an inactivating mutation in the chromosomal gene encoding aspartate β-semialdehyde dehydrogenase (Asd) and a plasmid, wherein the plasmid comprises a polynucleotide that encodes a functional Asd and the first, second and third polynucleotides.

84. The immunogenic composition of claim 80 wherein the strain of bacteria is a member of a group of bacteria selected from the group consisting of Enterobacteriaceae, Vibrionaceae, Francisellaceae, Legionallales, Pseudomonadacea and Pasteurellaceae.

85. The immunogenic composition of claim 84 wherein the strain of bacteria is a member of the Enterobacteriaceae group.

86. The immunogenic composition of claim 85 wherein the strain of bacteria is selected from the group consisting of Salmonella, Escherichia, Shigella and Yersinia.

87. The immunogenic composition of claim 86 wherein the strain of bacteria is a Salmonella enterica.

88. The immunogenic composition of claim 87 wherein the Salmonella enterica comprises an attenuating mutation in a gene encoding a protein selected from the group consisting of cyclic AMP receptor protein (“Crp”), adenylate cyclase (“Cya”), aspartate β-semialdehyde dehydrogenase (“Asd”) and DNA adenine methylase (“Dam”).

89. The immunogenic composition of claim 88 wherein the protein is cyclic AMP receptor protein (“Crp”).

90. The immunogenic composition of claim 89 wherein the pathogen is selected from the group consisting of Streptococcus pneumoniae, Streptococcus pyogenes, Streptococcus group B, Streptococcus mutans, Streptococcus sobrinus, Streptococcus equi, Staphylococcus spp., Erysipelothrix rhusiopathiae, Bacillus anthracis, Listeria monocytogenes, Mycobacterium leprae, Mycobacterium tuberculosis, Clostridium tetani, Clostridium botulinum, Clostridium difficile, Clostridium perfringens, Corynebacterium diphtheriae, and Mycoplasma spp.

91. The immunogenic composition of claim 90 wherein the pathogen is Streptococcus pneumoniae.

92. The immunogenic composition of claim 80 wherein the third polynucleotide encodes a sequence as set forth in SEQ ID NO:2 or SEQ ID NO:3.

93. The immunogenic composition of claim 91 wherein the first antigen is a pneumococcal surface protein A (“PspA”).

94. The immunogenic composition of claim 93 wherein the third polynucleotide encodes a sequence as set forth in SEQ ID NO:2 or SEQ ID NO:3.

95. The immunogenic composition of claim 94 wherein the first polynucleotide encodes a sequence as set forth in SEQ ID NO:1, the second polynucleotide encodes a pneumococcal surface protein A from Streptococcus pneumoniae strain Rx1, and the third polynucleotide encodes a sequence as set forth in SEQ ID NO:2.

96. The immunogenic composition of claim 95 wherein the first polynucleotide, the second polynucleotide, and the third polynucleotide together comprise the sequence as set forth in SEQ ID NO:20.

97. The immunogenic composition of claim 96 wherein the strain of bacteria is a Salmonella enterica, which further comprises (a) a mutation that renders the gene encoding cyclic AMP receptor protein inactive, (b) an inactivating mutation in the chromosomal gene encoding aspartate β-semialdehyde dehydrogenase (Asd) and (c) a plasmid, which comprises (i) the first polynucleotide, (ii) the second polynucleotides, (iii) the third polynucleotides and (iv) a polynucleotide that encodes a functional Asd.
